(19) United States
(12) Patent Application Publication
(10) Pub. No.: US 2026/0121033 A1
(43) Pub. Date: Apr. 30, 2026

(54) NIOBIUM-BASED NEGATIVE ELECTRODES AND HIGH ENERGY DENSITY RECHARGEABLE LITHIUM-ION BATTERIES WITH THE SAME

(71) Applicants: Medtronic, Inc., Minneapolis, MN (US); The Royal Institution for the Advancement of Learning/McGill University, Montreal (CA)

(72) Inventors: Hui Ye, Maple Grove, MN (US); Prabhakar A. Tamirisa, Brooklyn Park, MN (US); Eric McCalla, Brossard, Quebec (CA); James M. Sieffert, Montreal, Quebec (CA); Christopher J. Lang, Montreal, Quebec (CA)

(21) Appl. No.: 18/895,970

(22) Filed: Sep. 25, 2024

Related U.S. Application Data
(60) Provisional application No. 63/541,456, filed on Sep. 29, 2023.
(60) Provisional application No. 63/564,723, filed on Mar. 13, 2024.
(60) Provisional application No. 63/675,905, filed on Jul. 26, 2024.

(51) Int. Cl. H01M 4/485; H01M 4/02; H01M 4/36; H01M 4/587; H01M 4/66; H01M 10/0525

(52) U.S. Cl. CPC H01M 4/485; H01M 4/366; H01M 4/587; H01M 4/661; H01M 10/0525; H01M 2004/027; H01M 2220/30

(57) ABSTRACT

Rechargeable lithium-ion cells and implantable medical devices including the same are provided herein with niobium-based negative electrodes and high voltage positive electrodes. Electrode active materials are provided herein, including pseudoternary compositions within a Ti—Nb—W—O pseudoternary system and/or including titanium, niobium, oxygen, and an elemental dopant. Rechargeable lithium-ion cells provided herein with niobium-based negative electrodes afford improved energy density, and, more particularly, provide improved energy density at operating voltages for use in implantable medical devices.

### RELATED APPLICATIONS

This application claims the benefit of U.S. Provisional Application No. 63/541,456, filed on Sep. 29, 2023, titled TITANIUM-NIOBIUM-TUNGSTEN-OXIDE NEGATIVE ELECTRODE COMPOSITES; U.S. Provisional Application No. 63/564,723, filed on Mar. 13, 2024, titled DOPED TITANIUM NIOBIUM OXIDE ANODE MATERIAL; and U.S. Provisional Application No. 63/675,905, filed on Jul. 26, 2024, titled HIGH ENERGY DENSITY RECHARGEABLE LITHIUM ION BATTERY WITH NIOBIUM-BASED NEGATIVE ELECTRODE, each of which is hereby incorporated by reference in its entirety.

### SUMMARY

The present disclosure generally relates to the field of lithium-ion batteries or cells, and more specifically, lithium-ion cells with niobium-based negative electrodes, negative electrode compositions, and methods of making negative electrode compositions for use in lithium-ion batteries or cells.

Lithium-ion batteries or cells (i.e., electrochemical cells) include one or more positive electrodes, one or more negative electrodes, and an electrolyte material provided within a case or housing. Separators made from a porous polymer or other suitable material may also be provided between the positive and negative electrodes to prevent direct contact between adjacent electrodes, and electrolyte material penetrates through pores in this porous polymer.

Rechargeable lithium-ion batteries have become the primary power source for portable electronics. With increasing global demand for renewable and efficient power sources, many negative electrode materials have been investigated for lithium-ion batteries. For example, materials with high energy densities are generally desirable, such as for consumer electronic device, medical device, or electric vehicle applications. Long-term service life is an important characteristic for rechargeable lithium-ion batteries, especially in applications such as implantable medical devices, which often require 10 or more years of service life. Energy density and power density are two key attributes of a battery. Energy density is the amount of energy in the battery compared to the volume of the battery and may be expressed, for example, as milliwatt-hours per cubic centimeter (mWh/cm³). Discharge capacity or specific capacity is the amount of energy in the battery compared to the mass of the battery and may be expressed, for example, as milliamp-hours per gram (mAh/g). Batteries with greater specific capacity tend to last longer in discharge and may allow for miniaturization, such as for use in small form factor devices. Power density is the time rate of energy transfer of which a battery is capable and may be expressed, for example, in watts per liter (W/L). Both higher energy density and higher power density are generally desirable. For smaller batteries-like those used in implantable medical devices-high energy density, high power density, reliability, and long service life are all important attributes.

In general, there is a need for electrochemical cells with improved energy density, capacity retention, rate retention, and long-term stability. Likewise, in general, there is a need for a negative electrode active material to afford electrochemical cells with improved electrochemical performance characteristics (e.g., specific capacity, capacity retention, and/or rate retention) that are also capable of high-current applications. Such cells could improve the lives, comfort, and quality of care for patients living with or receiving implantable medical devices such as pacemakers, insulin pumps, cardioverter-defibrillators, drug delivery pumps, and neurostimulators.

Embodiments disclosed herein may include a rechargeable lithium-ion cell with a negative electrode, a positive electrode, a separator between the negative electrode and the positive electrode, an electrolyte material, and an operating voltage of 1.5 V to 4 V or 1.8 V to 3.4 V. The negative electrode includes a negative electrode current collector and a negative electrode active material with a niobium-based oxide. The positive electrode includes a positive electrode current collector and a positive electrode active material with a lithium transition metal oxide. Embodiments herein may also include an implantable medical device with the rechargeable lithium-ion cell described herein.

The niobium-based oxide may be, or include, Nb₂O₅, a Nb—Ti—O oxide, a Nb—W—O oxide, a Nb—Ti—W—O oxide, or a combination of two or more thereof. The niobium-based oxide may be represented by the formula Nb_{1-x-y}TiₓW_{y}O_{z}. The niobium-based oxide may be, or include, a pseudoternary composition with a metal portion having a niobium fraction, a titanium fraction, and a tungsten fraction. The pseudoternary composition may be represented by the formula: Nb_{1-x-y} TiₓW_{y}O_{z}. The niobium-based oxide may be, or include, a metal portion with a titanium fraction, a niobium fraction, a doped fraction having a dopant, and an oxygen portion. The metal portion may include 10 atom-% or less, 5 atom-% or less, or between 0.1 atom-% and 5 atom-% of the doped metal fraction, and an oxygen portion. The niobium-based oxide may be, or include, a metal portion with a titanium fraction, a niobium fraction, a doped metal fraction, and an oxygen portion. The doped metal fraction may include rhenium, cerium, praseodymium, neodymium, promethium, samarium, europium, gadolinium, dysprosium, holmium, erbium, thulium, ytterbium, or a combination of two or more thereof.

The negative electrode may further include a carbon coating, a binder, an electrically conductive additive, or a combination of two or more thereof. The negative electrode active material may include the carbon coating. The negative electrode active material may have a lithiation potential compared to Li+/Li of between 0.5 V and 3.5 V or between 1.0 V and 3.0 V. The negative electrode may have a negative electrode area specific capacity of between 1 mAh/cm² and 6 mAh/cm².

The lithium transition metal oxide may be, or include, LiCoO₂, LiCoMnO₄, LiCoPO₄, LiNi_{0.5}Mn_{1.5}O₄, a composition represented by the formula LiNiₓMn_{y}Co_{1-x-y}O₂, a composition represented by the formula LiNiₓCo_{y}Al_{1-x-y}O₂, a lithium-rich layered oxide represented by the formula Li₁+xTM₁−xO₂ (wherein TM represents a transition metal), or a combination of two or more thereof. The positive electrode active material may have an operating electrode potential compared to Li+/Li of between 2.8 V and 5 V or between 3 V and 4.5 V. The positive electrode may have a positive electrode area specific capacity of between 1 mAh/cm² and 6 mAh/cm².

The negative electrode may define a negative electrode major surface and one or more negative electrode edges defining a perimeter of the negative electrode major surface, the positive electrode may define a positive electrode major surface and one or more positive electrode edges defining a perimeter of the positive electrode major surface, and the one or more positive electrode edges may be arranged line-to-line with the one or more negative electrode edges.

The positive electrode may have a positive electrode specific capacity and a positive electrode capacity fade rate, and the negative electrode may have a negative electrode specific capacity and a negative electrode capacity fade rate. A N/P capacity ratio of the negative electrode specific capacity to the positive electrode specific capacity may be less than 1 when the negative electrode capacity fade rate is less than the positive electrode capacity fade rate, and greater than 1 when the negative electrode capacity fade rate is greater than the positive electrode capacity fade rate.

The positive electrode may have a positive electrode specific capacity and a positive electrode capacity fade rate, and the negative electrode may have a negative electrode specific capacity and a negative electrode capacity fade rate. A N/P capacity ratio of the negative electrode specific capacity to the positive electrode specific capacity may be less than 1, and the negative electrode capacity fade rate may be less than the positive electrode capacity fade rate.

The positive electrode may have a positive electrode specific capacity and a positive electrode capacity fade rate, and the negative electrode may have a negative electrode specific capacity and a negative electrode capacity fade rate. A N/P capacity ratio of the negative electrode specific capacity to the positive electrode specific capacity may be greater than 1, and the negative electrode capacity fade rate may be greater than the positive electrode capacity fade rate.

The positive electrode may have a positive electrode first-cycle irreversible capacity, and the negative electrode may have a negative electrode first-cycle irreversible capacity. The negative electrode current collector may be, or include, copper when the negative electrode first-cycle irreversible capacity is less than the positive electrode first-cycle irreversible capacity, and aluminum when the negative electrode first-cycle irreversible capacity is greater than the positive electrode first-cycle irreversible capacity.

The positive electrode may have a positive electrode first-cycle irreversible capacity, and the negative electrode may have a negative electrode first-cycle irreversible capacity. The negative electrode active material may have an operating voltage of 3 V or greater at 95% delithiation, when the negative electrode first-cycle irreversible capacity is greater than the positive electrode first-cycle irreversible capacity. The positive electrode active material may have an operating voltage of 3 V or less at 95% lithiation, when the negative electrode first-cycle irreversible capacity is less than the positive electrode first-cycle irreversible capacity.

The negative electrode current collector may be, or include, aluminum when the negative electrode first-cycle irreversible capacity is greater than the positive electrode first-cycle irreversible capacity. The negative electrode current collector may be, or include, copper when the negative electrode first-cycle irreversible capacity is less than the positive electrode first-cycle irreversible capacity.

The positive electrode may have a positive electrode first-cycle irreversible capacity, and the negative electrode may have a negative electrode first-cycle irreversible capacity. The negative electrode active material may have an operating voltage of 3 V or greater at 95% delithiation and the negative electrode first-cycle irreversible capacity may be greater than the positive electrode first-cycle irreversible capacity.

The positive electrode may have a positive electrode first-cycle irreversible capacity, and the negative electrode may have a negative electrode first-cycle irreversible capacity. The positive electrode active material may have an operating voltage of 3 V or less at 95% lithiation, and the negative electrode first-cycle irreversible capacity may be less than the positive electrode first-cycle irreversible capacity.

The positive electrode may have a positive electrode first-cycle irreversible capacity, and the negative electrode may have a negative electrode first-cycle irreversible capacity. The negative electrode active material may have an operating voltage of 3 V or greater at the end of near full delithiation, the negative electrode first-cycle irreversible capacity may be greater than the positive electrode first-cycle irreversible capacity, and the negative electrode current collector may be, or include, aluminum.

The positive electrode may have a positive electrode first-cycle irreversible capacity, and the negative electrode may have a negative electrode first-cycle irreversible capacity. The negative electrode active material may have an operating voltage of 3 V or less at the end of near full delithiation, the negative electrode first-cycle irreversible capacity may be less than the positive electrode first-cycle irreversible capacity, and the negative electrode current collector may be, or include, copper.

In further respects, negative electrode active material compositions are described herein with numerous single-phase materials made within the ternary of niobium (Nb), titanium (Ti), and tungsten (W). These single-phase compositions may generally demonstrate improved performance, such as improved room temperature discharge capacities, improved capacity retentions in room temperature cycling, improved discharge capacities at 37° C., and/or improved capacity retentions in 37° C. cycling.

For high power applications such as in medical devices, and more specifically implantable medical devices, Li₄Ti₅O₁₂ (LTO) may commonly be used as a negative electrode active material. LTO has a relatively high capacity retention but may have limited specific capacities with a theoretical capacity of about 175 milliamp hours per gram (mAh/g). While graphite operates near the potentials of lithium plates, LTO operates at approximately 1.5 volts (V) compared with lithium plates, affording improved electrolyte stability, which generally improves extended cycling and reduces lithium-dendrite formation. As such, there is generally a need for negative electrode materials with improved discharge capacities that operate near 1.5 V, which may be useful in batteries for devices in which both high power application and high discharge capacity are desirable, such as medical devices, and implantable medical devices, more specifically. For example, each of TiNb₂O₇, Nb₁₆W₅O₅₅, and Nb₁₈W₁₆O₉₃ relies on the electrochemical activity of Nb, and each affords improved discharge capacities over LTO, but generally suffer in other parameters, such as relatively low capacity retention. For example, Nb₂TiO₇ has a theoretical capacity of 387 mAh/g if all Nb could be reduced to 3+, but may typically only achieve 285 mAh/g. As another example, Nb₁₆W₅O₅₅ may typically achieve capacities near 225 mAh/g.

As described herein, negative electrode active materials including pseudoternary compositions within a Ti—Nb—W—O pseudoternary system as well as electrodes and electrochemical cells including the same and methods of making the same may be provided.

Embodiments disclosed herein may include a negative electrode composite including a negative electrode active material represented by the formula Nb_{1-x-y}TiₓW_{y}O_{z}. The negative electrode composite may include a binder and an electrically conductive additive. The binder may include polyvinylidene fluoride. The electrically conductive additive may include one or both of carbon black and carbon nanotubes. The negative electrode composite may have a porosity of 50% or less, 15% or greater, or between 15% and 50%. The electrode composite may have a surface area of 300 cm² or less, 0.5 cm² or greater, or between 0.5 cm² and 300 cm². The negative electrode active material may define a crystal morphology including one or more of octahedra centered by Nb and Ti, octahedra centered by Nb and W, octahedra centered by Nb and Ti and W, octahedra centered by W, or tetrahedra centered by W. The negative electrode active material may define a crystal morphology with an average crystal size 2 micrometers (um) or less, 1 um or less, 0.5 um or less, between 0.1 um and 5 um, or between 0.3 um and 1 um.

Embodiments disclosed herein may further include a negative electrode including the negative electrode composite provided herein and a negative electrode current collector electrically coupled to the negative electrode composite. The negative electrode may further include a coating layer on a surface of the negative electrode. The coating layer may include carbon. A surface area of the negative electrode may be 300 cm² or less, 0.5 cm² or greater, or between 0.5 cm² and 300 cm².

Embodiments disclosed herein may still further include an electrode composite, a binder, and an electrically conductive additive. The electrode composite may include a pseudoternary composition with a metal fraction including a niobium fraction, a titanium fraction, and a tungsten fraction. The pseudoternary composition may be represented by the formula Nb_{1-x-y} TiₓW_{y}O_{z}. The binder may include polyvinylidene fluoride. The electrically conductive additive may include one or both of carbon black and carbon nanotubes. The electrode composite may have a porosity of 50% or less, 15% or greater, or between 15% and 50%. The electrode composite may have a surface area of 300 cm² or less, 0.5 cm² or greater, or between 0.5 cm² and 300 cm². The pseudoternary composition may define a crystal morphology including one or more of octahedra centered by Nb and Ti, octahedra centered by Nb and W, octahedra centered by Nb and Ti and W, octahedra centered by W, or tetrahedra centered by W. The pseudoternary composition may define a crystal morphology with an average crystal size of 2 um or less, 1 um or less, 0.5 um or less, between 0.1 um and 5 um, or between 0.3 um and 1 um. The niobium fraction may be 40 atomic percent (at. %) or greater, 45 at. % or greater, 50 at. % or greater, 55 at. % or greater, 60 at. % or less, 55 at. % or less, 45 at. % or less, or between 40 at. % and 60 at. %. The titanium fraction may be 50 at. % or less, 45 at. % or less, 40 at. % or less, 35 at. % or less, 30 at. % or greater, 35 at. % or greater, 40 at. % or greater, 45 at. % or greater, or between 30 at. % and 50 at. %. The tungsten fraction may be 25 at. % or less, 20 at. % or less, 15 at. % or less, 10 at. % or less, 5 at. % or greater, 10 at. % or greater, 15 at. % or greater, 20 at. % or greater, between 5 at. % and 25 at. %, or between 30 at. % and 75 at. %.

Embodiments disclosed herein may yet further include an electrode including the electrode composite provided herein and a current collector electrically coupled to the electrode composite. The electrode may further include a coating layer on a surface of the electrode. The coating layer may include carbon. The electrode may have a surface area of 300 cm² or less, 0.5 cm² or greater, or between 0.5 cm² and 300 cm².

Embodiments disclosed herein may still yet further include an electrochemical cell including a negative electrode, a positive electrode, a separator between the negative electrode and the positive electrode, and an electrolyte. The negative electrode may have a negative electrode active material including a pseudoternary composition with a metal fraction including a niobium fraction, a titanium fraction, and a tungsten fraction. The electrochemical cell may have a specific capacity at ambient room temperature of 200 mAh/g or greater, 250 mAh/g or greater, 300 mAh/g or greater, or 350 mAh/g or greater. The electrochemical cell may have a specific capacity at an ambient temperature of 37° C. of 200 mAh/g or greater, 250 mAh/g or greater, 300 mAh/g or greater, or 350 mAh/g or greater. The electrochemical cell may have an average discharge voltage at ambient room temperature of 2 V or less, 1.8 V or less, 1.7 V or less, 1.6 V or less, or 1.5 V or less. The electrochemical cell may have an average discharge voltage at an ambient temperature of 37° C. of 2 V or less, 1.8 V or less, 1.7 V or less, 1.6 V or less, or 1.5 V or less. The electrochemical cell may have an energy capacity retention after 10 charge-discharge cycles at ambient room temperature of 80% or greater, 90% or greater, 95% or greater, 97% or greater, 98% or greater, or 99% or greater. The electrochemical cell may have an energy capacity retention after 10 charge-discharge cycles at an ambient temperature of 37° C. of 80% or greater, 90% or greater, 95% or greater, 97% or greater, 98% or greater, or 99% or greater. The electrochemical cell may have a discharge rate retention after 10 charge-discharge cycles at room temperature of 75% or greater, 80% or greater, 85% or greater, 90% or greater, or 95% or greater. The electrochemical cell may have a discharge rate retention after 10 charge-discharge cycles at an ambient temperature of 37° C. of 75% or greater, 80% or greater, 85% or greater, 90% or greater, or 95% or greater. The electrochemical cell may have a diffusivity at ambient room temperature of 5×10⁻⁷ cm²/s or less, 1×10⁻⁷ cm²/s or less, or 5×10⁸ cm²/s or less. The electrochemical cell may have a diffusivity at an ambient temperature of 37° C. of 5×10⁻⁷ cm²/s or less, 1×10⁻⁷ cm²/s or less, or 5×10⁸ cm²/s or less.

Embodiments disclosed herein may further include an electrochemical cell having a negative electrode, a positive electrode, a separator between the negative electrode and the positive electrode, and an electrolyte. The negative electrode may include a negative electrode active material represented by the formula Nb_{1-x-y}TiₓW_{y}O_{z}. The electrochemical cell may have a specific capacity at ambient room temperature of 200 mAh/g or greater, 250 mAh/g or greater, 300 mAh/g or greater, or 350 mAh/g or greater. The electrochemical cell may have a specific capacity at an ambient temperature of 37° C. of 200 mAh/g or greater, 250 mAh/g or greater, 300 mAh/g or greater, or 350 mAh/g or greater. The electrochemical cell may have an average discharge voltage at ambient room temperature of 2 V or less, 1.8 V or less, 1.7 V or less, 1.6 V or less, or 1.5 V or less. The electrochemical cell may have an average discharge voltage at an ambient temperature of 37° C. of 2 V or less, 1.8 V or less, 1.7 V or less, 1.6 V or less, or 1.5 V or less. The electrochemical cell may have energy capacity retention after 10 charge-discharge cycles at ambient room temperature of 80% or greater, 90% or greater, 95% or greater, 97% or greater, 98% or greater, or 99% or greater. The electrochemical cell may have an energy capacity retention after 10 charge-discharge cycles at an ambient temperature of 37° C. of 80% or greater, 90% or greater, 95% or greater, 97% or greater, 98% or greater, or 99% or greater. The electrochemical cell may have a discharge rate retention after 10 charge-discharge cycles at room temperature of 75% or greater, 80% or greater, 85% or greater, 90% or greater, or 95% or greater. The electrochemical cell may have a discharge rate retention after 10 charge-discharge cycles at an ambient temperature of 37° C. of 75% or greater, 80% or greater, 85% or greater, 90% or greater, or 95% or greater. The electrochemical cell may have a diffusivity at ambient room temperature of 5×10⁻⁷ cm²/s or less, 1×10⁻⁷ cm²/s or less, or 5×10⁸ cm²/s or less. The electrochemical cell may have a diffusivity at an ambient temperature of 37° C. of 5×10⁻⁷ cm²/s or less, 1×10⁻⁷ cm²/s or less, or 5×10⁻⁸ cm²/s or less.

Embodiments disclosed herein may still further include a method of forming a negative electrode active material, the method including preparing a sol-gel suspension, gelating the sol-gel suspension to form a wet gel, off-gassing the wet gel to form a dry gel, and calcinating the dry gel to form the negative electrode active material. The sol-gel suspension may include a tungsten compound, a titanium compound, a niobium compound, and a chelating agent. The chelating agent may include one or more of citric acid, EDTA, or glycol. The gelating the sol-gel suspension to form the wet gel may occur in a vacuum or a partial vacuum. The gelating the sol-gel suspension to form the wet gel may occur at an ambient temperature between 50° C. and 250° C., 60° C. or greater, 110° C. or greater, 200° C. or greater, 250° C. or less, 120° C. or less, or 80° C. or less. The gelating the sol-gel suspension to form the wet gel may occur at an ambient temperature of 60° C. or greater, 110° C. or greater, or 200° C. or greater. The gelating the sol-gel suspension to form the wet gel may include heating the sol-gel suspension at an ambient temperature ramping from 50° C. or greater to 200° C. or greater. The off-gassing the wet gel to form the dry gel may occur at an ambient temperature of 300° C. or greater, 400° C. or greater, 450° C. or less, 350° C. or less, or between 300° C. and 600° C. The calcinating the dry gel to form the negative electrode active material may occur at an ambient temperature of 800° C. or greater, 1,000° C. or greater, 1,200° C. or less, 900° C. or less, or between 800° C. and 1,500° C. The tungsten compound may be one or both of tungsten trioxide and ammonium metatungstate hydrate. The titanium compound may be one or both of titanium butoxide and titanium(IV) bis(ammonium lactato)dihydroxide. The niobium compound may be ammonium niobate oxalate hydrate.

In still further respects, the present disclosure describes a negative electrode active material. The negative electrode active material may be included in a negative electrode composite. The negative electrode composite may be included in a negative electrode for use in a battery. The negative electrode active material of the present disclosure may have a high specific capacity at a physiologically relevant working temperature. Negative electrode active material having a high specific capacity at a physiologically relevant working temperature may allow for the miniaturization of batteries containing the same for applications in medical devices, such as implantable medical devices.

The negative electrode active material of the present disclosure includes a metal portion and an oxygen portion. The metal portion includes a titanium (Ti) fraction, a niobium (Nb) fraction, and a doped fraction. In some embodiments, the doped fraction includes a dopant, and the metal portion includes 10 wt-% or less or 5 wt-% or less of the doped fraction. In some embodiments, the doped metal fraction includes rhenium (Re), cerium (Ce), praseodymium (Pr), neodymium (Nd), promethium (Pm), samarium (Sm), europium (Eu), gadolinium (Gd) dysprosium (Dy), holmium (Ho), erbium (Er), Thulium (Tm), Ytterbium (Yt); or any combination thereof.

The negative electrode active material of the present disclosure may be of the formula (TiNb₂)₍₁₋ₓ₎M₍₃ₓ₎O₇ wherein M is a dopant and x is greater than 0 and less than 1. In some embodiments, M includes transition metal, an alkali metal, an alkaline metal, a lanthanide, a post transition metal, a metalloid, or any combination thereof. In some embodiments, x is 0.01 to 0.30, 0.01 to 0.3, 0.01 to 0.25, 0.01 to 0.15, 0.01 to 0.1, 0.01 to 0.08, 0.02 to 0.08, 0.02 to 0.07, 0.02 to 0.06, 0.05 to 0.1, or 0.05 to 0.08.

In some embodiments, the negative electrode active material is included in a negative electrode composite. The negative electrode composite may further include a binder and an electrically conductive active.

In some embodiments, the negative electrode active material has a specific capacity at 20° C. to 25° C. of 300 mAh/g or greater, 310 mAh/g or greater, 320 mAh/g or greater, 340 mAh/g or greater, or 350 mAh/g or greater as measured according to the Specific Capacity Test Method.

The negative electrode active material or negative electrode composite containing the same may be included in an electrode such as a negative electrode. The electrode includes the negative electrode active material or the negative electrode composite and a current collector. The current collector is electrically coupled to the electrode.

In some embodiments, the negative electrode has a has an average discharge voltage at 20° C. to 25° C. of 2.2 volts (V) or less, 1.8 V or less, 1.7 V or less, 1.6 V or less, 1.5 V, 1.4 V or less, or 1.2 V or less vs Li/Li+ as measured according to the Average Discharge Test Method.

An electrode that includes the negative electrode active material or negative electrode composite containing the same may be included in an electrochemical cell. The electrochemical cell further includes a positive electrode, a separator between the negative electrode and the positive electrode, and an electrolyte.

Embodiments disclosed herein may still further include an implantable medical device including the rechargeable lithium-ion cell of one or more embodiments described herein.

### BRIEF DESCRIPTION OF DRAWINGS

FIG. 1 is a diagrammatic representation of a portion of an illustrative lithium-ion battery.

FIG. 2A is a diagrammatic cross-sectional view of a portion of an illustrative battery or cell.

FIG. 2B is a diagrammatic cross-sectional view of a portion of an illustrative battery or cell having negative electrode active material provided on both sides of the negative electrode current collector.

FIG. 3A shows a perspective view of an illustrative electrode stack that includes electrodes arranged line-to-line.

FIG. 3B shows an exploded perspective view of the illustrative electrode stack of FIG. 3A.

FIG. 4 is a schematic view of a system in the form of an implantable medical device implanted within a body or torso of a patient.

FIG. 5 is a block diagram illustrating an embodiment of a sol-gel method of synthesizing a negative electrode active material including a pseudoternary composition.

FIGS. 6A-6C are schematic drawings of examples of suitable crystal structures of embodiments of pseutoternary compositions.

FIG. 7 is a graphical representation of a phase stability diagram of embodiments of Nb_{1-x-y}TiₓW_{y}O_{z} pseudoternary systems based on x-ray diffraction (PXRD) analysis shown on a Gibbs' triangle displaying solid-solution regions.

FIGS. 8A-8B are graphical representations of PXRD patterns along two pseudoternary composition lines. FIG. 8A shows PXRD patterns on compositions lines between samples B2 and C2 of FIG. 7. FIG. 8B shows PXRD patterns on composition lines between samples A3 and B3 of FIG. 7.

FIGS. 9A-9C are scanning electron microscope (SEM) images of three illustrative pseudoternary compounds of FIG. 7 within single-phase regions within the ternary space and three corresponding pseudobinary compounds of FIG. 7.

FIGS. 10A-10L are graphical representations of cyclic voltammetry (CV) plots showing electrochemical performance of selected pseudoternary and pseudobinary samples of FIG. 7. Graphical representations of CVs are shown for sample A1 at ambient room temperature (10A) and at 37° C. ambient temperature (10B), sample A2 at ambient room temperature (10C) and at 37° C. ambient temperature (10D), sample B1 at ambient room temperature (10E) and at 37° C. ambient temperature (10F), sample B4 at ambient room temperature (10G) and at 37° C. ambient temperature (10H), sample C1 at ambient room temperature (10I) and at 37° C. ambient temperature (10J), and sample C2 at ambient room temperature (10K) and at 37° C. ambient temperature (10L).

FIGS. 11A-11G are graphical representations of voltage curves for selected samples of FIG. 7 with Li metal used as the counter electrode. Graphical representations of voltage curves are shown for sample A1 at ambient room temperature (11A), sample B1 at ambient room temperature (11B), sample A2 at ambient room temperature (11C) and at 37° C. ambient temperature (11D), sample B4 at ambient room temperature (11E) and at 37° C. ambient temperature (11F), and sample C2 at ambient room temperature (11G).

FIGS. 12A-12F are graphical representations of electrochemical performance metrics of the samples of FIG. 7 plotted over the Gibbs' triangle of FIG. 7. Graphical representations of first discharge capacities are shown at ambient room temperature (12A) and at 37° C. ambient temperature (12B), of energy capacity retention after 10 cycles at ambient room temperature (12C) and at 37° C. ambient temperature (12D), and of discharge rate retention at ambient room temperature (12E) and at 37° C. ambient temperature (12F).

FIG. 13 is an array of images of TNO and TNO doped materials. The dopants used are presented in the table, with “Und” indicating undoped.

FIG. 14 shows SEM images of TNO and TNO doped materials. The scale is 500 nm (scale bar) and is the same for each image.

FIGS. 15A and 15B are powder X-ray diffraction traces on composition lines for Nd-TNO (15A) and Tb-TNO (15B).

FIG. 16 is a pseudoternary diagram of Nb—Ti-M-O (M is an elemental dopant), with a black region denoting the TNO phase stability region investigated for TNO samples doped with either Nd or Tb.

FIGS. 17A-17C are plots showing the relative phase composition (calculated as the height of the largest secondary phase XRD peak divided by the height of the largest TNO peak; 17A), the unit cell volume (17B), and the specific capacity (17C) for Nd-TNO and Tb-TNO materials of various compositions. In all pseudoternaries, the lowest dopant content is 0.4 atom-%, the highest is 3.2 atom-%, and the others are evenly spaced between. The star indicates the 2 atom-% doped composition.

FIGS. 18A-18C are heat maps showing discharge capacity (18A), discharge capacity of the tenth cycle (18B), and discharge capacity at 2 V per hour (18C) of cells having various elemental dopants in a TNO negative electrode active material.

FIGS. 19A and 19B are plots showing a vs b (19A) and a vs c (19B) lattice parameters of undoped TNO (circles) and TNO doped with various elements (squares).

FIG. 20 shows cyclic voltammograms of the first cycle at 0.1 V h⁻¹ between 1 V and 3 V vs. Li at room temperature of various doped TNO materials.

FIGS. 21A-21C are plots showing specific first discharge capacity cycled at 0.1 V h⁻¹ between 1 V and 3 V vs. Li (21A), capacity retention after ten cycles (21B), and rate performance for each sample (the discharge capacity obtained at 2 V h⁻¹ divided by the discharge capacity of the first cycle at 0.1 V h⁻¹) (21C) of undoped TNO (dashed line) and various doped TNO samples at room temperature and 37° C.

FIG. 22 shows plots of irreversible capacity and calculated diffusion coefficients (D) of various doped TNO samples at room temperature (squares) and 37° C. (circles).

FIG. 23 shows plots comparing discharge capacity and capacity retention of various doped TNO samples at room temperature (squares) and 37° C. (circles) verse the ionic radius of the dopant element. The shaded region shows the region of ionic radius between Nb and Tb.

FIGS. 24A and 24B are plots showing relative phase composition (calculated as the height of the largest secondary phase XRD peak divided by the height of the largest TNO peak; 24A) and specific capacity (24B) for Cs-TNO and Dy-TNO materials of various compositions.

FIG. 25 shows voltage curves for various doped TNO materials derived from the cyclic voltammograms of FIG. 20.

FIG. 26 is a plot showing average voltage of various doped TNO materials.

FIG. 27 is a plot showing the relationship between the Shannon radius of various elemental dopants in doped TNO samples and the logarithm diffusion coefficient (D) of the doped TNO material at room temperature.

The figures are rendered primarily for clarity and, as a result, are not necessarily drawn to scale. Moreover, various structure/components may be shown diagrammatically or removed from some of or all the views to better illustrate aspects of the depicted embodiments, or where inclusion of such structure/components is not necessary to an understanding of the various illustrative embodiments described herein. The lack of illustration/description of such structures/components in a particular figure is, however, not to be interpreted as limiting the scope of the various embodiments in any way.

### DETAILED DESCRIPTION

All scientific and technical terms used herein have meanings commonly used in the art, unless otherwise specified. The definitions provided herein are to facilitate understanding of certain terms used frequently herein and are not meant to limit the scope of the present disclosure.

Unless otherwise indicated, the terms “polymer,” “polymerized monomers,” and “polymeric material” include, but are not limited to, organic homopolymers, copolymers, such as, for example, block, graft, random and alternating copolymers, terpolymers, etc., and blends and modifications thereof. Furthermore, unless otherwise specifically limited, the term “polymer” shall include all possible geometrical configurations of components, such as repeating units, of the polymer material. These configurations include, but are not limited to, isotactic, syndiotactic, and atactic symmetries.

In this disclosure, all numbers are assumed to be modified by the term “about,” which encompasses the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The recitations of numerical ranges by endpoints include all numbers subsumed within that range (e.g., 1 to 5 includes 1, 1.5, 2, 2.75, 3, 3.80, 4, 5, etc. and 10 or less includes 10, 9.4, 7.6, 5, 4.3, 2.9, 1.62, 0.3, etc.). Where a range of values is “up to,” “at most,” or “at least” a particular value, that value is included within the range.

As used herein, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Such inclusive or open-ended words encompass more restrictive terms or phrases, such as “consisting essentially,” or closed terms or phrases, such as “consisting”.

Reference in the specification to “some embodiments,” “an embodiment,” “one embodiment,” “one or more embodiments,” “embodiments,” “at least one embodiment”, or “other embodiments” means that a particular feature, structure, or characteristic described in connection with the embodiments is included in at least one or more embodiments, but not necessarily all embodiments, of the present disclosure. To the extent that the disclosure describes aspects, components, or elements associated with a particular embodiment in more detail or breadth, it is contemplated that the aspects, components, or elements associated with such embodiment should be understood to encompass the additional detail and breadth described in the disclosure.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the present disclosure.

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components.

As described herein, lithium-ion batteries having negative electrode active materials including niobium-based oxides provide improved characteristics, including improved energy density. More particularly, lithium-ion batteries described herein having niobium-oxide based negative electrodes and high voltage positive electrodes provide improved characteristics, including improved energy density at desirable operating voltages for use in implantable medical devices.

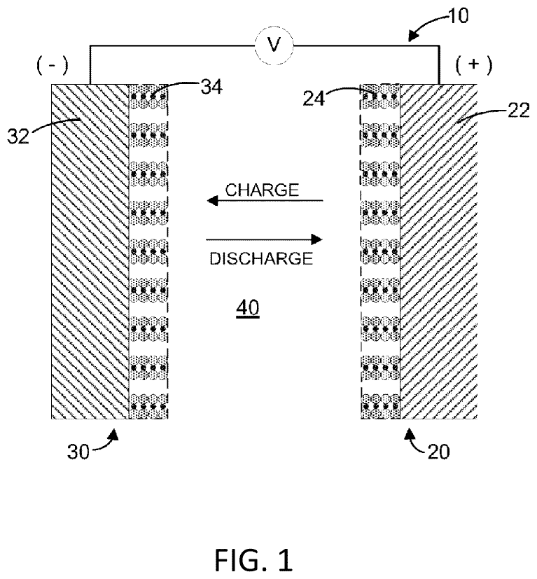

A diagrammatic representation of a portion of an illustrative rechargeable lithium-ion cell 10 is shown in FIG. 1. The cell 10 includes a positive electrode 20 that includes a positive electrode current collector 22 and a positive electrode active material 24, a negative electrode 30 that includes a negative electrode current collector 32 and a negative electrode active material 34, an electrolyte material 40, and a separator (e.g., a polymeric microporous separator, not shown) provided intermediate, or between, the positive electrode 20 and the negative electrode 30. The electrodes 20, 30 may be provided as relatively flat or planar plates or may be wrapped or wound in a spiral or other configuration (e.g., an oval configuration). It will be understood in light of the present disclosure that any suitable electrochemical cell geometry or construction (such as rectangular, prismatic, cylindrical, oval, coiled, stacked, folded, etc.) may be used and the disclosure is not limited in this regard. The electrodes 20, 30 may also be provided in a folded configuration. The battery 10 may be at least partially disposed within (e.g., enclosed within) a housing (not shown).

During charging and discharging of the battery 10, lithium ions move between the positive electrode 20 and the negative electrode 30. For example, when the battery 10 is being discharged, lithium ions flow from the negative electrode 30 to the positive electrode 20. In contrast, when the battery 10 is being charged, lithium ions flow from the positive electrode 20 to the negative electrode 30.

Once assembly of the battery is complete, an initial charging operation (referred to as a “formation process”) may be performed. During the formation process, a stable solid-electrolyte inter-phase (SEI) layer is formed at the negative electrode and, in some cases, at the positive electrode. These SEI layers may act to passivate the electrode-electrolyte interfaces and may prevent side-reactions thereafter.

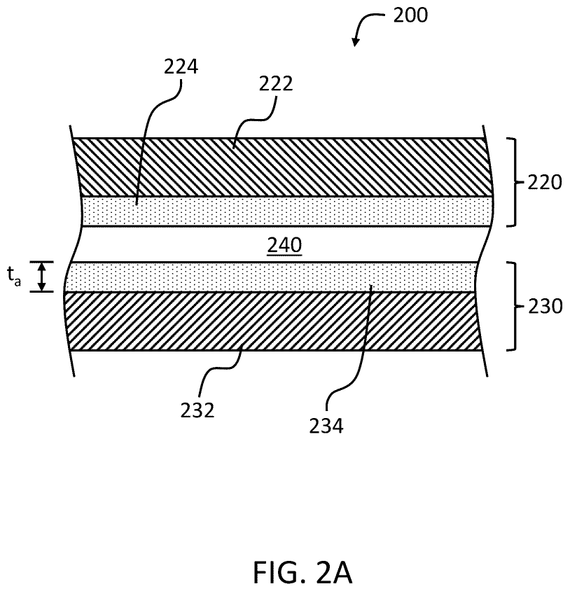

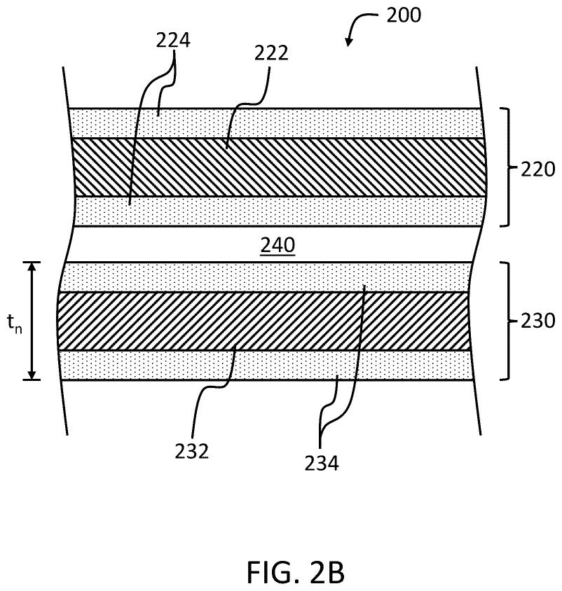

FIGS. 2A and 2B are schematic cross-sectional views of a portion of a battery or cell 200 according to illustrative embodiments that includes at least one positive electrode 220 and at least one negative electrode 230. The size, shape, and configuration of the battery may be selected based on the desired application or other considerations. For example, the electrodes may be flat plate electrodes, wound electrodes (e.g., in a jellyroll, folded, or other configuration), or folded electrodes (e.g., Z-fold electrodes). According to other illustrative embodiments, the battery may be a button cell battery, a thin film solid state battery, or another suitable type of lithium-ion battery.

A separator 240 is provided intermediate, or between, the positive electrode 220 and the negative electrode 230. The separator 240 may be, or may include, any suitable material or combination of materials. Suitable separator materials may include, for example, a polymeric material, such as a polypropylene/polyethylene copolymer or another polyolefin multilayer laminate including micropores formed therein to allow electrolyte lithium ions to flow from one side of the separator to the other.

The negative electrode 230 includes a negative electrode current collector 232. The negative electrode current collector 232 may be, or include, any suitable material and, in particular, any suitable conductive material, such as copper, a copper alloy, titanium, a titanium alloy, aluminum, or an aluminum alloy. In some embodiments, the negative electrode current collector 232 may be, or include, a foil, such as aluminum foil, an aluminum alloy foil, a titanium foil, a titanium alloy foil, a copper foil, or a copper alloy foil. Negative electrode current collectors that are, or include, a foil may advantageously enable a greater ratio of negative electrode active material to total negative electrode material (e.g., by weight, by volume, etc.). Negative electrode current collectors that are, or include, aluminum/aluminum alloy may be relatively inexpensive, easily formed into a current collector, electrically conductive, readily weldable, corrosion resistant, and generally commercially available. Additionally, negative electrode current collectors that are, or include aluminum/aluminum alloy may advantageously have a relatively low density, which enables a greater ratio of negative electrode active material to total negative electrode material, particularly by weight. It will be understood in light of this disclosure that any suitable negative electrode current collector materials, or combination of materials, may be used, and the disclosure is not limited in this regard. It will be further understood in light of the present disclosure that suitable negative electrode current collector materials, or combination of materials, may be selected based on factors, such as those described herein

While the negative electrode current collector 232 is illustrated and described herein as being, or including, a thin foil material, each negative current collector may have any of a variety of other suitable configurations. Suitable negative current collector configurations may include, for example, a grid (e.g., a mesh grid), an expanded metal grid, a photochemically etched grid, or a metallized polymer film.

In some embodiments, suitable negative electrode current collector materials (e.g., materials of the negative electrode current collector 232) may be selected based on first-cycle irreversible capacity of the negative electrode and/or first-cycle irreversible capacity of the positive electrode. An electrode's irreversible capacity may be described as the decrease in capacity (such as specific capacity, e.g., in mAh/g) that occurs between formation and the first charge/discharge cycle. In other words, irreversible capacity of an electrode may be described as the difference between the electrode's capacity when formation is complete and the electrode's capacity after the first discharge and recharge. Irreversible capacity of an electrode active material may be determined, or measured using any suitable method or technique, such as charge and discharge studies in a half cell with a lithium counter electrode using, e.g., a potentiostat or galvanostat with potential and current controls or on battery cycling equipment with current and potential controls, as just two examples. Additionally or alternatively, published values may be available. In certain embodiments, the negative electrode first-cycle irreversible capacity may be increased during cell formation, for example, by lithiating the negative electrode to a lower potential.

As an example, suitable negative electrode current collector materials may be, or include, copper when the negative electrode first-cycle irreversible capacity is less than the positive electrode first-cycle irreversible capacity (e.g., to enable a cell capable of deep discharge and/or zero-volt tolerance). As another example, suitable negative electrode current collector materials may be, or include, aluminum when the negative electrode first-cycle irreversible capacity is greater than the positive electrode first-cycle irreversible capacity.

The negative electrode 230 further includes a negative electrode active material 234 disposed (e.g., deposited, coated, etc.) on the negative electrode current collector 232. While FIG. 2A shows the negative electrode active material 234 provided on only one side of the negative electrode current collector 232, a layer of active material similar or identical to the negative electrode active material 234 may be provided (e.g., coated) on both sides of the negative electrode current collector 232, for example, as shown in FIG. 2B.

The negative electrode active material 234 may be, or may include, any suitable materials. Suitable negative electrode materials may be selected based on factors such as energy density, interfacial kinetics, electrical conductivity, lithium-ion diffusivity, particle size, particle surface area, density, porosity, and tortuosity, as examples. In certain embodiments, the negative electrode active material 234 is, or includes, a niobium-based oxide, or niobium-based negative electrode active material. As used herein, the term “niobium-based oxide” refers to a Niobium-containing metal oxide wherein Niobium or another metal is oxidized. Any suitable niobium-based oxides may be used. Suitable niobium-based oxides may be, or include, for example, Nb₂O₅, a Nb—Ti—O oxide, a Nb—W—O oxide, a Nb—Ti—W—O oxide, a doped Ti—Nb—O oxide, or a combination of two or more thereof. As another example, the niobium-based oxide may be represented by the formula Nb_{1-x-y}TiₓW_{y}O_{z}, as discussed herein. As yet another example, the niobium-based oxide may be, or include, titanium-niobium-tungsten negative electrode composites, as discussed herein, which may include a pseudoternary composition with a metal portion comprising a niobium fraction, a titanium fraction, and a tungsten fraction. In one or more such embodiments, the pseudoternary composition is represented by the formula Nb_{1-x-y} TiₓW_{y}O_{z}. As still another example, the niobium-based oxide may be, or include, a metal portion and an oxygen portion, with the metal portion including a titanium fraction, a niobium fraction, and a doped fraction having a dopant, where the metal portion comprises 5 atom % of the doped metal fraction. In one or more such embodiments, the doped metal fraction is, or includes, rhenium, cerium, praseodymium, neodymium, promethium, samarium, europium, gadolinium, dysprosium, holmium, erbium, thulium, ytterbium, or a combination of two or more thereof.

The negative electrode active material 234 may include one or more additional suitable negative electrode active materials, such as lithium-titanium-oxide (LTO, represented by the formula Li₄Ti₅O₁₂), graphite, lithium, lithium-alloying materials, intermetallic materials (e.g., alloys), and silicon, as examples. In some embodiments, materials such as binders and/or electrically conductive additives (e.g., a carbon coating) may be utilized in conjunction with, or within, the layer of the negative electrode active material 234, for example, to bond, or hold, the various negative electrode components together.

In some embodiments, negative electrode active materials (e.g., the negative electrode active material 234) may be selected or characterized based on the material's lithiation potential compared to Li+/Li. A material's lithiation potential compared to Li+/Li may be determined, or measured, using any suitable method or technique, such as charge and discharge studies in a half cell with a lithium counter electrode using, e.g., a potentiostat or galvanostat with potential and current controls or on battery cycling equipment with current and potential controls, as just two examples. Suitable negative electrode active material lithiation potentials may be selected based on factors such as desired operating voltage range of the cell (e.g., the cell 200), relative electrode operating potential of the positive electrode active material, or capability to maintain solid electrolyte interface (SEI) by minimizing electrolyte decomposition reactions, as examples. As another example, suitable negative electrode active material lithiation potentials may be selected based on providing a cell with an operating voltage range that is sufficiently different from the lithium plating potential. Suitable negative electrode active material lithiation potentials compared to Li+/Li may be, or include, between 0.5 V (volts) and 3.5 V, or between 1 V and 3 V, for example. As further examples, suitable negative electrode active material lithiation potentials compared to Li+/Li may be 0.5 V or greater, 0.7 V or greater, 1 V or greater, 1.2 V or greater, 1.5 V or greater, 1.7 V or greater, 2 V or greater, 2.2 V or greater, 2.5 V or greater, 2.7 V or greater, 3 V or greater, 3.2 V or greater, or 3.5 V or greater, and/or 3.5 V or less, 3.3 V or less, 3 V or less, 2.8 V or less, 2.5 V or less, 2.3 V or less, 2 V or less, 1.8 V or less, 1.5 V or less, 1.3 V or less, 1 V or less, 0.8 V or less, or 0.5 V or less.

Each of the one or more negative electrodes (e.g., the negative electrode 230) may have any suitable area specific capacity. Area specific capacity of the negative electrode may be defined as the energy density, or capacity, of the negative electrode per unit area of negative electrode active material. The area of the negative electrode active material may be defined as the area of the negative electrode active material on a plane defined by an interface between the negative electrode active material and a respective current collector. Negative electrode area specific capacities may be affected by factors such as negative electrode thickness, negative electrode density, and negative electrode loading, each of which may be described as having a positive correlation to negative electrode area specific capacity. As further examples, negative electrode area specific capacity may be described as positively correlated to negative electrode active material thickness (e.g., deposition thickness of the negative electrode active material on aluminum foil, copper foil, the current collector, etc.), density of the negative electrode, concentration (e.g., weight-percent, volume-percent, etc.) of negative electrode active material in the negative electrode (e.g., percentage of niobium-based oxide in the negative electrode active material coating layer), and active material utilization (i.e., mass specific capacity, measured, for example, in milliamp-hours per gram). Negative electrode area specific capacity may be determined as the product of negative electrode active material loading (i.e., the mass of negative electrode active material per unit area of the negative electrode, for example, measured in g/cm²), content (i.e., the content of the negative electrode active material in the negative electrode active material composition e.g., measured in wt-%), and niobium-based oxide utilization (e.g., measured in mAh/g). In an example, an illustrative negative electrode having a negative electrode active material loading value of 0.012 g/cm², a niobium-based oxide content of 93 wt-%, and a niobium-based oxide utilization of 270 mAh/g is determined to have an area specific capacity of 3 mAh/cm².

While, as described herein, relatively high negative electrode area specific capacity may advantageously afford improved cell specific capacity, high negative electrode area specific capacity is also observed to result in reduced stability, for example, due to limited adhesion between the negative electrode active material and the negative electrode current collector. Suitable negative electrode area specific capacities may be, for example, between 1.5 milliamp-hours per square centimeter (mAh/cm²) and 3 mAh/cm², or between 1 mAh/cm² and 6 mAh/cm². In one embodiment, the negative electrode area specific capacity is approximately 3 mAh/cm². As further examples, suitable negative electrode area specific capacities may include 1 mAh/cm² or greater, 1.5 mAh/cm² or greater, 2 mAh/cm² or greater, 2.5 mAh/cm² or greater, 3 mAh/cm² or greater, 3.5 mAh/cm² or greater, 4 mAh/cm² or greater, 4.5 mAh/cm² or greater, 5 mAh/cm² or greater, 5.5 mAh/cm² or greater, or 6 mAh/cm² or greater, and/or 6 mAh/cm² or less, 5.5 mAh/cm² or less, 5 mAh/cm² or less, 4.5 mAh/cm² or less, 4 mAh/cm² or less, 3.5 mAh/cm² or less, 3 mAh/cm² or less, 2.5 mAh/cm² or less, 2 mAh/cm² or less, 1.5 mAh/cm² or less, or 1 mAh/cm² or less.

The positive electrode 220 includes a positive electrode current collector 222. The positive electrode current collector 222 may be, or may include, any suitable material and, in particular, any suitable conductive material, such as titanium, a titanium alloy, aluminum, or an aluminum alloy. In some embodiments, the positive electrode current collector 222 may be, or may include, a foil, such as aluminum foil or an aluminum alloy foil. Positive electrode current collectors that are, or include, a foil may advantageously enable a greater ratio of positive electrode active material to total positive electrode material (e.g., by weight, by volume, etc.). Positive electrode current collectors that are, or include, aluminum/aluminum alloy may advantageously be relatively inexpensive, easily formed into a current collector, electrically conductive, readily weldable, corrosion resistant, and generally commercially available. Additionally, positive electrode current collectors that are, or include aluminum/aluminum alloy may advantageously have a relatively low density, which enables a greater ratio of positive electrode active material to total positive electrode material, particularly by weight. It will be understood in light of this disclosure that any suitable positive electrode current collector materials, or combination of materials, may be used, and the disclosure is not limited in this regard. It will be further understood in light of the present disclosure that suitable positive electrode current collector materials, or combination of materials, may be selected based on factors, such as those described herein.

While the positive electrode current collector 222 is illustrated and described herein as being, or including, a thin foil material, each positive current collector may have any of a variety of other suitable configurations. Suitable positive current collector configurations may include, for example, a grid (e.g., a mesh grid), an expanded metal grid, a photochemically etched grid, or a metallized polymer film.

The positive electrode 220 further includes a positive electrode active material 224 disposed (e.g., deposited, coated, etc.) on the positive electrode current collector 222. While FIG. 2A shows the positive electrode active material 224 provided on only one side of the positive electrode current collector 222, a layer of active material similar or identical to the positive electrode active material 224 may be provided (e.g., coated) on both sides of the positive electrode current collector 222.

The positive electrode active material 224 may be, or may include, any suitable materials. Suitable positive electrode active materials may be selected based on factors such as energy density, interfacial kinetics, electrical conductivity, lithium-ion diffusivity, particle size, particle surface area, density, porosity, and tortuosity, as examples. In one or more embodiments, the positive electrode active material 224 is a material or compound that includes lithium. In at least one embodiment, the positive electrode active material 224 is, or includes, a lithium transition metal oxide. For example, the positive electrode active material 224 may be, or include, lithium transition metals such as lithium cobalt oxide (LCO, represented by the formula LiCoO₂), LiCoMnO₄, LiCoPO₄, or LiNi_{0.5}Mn_{1.5}O₄. As further examples, the positive electrode active material 224 may be, or include, a composition represented by the formula LiNiₓMn_{y}Co_{1-x-y}O₂, a composition represented by the formula LiNiₓCo_{y}Al_{1-x-y}O₂, and/or a lithium-rich layered oxide represented by the formula Li₁+xTM₁−xO₂ (where TM represents a transition metal). The lithium included in the positive electrode active material 224 may be doped and undoped during discharging and charging of the battery, respectively. The positive electrode active material 224 may include one or more additional suitable positive electrode active materials, such as lithium-metal oxides (e.g., LiMn₂O₄, Li(NiₓMn_{y}Co_{z})O₂), vanadium oxides, olivines (e.g., LiFePO₄), rechargeable lithium oxides, or manganese dioxide, as examples. Furthermore, the positive electrode active material 224 may include a combination of two or more suitable materials. It will be understood in light of this disclosure that any suitable positive electrode active materials, or combination of materials, may be used, and the disclosure is not limited in this regard. It will be further understood in light of the present disclosure that suitable positive electrode active materials, or combination of materials, may be selected based on factors, such as those described herein.

In some embodiments, materials such as binders and conductive additives may be utilized in conjunction with, or within, the layer of the positive electrode active material 226, for example, to bond, or hold, the various positive electrode components together. For example, a layer of coating material including the positive electrode active material (e.g., LCO) may include one or more suitable additives, such as suitable conductive additives and suitable binders. Suitable conductive additives may include, for example, one or more conductive carbon agents, such as graphite, carbon black and/or carbon nanotubes. Suitable binders may include, for example, polyvinylidene fluoride (PVDF) and/or an elastomeric polymer. It will be understood in light of this disclosure that any suitable binder and/or conductive additive materials, or combination of materials, may be used, and the disclosure is not limited in this regard. It will be further understood in light of the present disclosure that suitable binder and/or conductive additive materials, or combination of materials, may be selected based on factors, such as those described herein.

In some embodiments, positive electrode active materials (e.g., the positive electrode active material 224) may be selected or characterized based on the material's operating electrode potential compared to Li+/Li. A material's operating electrode potential compared to Li+/Li may be determined, or measured, using any suitable method or technique, such as charge and discharge studies in a half cell with a lithium counter electrode using, e.g., a potentiostat or galvanostat with potential and current controls or on battery cycling equipment with current and potential controls, as just two examples. Suitable positive electrode active material operating electrode potentials may be selected based on factors such as desired operating voltage range of the cell (e.g., the cell 200), relative lithiation potential of the negative electrode active material or capability to maintain solid electrolyte interface (SEI) by minimizing electrolyte decomposition reactions, as examples. As another example, suitable negative electrode active material lithiation potentials may be selected based on providing a cell with an operating voltage range that is sufficiently different from the lithium plating potential. Suitable positive electrode active material operating electrode potentials compared to Li+/Li may be, or include, between 2.8 V and 5 V, or between 3 V and 4.5 V, for example. As further examples, suitable positive electrode active material operating electrode potentials compared to Li+/Li may be 3.5 V or greater, 3.8 V or greater, 4 V or greater, 4.3 V or greater, 4.5 V or greater, 4.8 V or greater, or 5 V or greater, and/or 5 V or less, 4.8 V or less, 4.5 V or less, 4.2 V or less, 4 V or less, 3.8 V or less, or 3.5 V or less. It will be understood in light of this disclosure that any suitable positive electrode active material operating electrode potentials compared to Li+/Li may be used, and the disclosure is not limited in this regard. It will be further understood in light of the present disclosure that suitable positive electrode active material operating electrode potentials may be selected based on factors, such as those described herein.

Each of the one or more positive electrodes (e.g., the positive electrode 220) may have any suitable area specific capacity. Area specific capacity of the positive electrode may be defined as the energy density, or specific capacity, of the positive electrode per unit area of positive electrode active material. The area of the positive electrode active material may be defined as the area of the positive electrode active material on a plane defined by an interface between the positive electrode active material and a respective current collector. Positive electrode area specific capacities may be affected by factors such as positive electrode thickness, positive electrode density, and positive electrode loading, each of which may be described as having a positive correlation to positive electrode area specific capacity. As further examples, positive electrode area specific capacity maybe described as positively correlated to positive electrode active material thickness (e.g., deposition thickness of the positive electrode active material on the current collector), density of the positive electrode, concentration (e.g., weight-percent, volume-percent, etc.) of positive electrode active material in the positive electrode (e.g., percentage of lithium transition metal oxide in the positive electrode active material coating layer), and active material utilization (i.e., mass specific capacity, measured, for example, in milliamp-hours per gram). Positive electrode area specific capacity may be determined as the product of positive electrode active material loading (i.e., the mass of positive electrode active material per unit area of the positive electrode, for example, measured in g/cm²), content (i.e., the content of the positive electrode active material in the positive electrode active material composition e.g., measured in wt-%), and lithium transition metal oxide utilization (e.g., measured in mAh/g). Suitable positive electrode area specific capacities may be, for example, between 1.5 milliamp-hours per square centimeter (mAh/cm²) and 3 mAh/cm², or between 1 mAh/cm² and 6 mAh/cm². In one embodiment, the positive electrode area specific capacity is approximately 3 mAh/cm². As further examples, suitable positive electrode area specific capacities may include 1 mAh/cm² or greater, 1.5 mAh/cm² or greater, 2 mAh/cm² or greater, 2.5 mAh/cm² or greater, 3 mAh/cm² or greater, 3.5 mAh/cm² or greater, 4 mAh/cm² or greater, 4.5 mAh/cm² or greater, 5 mAh/cm² or greater, 5.5 mAh/cm² or greater, or 6 mAh/cm² or greater, and/or 6 mAh/cm² or less, 5.5 mAh/cm² or less, 5 mAh/cm² or less, 4.5 mAh/cm² or less, 4 mAh/cm² or less, 3.5 mAh/cm² or less, 3 mAh/cm² or less, 2.5 mAh/cm² or less, 2 mAh/cm² or less, 1.5 mAh/cm² or less, or 1 mAh/cm² or less. It will be understood in light of this disclosure that any suitable positive electrode area specific capacities may be used, and the disclosure is not limited in this regard. It will be further understood in light of the present disclosure that suitable positive electrode area specific capacities may be selected based on factors, such as those described herein.

Electrochemical cells constructed according to illustrative embodiments and preferred ranges described herein may be designed with any suitable operating voltages. The operating voltages of a cell (e.g., the cell 200) may be described as the range of voltages across a positive electrode (e.g., the positive electrode 220) and a negative electrode (e.g., the negative electrode 230) during a cell's charge and discharge cycles (i.e., the range of voltages across the cell when the cell is between 0% state-of-charge and 100% state-of-charge). In certain embodiments, cells constructed according to illustrative embodiments and preferred ranges described herein have operating voltages desirable for use in implantable medical devices such as pacemakers, insulin pumps, cardioverter-defibrillators, drug delivery pumps, and neurostimulators. Desirable operating voltages for use in implantable medical devices include a lower voltage that is sufficiently high to support high-power functions (e.g., Bluetooth low-energy communications) and an upper voltage that is sufficiently low to be supported by the upper operating voltage limit of the electronics included in the device. Suitable operating voltages may be, or include, 1.5 V to 4 V or 1.8 V to 3.4 V, as examples. Additionally or alternatively, cells constructed according to illustrative embodiments and preferred ranges described herein may have any suitable lower operating voltage (i.e., the voltage across the cell when the state-of-charge is 0%). Suitable lower operating voltages may be, or include, 1.5 V or greater, 1.6 V or greater, 1.7 V or greater, 1.8 V or greater, 1.9 V or greater, or 2 V or greater, and/or 2 V or less, 1.9 V or less, 1.8 V or less, 1.7 V or less, 1.6 V or less, or 1.5 V or less. Additionally or alternatively, cells constructed according to illustrative embodiments and preferred ranges described herein may have any suitable upper operating voltage (i.e., the voltage across the cell when the state-of-charge is 100%). Suitable upper operating voltages may be, or include 3.4 V or greater, 3.5 V or greater, 3.6 V or greater, 3.7 V or greater, 3.8 V or greater, 3.9 V or greater, or 4 V or greater, and/or 4 V or less, 3.9 V or less, 3.8 V or less, 3.7 V or less, 3.6 V or less, 3.5 V or less, or 3.4 V or less. It will be understood in light of this disclosure that any suitable operating voltages, upper operating voltages, and/or lower operating voltages may be used, and the disclosure is not limited in this regard. It will be further understood in light of the present disclosure that suitable operating voltages, upper operating voltages, and/or lower operating voltages may be selected based on factors, such as those described herein.

Illustrative cells described herein (e.g., the cell 200) may have any suitable N/P capacity ratio of the negative electrode to the positive electrode. The N/P capacity ratio may be defined as a ratio of the specific capacity, or energy capacity, of the negative electrode to the specific capacity, or energy capacity, of the positive electrode. Suitable N/P capacity ratios may be selected based on factors such as desired cell energy density, desired service life, or desired power capability, as examples. Furthermore, N/P capacity ratios may be affected by factors such as electrode area specific capacity and electrode surface area.

Suitable N/P capacity ratios may be selected based on properties of the negative electrode(s) (e.g., the negative electrode 230) and properties of the positive electrode(s) (e.g., the positive electrode 220). In one or more embodiments, suitable N/P capacity ratios may be selected based on the negative electrode capacity fade rate compared with the positive electrode capacity fade rate. Electrode capacity fade may be described as the rate (e.g., per charge/discharge cycle or per mAh discharged) at which the capacity (such as specific capacity, e.g., in mAh/g) of an electrode is reduced compared with the initial capacity of the electrode. Electrode capacity fade rate may be affected by factors such as electrode active materials composition (e.g., material composition of the negative electrode active material 234 or material composition of the positive electrode active material 224) and the operating voltage range of the cell (e.g., the cell 200), as examples. An electrode's capacity fade may be determined, or measured, using any suitable method or technique, such as charge and discharge studies in a half cell with a lithium counter electrode using, e.g., a potentiostat or galvanostat with potential and current controls or on battery cycling equipment with current and potential controls and determining the rate at which capacity (i.e., lithiation or delithiation) changes as a function of elapsed cycles or elapsed time, for example. In one or more embodiments, an electrode capacity fade rate may be determined, or characterized, as a capacity fade (e.g., in mAh/g) over a number of charge/discharge cycles. For example, electrode capacity fade rate may be determined, or characterized, as a capacity fade over the first 5, 10, 25, 50, or 100 cycles, as just a few examples.

Additionally or alternatively, suitable N/P capacity ratios may be selected based on the negative electrode impedance growth rate compared with the positive electrode impedance growth rate. Over a cell's life time (e.g., over the course of charge/discharge cycles), an electrode's impedance tends to increase. The electrode's impedance growth is correlated to capacity fade and may be used to measure, or determine, capacity fade. Negative electrode impedance growth may be determined, or measured, using any suitable method or technique, such as by measuring pulse resistance at multiple depths of discharge in a charge/discharge cycle test of a half cell with a lithium counter electrode, and determining the rate at which the pulse resistance changes with elapsed cycles or elapsed time, for example. As another example, resistance growth can be inferred via charge/discharge studies in a half cell with a lithium counter electrode at multiple rates, determining the capacity delivered at the highest rate compared with the slowest rate, and determining changes in the ratio with elapsed cycles or elapsed time.

For example, suitable N/P capacity ratios may be less than 1 (i.e., a “negative limited” cell, where the negative electrode capacity is less than the positive electrode capacity) when the negative electrode capacity fade is less than the positive electrode capacity fade. In other words, in one or more embodiments, the cell has a N/P ratio of less than 1 and a positive electrode fade greater than a negative electrode fade.

As another example, suitable N/P capacity ratios may be greater than 1 (i.e., a “positive limited” cell, where the positive electrode capacity is less than the negative electrode capacity) when the negative electrode capacity fade is greater than the positive electrode capacity fade. In other words, in one or more embodiments, the cell has a N/P ratio of greater than 1 and a positive electrode fade less than a negative electrode fade.

In one or more embodiments, illustrative cells described herein may be arranged with line-to-line alignment between the negative electrode (e.g., the negative electrode 230) and the positive electrode (e.g., the positive electrode 220). As used herein, the term “line-to-line” describes a relationship between two elements such that edges or perimeters of corresponding surfaces of the two elements are substantially aligned. In other words, when two elements are arranged line-to-line with each other, one of the two elements may not overhang the other of the two elements by more than a threshold tolerance. In general, during fabrication or manufacture of devices and apparatus, the physical characteristics (e.g., height, width, position, etc.) of such devices and apparatus are subject to variation within a tolerance. Accordingly, a width of any overhang between two elements arranged line-to-line may be no greater than an acceptable threshold tolerance. Acceptable threshold tolerances of an overhang width between two elements arranged line-to-line may be, for example, 0.5 mm or less, 0.4 mm or less, 0.3 mm or less, 0.2 mm or less, or 0.1 mm or less. Line-to-line arrangement of the electrodes may advantageously afford improved energy densities (e.g., by mechanically improving package efficiency of the assembled cell) and reduced risk of lithium sequestration in the overhang regions during cycling or storage. Furthermore, line-to-line arrangement of the electrodes may advantageously reduce risk of telescoping during cell assembly.

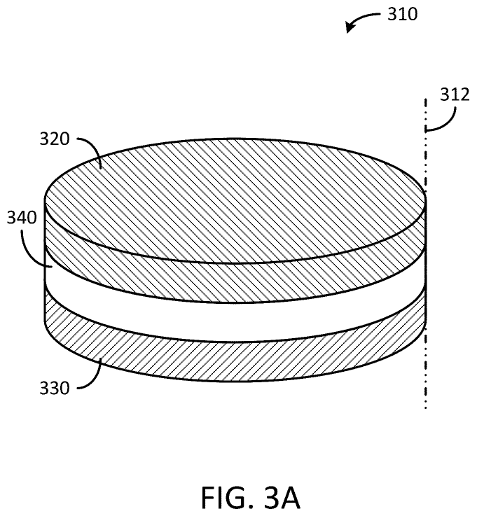

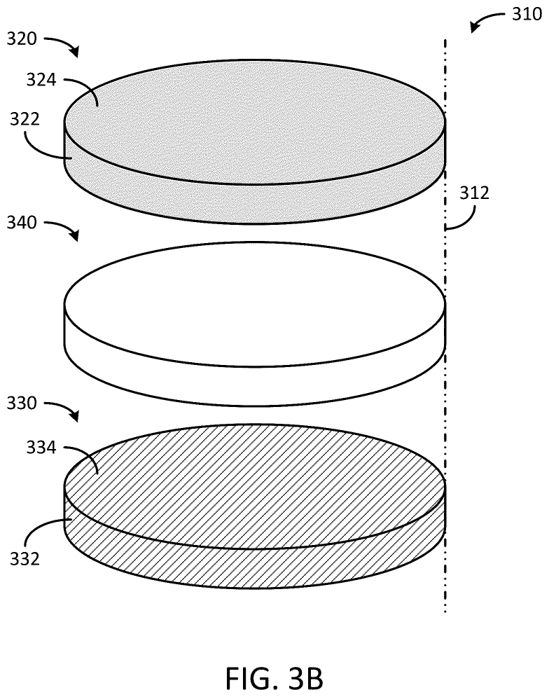

A perspective view of an illustrative electrode stack 310 that includes electrodes arranged line-to-line is shown in FIG. 3A. An exploded perspective view of the illustrative electrode stack 310 of FIG. 3A is shown in FIG. 3B. The illustrative electrode stack 310 includes a positive electrode 320, a negative electrode 330, and a separator 340. The positive electrode 320 may include positive electrode major surfaces 324 and one or more positive electrode edges 322 defining a perimeter of the cathode major surfaces 324. Similarly, the negative electrode 330 may include negative electrode major surfaces 334 and one or more negative electrode edges 332 defining a perimeter of the negative electrode major surfaces 334. In other words, the positive electrode 320 and the negative electrode 330 define flat sheets with the positive electrode major surfaces 324 and the negative electrode major surfaces 334 may be arranged parallel to one another. In general, at least one of the negative electrode major surfaces 334 may face one of the positive electrode major surfaces 324. Furthermore, the one or more negative electrode edges 332 may be arranged line-to-line with the one or more positive electrode edges 322. In other words, a line 312 that is orthogonal to the negative electrode major surfaces 334 and coextensive with the one or more negative electrode edges 332 may also be orthogonal to the positive electrode major surfaces 324 and coextensive with the one or more positive electrode edges 322. While manufacturing tolerances may result in a slight overhang of one or both of the positive electrode 320 and the negative electrode 330, any overhang of the positive electrode 320 and the negative electrode 330 may have an acceptable maximum width and still be described as being arranged line-to-line. Acceptable maximum line-to-line overhang widths may be, for example, 0.5 mm or less, 0.4 mm or less, 0.3 mm or less, 0.2 mm or less, or 0.1 mm or less.

While shown as elliptically shaped plates, the positive electrode 320 and the negative electrode 330 in a line-to-line arrangement may have any suitable shape. In general, the shape of plate electrodes such as the positive electrode 320 and the negative electrode 330 may be defined by the shape of their respective major surfaces 324, 334. The positive electrode major surfaces 324 and the negative electrode major surfaces 334 may take on any suitable two-dimensional shape such as, for example, an ellipse, a polygon, a shape with multiple curved sides (e.g., a circular triangle), a shape with curved and straight sides (e.g., a circular segment, a circular sector, a stadium, etc.), etc. In general, the positive electrode major surfaces 324 and the negative electrode major surfaces 334 may be substantially the same shape and have substantially the same dimensions to allow the positive electrode edges 322 and the negative electrode edges 332 to be arranged line-to-line. Furthermore, while the illustrative electrode stack 310 is shown as a stacked plate arrangement, electrode stacks, or cells, with line-to-line electrodes can be any suitable electrode arrangement. For example, electrode stacks, or cells, that include line-to-line electrodes may include a coiled electrode arrangement, a cylindrical and coiled electrode arrangement, or any other electrode arrangement where edges of the electrode can be aligned.

One issue associated with lithium-ion batteries constructed outside the preferred ranges described herein relates to the ability of such batteries to withstand repeated charge cycling that involves discharges to near-zero-volt conditions (so-called “deep discharge” conditions). This deep discharge cycling may decrease the attainable full charge capacity of the batteries (i.e., capacity fade). For example, a battery constructed outside the preferred ranges described herein that initially is charged to 2.8 V may experience capacity fade with repeated deep discharge cycling such that after 150 cycles the full charge capacity of the battery is much less than the initial capacity.

In one or more embodiments, illustrative cells described herein may be designed, or configured, to be tolerant to deep discharge (e.g., able to undergo deep discharge with increased resistance to capacity fade compared with lithium-ion batteries constructed outside the preferred ranges described herein), or deep discharge tolerant. In certain embodiments, illustrative cells described herein may be designed, or configured, to be zero-volt tolerant. As used herein, the term “zero-volt tolerant” describes a cell that able to undergo discharge to an inert state (that is, discharge such that the voltage across the electrodes is zero) with increased resistance to capacity fade compared with lithium-ion batteries constructed outside the preferred ranges described herein.

Zero-volt tolerance (and/or deep discharge tolerance) may be affected by factors such as electrode irreversible capacity, cell operating voltage, negative electrode lithiation potential (compared with Li/Li+), negative electrode lithiation potential when the cell voltage is zero (i.e., negative electrode zero crossing potential, or the negative electrode lithiation potential when the negative electrode is at near full delithiation), positive electrode operating potential (compared with Li/Li+), positive electrode operating potential when the cell voltage is zero (i.e., positive electrode zero crossing potential, or the positive electrode operating potential when the positive electrode is at near full lithiation), negative electrode current collector materials, or positive electrode current collector materials, as some examples.

As described herein, negative electrode active materials (e.g., the negative electrode active material 234) may be selected or characterized based on the material's lithiation potential compared to Li+/Li. Additionally or alternatively, in illustrative cells including deep discharge tolerance or zero-volt tolerance, suitable negative electrode active materials may be selected or characterized based on the material's potential at near full delithiation. As used herein, near full delithiation may be, or refer to 95% delithiation or greater, 96% delithiation or greater, 97% delithiation or greater, 98% delithiation or greater, or 99% delithiation or greater. Negative electrode active material delithiation may be measured, or determined, using any suitable method or technique, such as by using a reference electrode to measure the potential of the negative electrode relative to the reference electrode, for example. As another example, negative electrode active material delithiation may be estimated using voltage curve models. In certain embodiments, suitable negative electrode active material lithiation potentials at 95% delithiation are relatively high. In other words, suitable negative electrode active materials may have relatively high zero-crossing potentials. As an example, suitable negative electrode active material lithiation potentials at 95% delithiation may be, or include, 3.5 V or greater. As further examples, suitable negative electrode active material lithiation potentials at 95% delithiation may be, or include 2.8 V or greater, 2.9 V or greater, 3 V or greater, 3.1 V or greater, 3.2 V or greater, 3.3 V or greater, 3.4 V or greater, or 3.6 V or greater.

In one or more embodiments, suitable negative electrode active material potentials at deep discharge condition (e.g., zero-volt condition) may be selected based on the negative electrode first-cycle irreversible capacity and/or positive electrode first-cycle irreversible capacity. More particularly, suitable negative electrode active material lithiation potentials at deep discharge condition (e.g., zero-volt condition) may be selected based on the relative first-cycle irreversible capacity value of the negative electrode compared with that of the positive electrode. In certain embodiments, when the negative electrode first-cycle irreversible capacity is greater than the positive electrode first-cycle irreversible capacity, suitable negative electrode active material lithiation potentials at deep discharge condition (e.g., zero-volt condition) are relatively high. As an example, when the negative electrode first-cycle irreversible capacity is greater than the positive electrode first-cycle irreversible capacity, suitable negative electrode active material lithiation potentials at deep discharge condition (e.g., zero-volt condition) and at 95% delithiation may be, or include 3.5 V or greater.

As described herein, positive electrode active materials (e.g., the positive electrode active material 224) may be selected or characterized based on the material's operating potential compared to Li+/Li. Additionally or alternatively, in illustrative cells including deep discharge tolerance or zero-volt tolerance, suitable positive electrode active materials may be selected or characterized based on the material's operating potential at near full lithiation. As used herein, near full lithiation may be, or refer to 95% lithiation or greater, 96% lithiation or greater, 97% lithiation or greater, 98% lithiation or greater, or 99% lithiation or greater. Positive electrode active material lithiation may be measured, or determined, using any suitable method or technique, such as by using a reference electrode to measure the potential of the positive electrode relative to the reference electrode, for example. As another example, negative electrode active material delithiation may be estimated using voltage curve models. In certain embodiments, suitable positive electrode active material operating potentials at 95% lithiation are relatively low. In other words, suitable positive electrode active materials may have relatively low zero-crossing potentials. As an example, suitable positive electrode active material operating potentials at 95% lithiation may be, or include, 2 V or less. As further examples, suitable positive electrode active material operating potentials at 95% lithiation may be, or include 3.2 V or less, 3.1 V or less, 3 V or less, 2.9 V or less, 2.8 V or less, 2.7 V or less, 2.6 V or less, 2.5 V or less, 2.4 V or less, 2.3 V or less, 2.2 V or less, 2.1 V or less, or 1.9 V or less.

In at least one embodiment, suitable positive electrode active material operating potentials may be selected based on the negative electrode first-cycle irreversible capacity and/or positive electrode first-cycle irreversible capacity. More particularly, suitable positive electrode active material operating potentials at near full lithiation may be selected based on the comparative first-cycle irreversible capacity values of the negative electrode and the positive electrode. In certain embodiments, when the negative electrode first-cycle irreversible capacity is less than the positive electrode first-cycle irreversible capacity, suitable positive electrode active material operating potentials at near full lithiation are relatively low. As an example, when the negative electrode first-cycle irreversible capacity is less than the positive electrode first-cycle irreversible capacity, suitable positive electrode active material operating potentials at 95% lithiation may be, or include, 3 V or less.

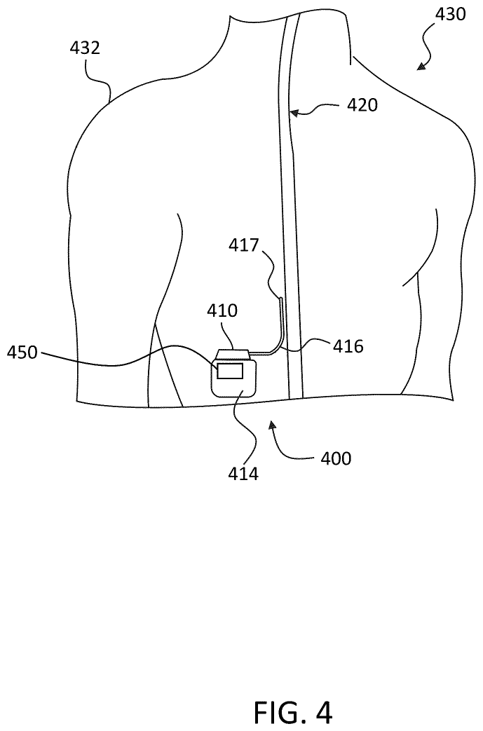

Referring now to FIG. 4, an example of a system 400 (e.g., an implantable medical device) implanted within a body or torso 432 of a patient 430 is shown. The system 400 includes a device 410 in the form of an implantable medical device that for purposes of illustration is shown as a neurostimulator configured to provide a therapeutic treatment for the patient 430.

The device 410 includes a container or housing 414 that is hermetically sealed and biologically inert according to an exemplary embodiment. The container may be made of a conductive material. One or more leads 416 electrically connect the device 410 to a target neurological structure, such as nerve fibers near the patient's spinal column 420. Electrodes 417 are provided to sense electrical activity (e.g., nerve activity) and/or provide electrical stimulation to the neurological structure. At least a portion of the leads 416 (e.g., an end portion of the leads shown as exposed electrodes 417) may be provided adjacent or in contact with the target neurological structure.

The device 410 includes a battery 450 provided therein to provide power for the device 410. The battery 450 is a battery as described herein, and may include an electrochemical cell as described herein, may include an electrode as described herein, or may include a negative electrode as described herein. It should be understood in light of this disclosure that the batteries described herein may be used in any suitable device, and it should be appreciated that various aspects and embodiments of the batteries described herein may be particularly advantageously employed with implantable medical devices.

Reference will now be made in greater detail to various embodiments of titanium-niobium-tungsten negative electrode composites, which may be used in illustrative electrochemical cells described herein, and may furthermore be used in lithium-ion batteries or cells not constructed according to the illustrative embodiments and preferred ranges described herein. Titanium-niobium-tungsten negative electrode composites are further described in U.S. Provisional Patent Application Ser. No. 63/541,456 (titled TITANIUM-NIOBIUM-TUNGSTEN-OXIDE NEGATIVE ELECTRODE COMPOSITES), which is incorporated herein by reference in its entirety.

As described herein, compositions in a Nb—Ti—W—O pseudoternary system may be synthesized. The Nb—Ti—W—O pseudoternary system may be described as a family of compositions synthesized from Nb, Ti, and W in a variety of atomic proportions. Each of the compositions in the Nb—Ti—W—O pseudoternary system may be described as a pseudoternary composition. Each of one or more pseudoternary compositions within the Nb—Ti—W—O pseudoternary system may include one or more compounds, each compound including Nb, Ti, and/or W. In other words, each of the one or more compounds in a pseudoternary composition within the Nb—Ti—W—O pseudoternary system may include Nb, Ti, and W; Nb and Ti; Nb and W; Ti and W; Nb without either of Ti or W; Ti without either of Nb or W; or W without either of Nb or Ti. Each of the one or more compounds in a pseudoternary composition within the Nb—Ti—W—O pseudoternary system may additionally include oxygen (O). Oxygen content of a compound in a pseudoternary composition within the Nb—Ti—W—O pseudoternary system may be described as depending on the oxidation state of each of the Nb, Ti, and/or W in the compound.

Various compounds and pseudoternary compositions in the Nb—Ti—W—O pseudoternary system may be useful as negative electrode active materials. More specifically, single-phase solid solution pseudoternary compositions in the Nb—Ti—W—O pseudoternary system may be useful as negative electrode active materials. In particular, pseudoternary compositions represented by the formula Nb_{1-x-y}TiₓW_{y}O_{z} may be synthesized and may be useful as negative electrode active materials.

Suitable negative electrode materials may include pseudoternary compositions in the Ti—Nb—W—O pseudoternary system described herein. For further examples, suitable negative electrode materials may include graphite, lithium titanium oxide, lithium, lithium-alloying materials, intermetallic materials (e.g., alloys), or silicon. In some embodiments, the negative electrode may include a copper foil, which may include a layer of metallic lithium, such as a coating or plating of lithium or of a lithium alloy.

In one or more embodiments, each negative electrode may include one or more negative electrode active material compositions, which may be or include pseudoternary compositions in the Nb—Ti—W—O pseudoternary system. The pseudoternary compositions may be represented by the formula Nb_{1-x-y}TiₓW_{y}O_{z}. In other words, the electrochemical cell may include a negative electrode active material represented by the formula Nb_{1-x-y}TiₓW_{y}O_{z}.

The pseudoternary compositions may define any suitable crystal morphology, which may include any suitable crystal structures. Suitable crystal structures may be selected based on Li conductivity, electronic conductivity, or suitable sites for Li insertion, as examples. Suitable crystal structures may include octahedra centered by Nb and Ti, octahedra centered by Nb and W, octahedra centered by Nb and Ti and W, octahedra centered by W, or tetrahedra centered by W, as examples. It will be understood in light of the present disclosure that any suitable crystal structure may be used, and the disclosure is not limited in this regard. It will be further understood in light of the present disclosure that suitable crystal structures may vary depending on factors, including those described herein.

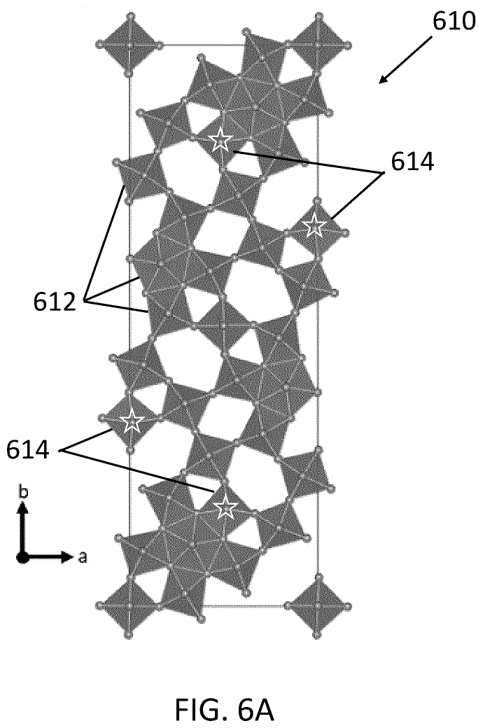

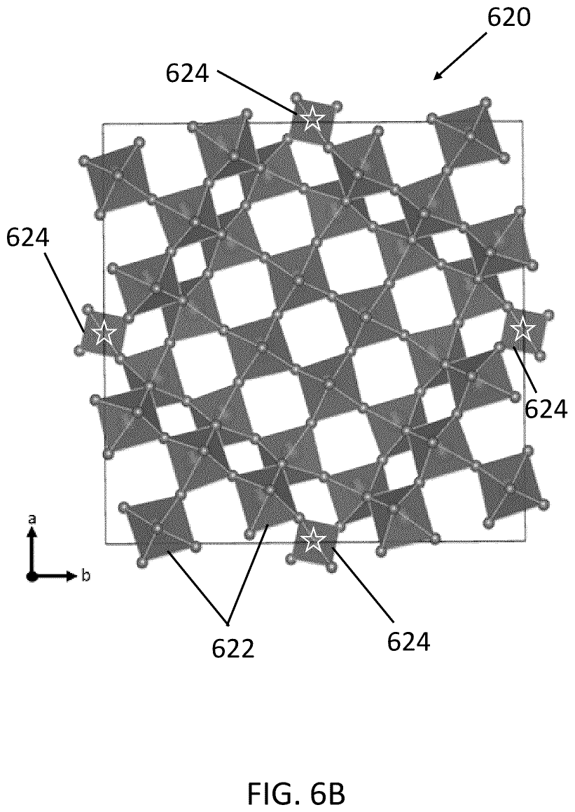

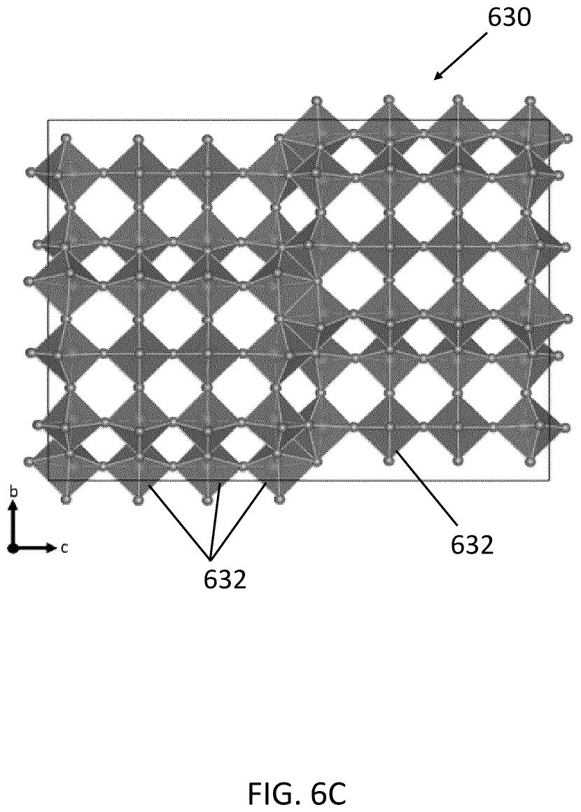

A diagrammatic representation of a suitable crystal structure of an illustrative pseutoternary composition 610 in the Nb—Ti—W—O pseudoternary system is shown in FIG. 6A. The crystal structure of the illustrative pseutoternary composition 610 includes niobium octahedra morphologies 612 centered by a mixture of Nb and Ti; Nb and W; or Nb, Ti, and Nb. The crystal structure of the illustrative pseutoternary composition 610 further includes tungsten octahedra morphologies 614 (identified by a white star) centered by W only.

A diagrammatic representation of another suitable crystal structure of an illustrative pseutoternary composition 620 is shown in FIG. 6B. The crystal structure of the illustrative pseutoternary composition 620 includes niobium octahedra morphologies 622 centered by a mixture of Nb and Ti; Nb and W; or Nb, Ti, and Nb. The crystal structure of the illustrative pseutoternary composition 620 may additionally include tungsten tetrahedra morphologies 624 (identified by a white star) centered by W only.

A diagrammatic representation of still another suitable crystal structure of an illustrative pseutoternary composition 630 is shown in FIG. 6C. The crystal structure of the illustrative pseutoternary composition 630 includes niobium octahedra morphologies 632 centered by a mixture of Nb and Ti; Nb and W; or Nb, Ti, and Nb.

Suitable crystal morphologies may include any suitable average crystal size, or particle size. Suitable crystal sizes may be selected based on desired lifecycle of the electrochemical cell, as an example. Without wishing to be bound by theory, larger crystals, or particles, may be more susceptible to fracture during charge and discharge cycles of an electrochemical cell, which may lead to poor extended cycling. In other words, smaller crystals, or particles, such as approximately 500 nm or smaller, for example, may be generally desirable in order to avoid or reduce cracking, or fracture, of the particles. For further examples, suitable average crystal sizes may be selected based on material compatibility, desired power density, or desired specific capacity, as a few examples. Without wishing to be bound by theory, larger particles generally afford greater specific capacity, and smaller particles generally afford greater power density. Suitable average crystal lengths may include 0.5 um, as an example. As further examples, suitable average crystal lengths may include between 0.1 um and 5 um or between 0.3 um and 1 um. As still further examples, suitable average crystal lengths may include 7 um or less, 5 um or less, 4 um or less, 3 um or less, 2 um or less, 1 um or less, 0.8 um or less, 0.5 um or less, 0.3 um or less, or 0.1 um or less, and/or 0.05 um or greater, 0.1 um or greater, 0.3 um or greater, 0.5 um or greater, 0.8 um or greater, 1 um or greater, 2 um or greater, 3 um or greater, 4 um or greater, or 5 um or greater. It will be understood in light of the present disclosure that any suitable average crystal lengths may be used, and the disclosure is not limited in this regard. It will be further understood in light of the present disclosure that suitable average crystal lengths may vary depending on factors, including those described herein.

The pseudoternary composition may include metal fractions. In particular, the pseudoternary composition may include each of a niobium metal fraction, a titanium metal fraction, and a tungsten metal fraction. The metal fractions of the pseudoternary composition may be described as the atomic proportions of each of the metals (i.e., Nb, Ti, and W) in the pseudoternary composition relative to one another. Metal fractions may be expressed, for example, as an atomic percent (at. %).

The pseudoternary composition may have any suitable Nb metal fraction. Suitable Nb metal fractions may be selected based on desired discharge capacity, desired first cycle irreversible capacity, and/or desired rate retention, as examples. In particular, pseudoternary compositions with Nb metal fractions of 50 at. % or greater may be useful as a negative electrode active material in electrochemical cells where high discharge capacities are desirable. Likewise, pseudoternary compositions with Nb metal fractions of 50 at. % or greater may be useful as a negative electrode active material in electrochemical cells where low first cycle irreversible capacities are desirable. Similarly, pseudoternary compositions with Nb metal fractions between 25 at. % and 65 at. % may be useful as a negative electrode active material in electrochemical cells where high rate retention is desirable. Suitable Nb metal fractions may be selected based on desired specific capacity, as a further example. Without wishing to be bound by theory, higher Nb metal fractions generally afford higher specific capacities. Suitable Nb metal fractions may include between 25 at. % and 65 at. %, between 30 at. % and 80 at. % or between 45 at. % and 65 at. %, as examples. Further examples of suitable Nb metal fractions may include 20 at. % or greater, 25 at. % or greater, 30 at. % or greater, 35 at. % or greater, 40 at. % or greater, 45 at. % or greater, 50 at. % or greater, 55 at. % or greater, 60 at. % or greater, 70 at. % or greater, or 80 at. % or greater, and/or 90 at. % or less, 80 at. % or less, 70 at. % or less, 60 at. % or less, 55 at. % or less, 50 at. % or less, 45 at. % or less, 40 at. % or less, 35 at. % or less, 30 at. % or less, or 25 at. % or less.

The pseudoternary composition may have any suitable Ti metal fraction. Suitable Ti metal fractions may be selected based on desired specific capacity, for example. Without wishing to be bound by theory, higher Ti metal fractions may increase specific capacity. Suitable Ti metal fractions may additionally or alternatively be selected based on desired discharge rate, as another example. Without wishing to be bound by theory, higher Ti metal fractions may generally afford increased discharge rate. Suitable Ti metal fractions may include between 30 at. % and 50 at. % or between 10 at. % and 60 at. %, for example. As further examples, suitable Ti metal fractions may include 5 at. % or greater, 10 at. % or greater, 20 at. % or greater, 30 at. % or greater, 35 at. % or greater, 40 at. % or greater, 45 at. % or greater, or 50 at. % or greater, and/or 60 at. % or less, 50 at. % or less, 45 at. % or less, 40 at. % or less, 35 at. % or less, 30 at. % or less, 20 at. % or less, or 10 at. % or less. It will be understood in light of the present disclosure that any suitable Ti metal fractions may be used, and the disclosure is not limited in this regard. It will be further understood in light of the present disclosure that suitable Ti metal fractions may vary depending on factors, including those described herein.

The pseudoternary composition may have any suitable W metal fraction. Suitable W metal fractions may be selected based on desired specific capacity, as an example. Without wishing to be bound by theory, higher W metal fractions may generally afford increased specific capacity. As another example, suitable W metal fractions may additionally or alternatively be selected based on desired discharge rate. Without wishing to be bound by theory, higher W metal fractions may generally afford increased discharge rate. Suitable W metal fractions may include between 5 at. % and 75 at. %, between 5 at. % and 25 at. %, or between 30 at. % and 75 at. %, for example. As further examples, suitable W metal fractions may include 5 at. % or greater, 10 at. % or greater, 15 at. % or greater, 20 at. % or greater, 25 at. % or greater, 30 at. % or greater, 35 at. % or greater, 40 at. % or greater, 45 at. % or greater, 55 at. % or greater, 65 at. % or greater, or 75 at. % or greater, and/or 80 at. % or less, 75 at. % or less, 65 at. % or less, 55 at. % or less, 50 at. % or less, 45 at. % or less, 40 at. % or less, 35 at. % or less, 30 at. % or less, 25 at. % or less, 20 at. % or less, 15 at. % or less, 10 at. % or less, or 5 at. % or less. It will be understood in light of the present disclosure that any suitable W metal fractions may be used, and the disclosure is not limited in this regard. It will be further understood in light of the present disclosure that suitable W metal fractions may vary depending on factors, including those described herein.

The pseudoternary composition may have any suitable metal fractions of each of Nb, Ti, and W. Suitable metal fractions may include between 15 at. % Nb and 95 at. % Nb, between 2.5 at. % W and 80 at. % W, and between 2.5 at. % Ti and 60 at. % Ti; between 25 at. % Nb and 60 at. % Nb, between 30 at. % W and 75 at. % W and between 5 at. % Ti and 25 at. % Ti; between 60 at. % Nb and 90 at. % Nb, between 5 at. % W and 20 at. % W, and between 5 at. % Ti and 30 at. % Ti; between 30 at. % Nb and 50 at. % Nb, between 35 at. % W and 65 at. % W, and between 5 at. % Ti and 20 at. % Ti; between 75 at. % Nb and 90 at. % Nb, between 5 at. % W and 20 at. % W, and between 5 at. % Ti and 25 at. % Ti; or between 55 at. % Nb and 90 at. % Nb, between 5 at. % W and 15 at. % W, and between 5 at. % Ti and 35 at. % Ti.

In one embodiment, the metal fractions may be approximately 47.5 at. % Nb, approximately 37.5 at. % W, and approximately 15 at. % Ti. In another embodiment, the metal fractions may be approximately 32.5 at. % Nb, approximately 60 at. % W, and approximately 7.5 at. % Ti. In still another embodiment, the metal fractions may be approximately 85 at. % Nb, approximately 10 at. % W, and approximately 5 at. % Ti. In yet another embodiment, the metal fractions may be approximately 85 at. % Nb, approximately 7.5 at. % W, and approximately 7.5 at. % Ti. In still yet another embodiment, the metal fractions may be approximately 77.5 at. % Nb, approximately 15 at. % W, and approximately 7.5 at. % Ti. In still another embodiment, the metal fractions may be approximately 60 at. % Nb, approximately 10 at. % W, and approximately 30 at. % Ti.

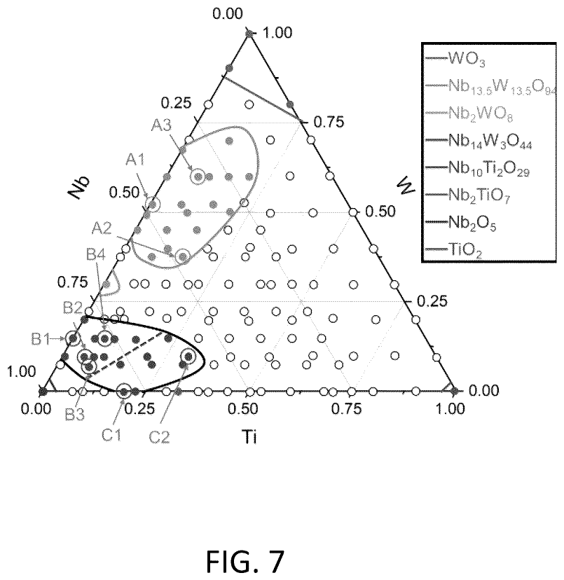

Additionally or alternatively, suitable metal fractions of the pseudoternary composition may have ratios of Nb, Ti, and W within ranges identified in FIG. 7. Such ranges may be identified by the demarked regions containing samples (i) A1, A2, and A3; (ii) B1, B2, B3, and B4; (iii) C1 and C2; or (iv) B1, B2, B3, B4, C1, and C2. Additionally or alternatively, suitable metal fractions of the pseudoternary composition may have ratios of Nb, Ti, and W such that the pseudoternary composition has a crystal structure as indicated in FIG. 6A, 6B, or 6C.

The pseudoternary compositions described herein may be used in negative electrodes. In particular, the pseudoternary compositions described herein may be used in negative electrodes as a negative electrode active material. In some embodiments, negative electrodes including the pseudoternary composition may optionally include a coating layer on the surface of the negative electrode. The coating layer may include any suitable coating material. Suitable coating materials may be selected based on material compatibility, conductivity, density, or stability with an electrolyte, as examples. For example, a relatively thin and dense electrode coating layer may be desirable. Suitable coating materials may include carbon, such as graphite, graphitic carbon, graphene, or carbon black, as a few examples. It will be understood in light of the present disclosure that any suitable coating layers may be used and the disclosure is not limited in this regard. It will be further understood in light of the present disclosure that coating layers may vary depending on factors, including those described herein.

In at least one embodiment, negative electrodes including the pseudoternary composition may be or include an electrode composite, or more specifically, a negative electrode composite. In one or more embodiments, the negative electrode composite may include a negative electrode active material, a binder, and an electrically conductive additive. In particular, the negative electrode active material of the negative electrode composite may include pseudoternary compositions in the Nb—Ti—W—O pseudoternary system, as described herein.

In one or more embodiments, negative electrode composites including the pseudoternary composition may include a binder. The binder may be or include any suitable material or combination of materials. Suitable binder materials may be selected based on material compatibility with the negative electrode active material, material compatibility with the electrically conductive additive, material compatibility with the electrolyte, material compatibility with the separator, material compatibility with other components of the electrochemical cell, density, porosity, tortuosity, elastic modulus, or degree of swelling in electrolyte, as examples. Suitable binder materials may include polymer adhesives, such as polyvinylidene fluoride, as an example. Suitable binder materials may include PTFE (polytetrafluoroethylene), SBR (styrene butadiene rubber), CMC (carboxy methyl cellulose), or polyimides, as further examples. It will be understood in light of the present disclosure that any suitable binder materials may be used, and the disclosure is not limited in this regard. It will be further understood in light of the present disclosure that suitable binder materials may vary depending on factors, including those described herein.

In some embodiments, negative electrode composites including the pseudoternary composition may include an electrically conductive additive. The electrically conductive additive may be or include any suitable material or combination of materials. Suitable electrically conductive additive materials may be selected based on material compatibility with the negative electrode active material, material compatibility with the binder, material compatibility with the electrolyte, material compatibility with the separator, material compatibility with other components of the electrochemical cell, conductivity, particle surface area, density, porosity, tortuosity electronic conductivity, short range electronic conductivity, or long range electronic conductivity, as examples. Suitable electrically conductive additive materials may include carbon black, carbon nanotubes, single wall carbon nanotubes, multiwall carbon nanotubes, graphite, or graphene, as examples. It will be understood in light of the present disclosure that any suitable electrically conductive additive materials may be used, and the disclosure is not limited in this regard. It will be further understood in light of the present disclosure that suitable electrically conductive additive materials may vary depending on factors, including those described herein.

Negative electrode composites including the pseudoternary composition may have any suitable porosity. Porosity may be determined, or measured, using any suitable method or technique, such as Mercury Intrusion Porosimetry or Electromechanical Impedance Spectroscopy, for two examples. Suitable negative electrode composite porosities may be selected based on desired energy density or material properties such as particle size, particle shape, particle surface area, or desired power density, as examples. Without wishing to be bound by theory, higher porosity generally affords higher power. Suitable negative electrode composite porosities may include, for example, about 30%. For further examples, suitable negative electrode composite porosities may include between 10% and 70% or between 15% and 50%. For still further examples, suitable negative electrode composite porosities may include 80% or less, 70% or less, 60% or less, 50% or less, 40% or less, 30% or less, or 20% or less. Suitable negative electrode composite porosities may additionally or alternatively include 5% or greater, 10% or greater, 15% or greater, 20% or greater, 30% or greater, 40% or greater, 50% or greater, 60% or greater, or 70% or greater. It will be understood in light of the present disclosure that any suitable negative electrode composite porosities may be used, and the disclosure is not limited in this regard. It will be further understood in light of the present disclosure that suitable negative electrode composite porosities may vary depending on factors, including those described herein.

Negative electrode composites including the pseudoternary composition may have any suitable surface area. Suitable negative electrode composite surface areas may include, for example, between 0.5 cm² and 300 cm². Further examples of suitable negative electrode composite surface areas may include 0.5 cm² or greater, 1 cm² or greater, 5 cm² or greater, 10 cm² or greater, 25 cm² or greater, 50 cm² or greater, 100 cm² or greater, 150 cm² or greater, 200 cm² or greater, 250 cm² or greater, or 300 cm² or greater, and/or 300 cm² or less, 250 cm² or less, 200 cm² or less, 150 cm² or less, 100 cm² or less, 50 cm² or less, 25 cm² or less, 10 cm² or less, 5 cm² or less, 1 cm² or less, or 0.5 cm² or less. It will be understood in light of the present disclosure that any suitable negative electrode composite surface areas may be used, and the disclosure is not limited in this regard. It will be further understood in light of the present disclosure that suitable negative electrode composite surface areas may vary depending on factors, including those described herein.

The pseudoternary compositions in a Nb—Ti—W—O pseudoternary system described herein—and negative electrodes or negative electrode composites including pseudoternary compositions described herein—may be used in electrochemical cells, such as illustrative electrochemical cells described herein, and may furthermore be used in lithium-ion batteries or cells not constructed according to the illustrative embodiments and preferred ranges described herein.

Electrochemical cells including negative electrodes with the pseudoternary compositions described herein may provide improved specific capacity. In embodiments, such electrochemical cells improve specific capacities at ambient room temperature. In one or more embodiments, such electrochemical cell may have specific capacities at ambient room temperature of between 200 mAh/g and 350 mAh/g or between 230 mAh/g and 320 mAh/g, as examples. In one embodiment, such a cell may have a specific capacity at ambient room temperature of approximately 263 mAh/g. Cell specific capacities at ambient room temperature may additionally or alternatively include 200 mAh/g or greater, 250 mAh/g or greater, 300 mAh/g or greater, or 350 mAh/g or greater, and/or 350 mAh/g or less, 300 mAh/g or less, 250 mAh/g or less, or 200 mAh/g or less.

In some embodiments, electrochemical cells including negative electrodes with the pseudoternary compositions described herein may provide improved specific capacity at an ambient temperature of about 37° C., or about human body temperature. In one or more embodiments, for example, such electrochemical cells may have specific capacities at 37° C. ambient temperature of between 200 mAh/g and 350 mAh/g or between 230 mAh/g and 320 mAh/g, as examples. In one embodiment, such a cell may have a specific capacity at 37° C. ambient temperature of approximately 318 mAh/g. Cell specific capacities at 37° C. ambient temperature may additionally or alternatively include 200 mAh/g or greater, 250 mAh/g or greater, 300 mAh/g or greater, or 350 mAh/g or greater, and/or 350 mAh/g or less, 300 mAh/g or less, 250 mAh/g or less, or 200 mAh/g or less.

As described herein, electrochemical cells including negative electrodes with the pseudoternary compositions may provide improved average discharge voltages, including improved average discharge voltages at ambient room temperature. In some embodiments, for example, such electrochemical cells may have average discharge voltages at ambient room temperature of between 1.55 V and 1.8 V or between 1.4 V and 2 V. In one embodiment, such a cell may have an average discharge voltage at ambient room temperature of approximately 1.7 V. For further examples, such electrochemical cells may have average discharge voltages at ambient room temperature of 1.4 V or greater, 1.5 V or greater, 1.6 V or greater, 1.7 V or greater, 1.8 V or greater, 1.9 V or greater, or 2 V or greater, and/or 2.1 V or less, 2 V or less, 1.9 V or less, 1.8 V or less, 1.7 V or less, 1.6 V or less, or 1.5 V or less.

Additionally or alternatively, electrochemical cells including negative electrodes with the pseudoternary compositions described herein may provide improved average discharge voltages at an ambient temperature of approximately 37° C. In some embodiments, for example, such electrochemical cells may have average discharge voltages at 37° C. ambient temperature of between 1.55 V and 1.8 V or between 1.4 V and 2 V. In one embodiment, such a cell may have an average discharge voltage at 37° C. ambient temperature of approximately 1.6 V. For further examples, such electrochemical cells may have average discharge voltages at 37° C. ambient temperature of 1.4 V or greater, 1.5 V or greater, 1.6 V or greater, 1.7 V or greater, 1.8 V or greater, 1.9 V or greater, or 2 V or greater, and/or 2.1 V or less, 2 V or less, 1.9 V or less, 1.8 V or less, 1.7 V or less, 1.6 V or less, or 1.5 V or less.

As described herein, electrochemical cells including negative electrodes with the pseudoternary compositions may provide improved energy capacity retention after 10 charge-discharge cycles, including improved energy capacity retention after 10 charge-discharge cycles at ambient room temperature. In some embodiments, for example, such electrochemical cells may have energy capacity retentions after 10 charge-discharge cycles at ambient room temperature of between 75% and 99.5% or between 80% and 99%. In one embodiment, such a cell may have an energy capacity retention after 10 charge-discharge cycles at ambient room temperature of approximately 97%. Additionally or alternatively, such electrochemical cells may have energy capacity retentions after 10 charge-discharge cycles at ambient room temperature of 75% or greater, 80% or greater, 85% or greater, 90% or greater, 95% or greater, 97% or greater, 98% or greater, or 99% or greater, and/or 99.5% or less, 99% or less, 98% or less, 97% or less, 95% or less, 90% or less, 85% or less, 80% or less, or 75% or less.

Additionally or alternatively, electrochemical cells including negative electrodes with the pseudoternary compositions described herein may provide improved energy capacity retention after 10 charge-discharge cycles at an ambient temperature of approximately 37° C. In some embodiments, for example, such electrochemical cells may have energy capacity retentions after 10 charge-discharge cycles at 37° C. ambient temperature of between 70% and 99.5% or between 80% and 99%. In one embodiment, such a cell may have an energy capacity retention after 10 charge-discharge cycles at 37° C. ambient temperature of approximately 99%. For further examples, such electrochemical cells may have energy capacity retentions after 10 charge-discharge cycles at 37° C. ambient temperature of 70% or greater, 75% or greater, 80% or greater, 85% or greater, 90% or greater, 95% or greater, 97% or greater, 98% or greater, or 99% or greater, and/or 99.5% or less, 99% or less, 98% or less, 97% or less, 95% or less, 90% or less, 85% or less, 80% or less, or 75% or less.

As described herein, electrochemical cells including negative electrodes with the pseudoternary compositions may provide improved discharge rate retention after 10 charge-discharge cycles, including improved discharge rate retention after 10 charge-discharge cycles at ambient room temperature. In some embodiments, for example, such electrochemical cells may have discharge rate retentions after 10 charge-discharge cycles at ambient room temperature of between 70% and 97% or between 75% and 95%. In one embodiment, such a cell may have a discharge rate retention after 10 charge-discharge cycles at ambient room temperature of approximately 89%. Additionally or alternatively, such electrochemical cells may have discharge rate retentions after 10 charge-discharge cycles at ambient room temperature of 70% or greater, 75% or greater, 80% or greater, 85% or greater, 90% or greater, 95% or greater, or 97% or greater, and/or 99% or less, 97% or less, 95% or less, 90% or less, 85% or less, 80% or less, or 75% or less, or 70% or less.

Additionally or alternatively, electrochemical cells including negative electrodes with the pseudoternary compositions described herein may provide improved discharge rate retention after 10 charge-discharge cycles at an ambient temperature of approximately 37° C. In some embodiments, for example, such electrochemical cells may have discharge rate retentions after 10 charge-discharge cycles at 37° C. ambient temperature of between 70% and 97% or between 80% and 95%. In one embodiment, such a cell may have a discharge rate retention after 10 charge-discharge cycles at 37° C. ambient temperature of approximately 85%. For further examples, such electrochemical cells may have discharge rate retentions after 10 charge-discharge cycles at 37° C. ambient temperature of 70% or greater, 75% or greater, 80% or greater, 85% or greater, 90% or greater, 95% or greater, or 97% or greater, and/or 99% or less, 97% or less, 95% or less, 90% or less, 85% or less, 80% or less, 75% or less, or 70% or less.

As described herein, electrochemical cells including negative electrodes with the pseudoternary compositions may provide improved diffusivity, including improved diffusivity at ambient room temperature. In some embodiments, for example, such electrochemical cells may have diffusivity at ambient room temperature of between 5×10⁻⁸ square centimeters per second (cm²/s) and 5×10⁻⁷ cm²/s or between 7×10⁸ cm²/s and 2×10⁻⁷ cm²/s. In one embodiment, such a cell may have a diffusivity at ambient room temperature of approximately 7.7×10⁻⁸ cm²/s. For further examples, diffusivities at ambient room temperature may include 5×10⁻⁸ cm²/s or greater, 7×10⁻⁸ cm²/s or greater, 9×10⁻⁸ cm²/s or greater, 1×10⁻⁷ cm²/s or greater, 3×10⁻⁷ cm²/s or greater, or 5×10⁻⁷ cm²/s or greater, and/or 5×10⁻⁷ cm²/s or less, 3×10⁻⁷ cm²/s or less, 1×10⁻⁷ cm²/s or less, 9×10⁻⁸ cm²/s or less, 7×10⁻⁸ cm²/s or less, or 5×10⁻⁸ cm²/s or less.

Additionally or alternatively, electrochemical cells including negative electrodes with the pseudoternary compositions described herein may provide improved diffusivity at 37° C. ambient temperature. In some embodiments, for example, such electrochemical cells may have diffusivity at 37° C. ambient temperature of between 5×10⁻⁸ cm²/s and 5×10⁻⁷ cm²/s or between 6×10⁻⁸ cm²/s and 3×10⁻⁷ cm²/s. In one embodiment, such a cell may have a diffusivity at 37° C. ambient temperature of approximately 1.4×10⁻⁸ cm²/s. For further examples, diffusivities at ambient room temperature may include 5×10⁻⁸ cm²/s or greater, 7×10⁻⁸ cm²/s or greater, 9×10⁻⁸ cm²/s or greater, 1×10⁻⁷ cm²/s or greater, 3×10⁻⁷ cm²/s or greater, or 5×10⁻⁷ cm²/s or greater, and/or 5×10⁻⁷ cm²/s or less, 3×10⁻⁷ cm²/s or less, 1×10⁻⁷ cm²/s or less, 9×10⁻⁸ cm²/s or less, 7×10⁻⁸ cm²/s or less, or 5×10⁻⁸ cm²/s or less.

The pseudoternary compositions described herein, and illustrative electrode active materials including the pseudoternary compositions, may be formed using any suitable synthesis method. Suitable synthesis methods to form the electrode active materials including pseudoternary compositions may include solid-state synthesis, sol-gel synthesis, or hydrothermal synthesis, as a few examples.

An illustrative solid-state method of synthesizing negative electrode active materials including a pseudoternary composition may include mixing oxide precursors of each of Nb, W, and Ti, such as oxide precursor powders. The mixing may include any suitable mixing procedure. Suitable mixing procedures may include using a mortar and pestle and/or using a ball mill, for just two examples. Any suitable Nb, W, and Ti oxide precursors may be used. Suitable oxide precursors may include Nb₂O₅ powder, WO₃ powder, and TiO₂ powder, as examples. In some embodiments, suitable solid-state methods of synthesizing such negative electrode active materials may optionally include pelletizing the mixed oxide precursors.

In one or more embodiments, suitable solid-state methods of synthesizing negative electrode active materials including a pseudoternary composition may include calcinating the mixed oxide precursors (e.g., pelletized mixed oxide precursors). The solid-state calcinating may occur at any suitable ambient temperature. Suitable solid-state calcinating ambient temperatures may be selected based on desired crystal structure, for example. In some embodiments, the solid-state calcinating may occur while ramping the ambient temperature, such as ramping the ambient temperature at a heating rate of 10° C. per minute and/or ramping the ambient temperature at a cooling rate of 5° C. per minute, for example. Suitable solid-state calcinating ambient temperatures may include, for example, between 1,000° C. and 2,500° C. For further examples, suitable solid-state calcinating ambient temperatures may include 900° C. or greater, 1,000° C. or greater, 1,300° C. or greater, 1,500° C. or greater, 1,600° C. or greater, 2,000° C. or greater, 2,200° C. or greater, or 2,500° C. or greater, and/or 2,500° C. or less, 2,200° C. or less, 2,000° C. or less, 1,600° C. or less, 1,500° C. or less, 1,300° C. or less, or 1,000° C. or less. It will be understood in light of the present disclosure that any suitable solid-state calcinating ambient temperatures may be used and the disclosure is not limited in this regard. It will be further understood in light of the present disclosure that suitable solid-state calcinating ambient temperatures may vary depending on factors, including those described herein.

The solid-state calcinating may occur for any suitable duration, which may be defined as a time period during which the sample is in a target solid-state calcinating ambient temperature, for example. Suitable solid-state calcinating durations may be selected based on solid-state calcinating ambient temperature, desired degree of crystallinity, or desired crystallite size, for example. Suitable solid-state calcinating durations may include, for example, between 10 hours and 10 days or between 12 hours and 5 days. For further examples, suitable solid-state calcinating durations may include 10 hours or greater, 12 hours or greater, 18 hours or greater, 2 days or greater, 5 days or greater, or 10 days or greater, and/or 10 days or less, 5 days or less, 2 days or less, 18 hours or less, or 12 hours or less. It will be understood in light of the present disclosure that any suitable solid-state calcinating duration may be used and the disclosure is not limited in this regard. It will be further understood in light of the present disclosure that suitable solid-state calcinating duration may vary depending on factors, including those described herein.

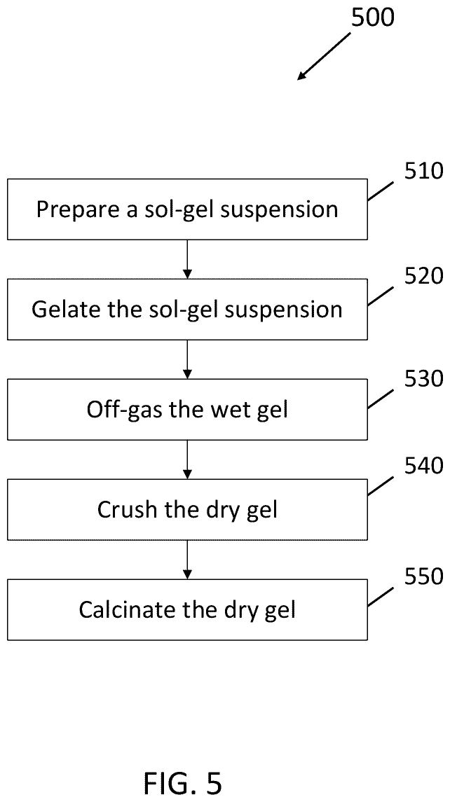

A block diagram of an illustrative sol-gel method 500 of synthesizing negative electrode active materials including a pseudoternary composition is shown in FIG. 5.

It is contemplated that any embodiment discussed in this specification can be implemented with respect to any method or composition of the present disclosure, and vice versa. Furthermore, compositions of the present disclosure can be used to achieve methods of the present disclosure.

In some embodiments, the method 500 may include preparing a sol-gel suspension 510. The sol-gel suspension may include a tungsten compound, a titanium compound, a niobium compound, and a chelating agent.

The sol-gel suspension may include any suitable tungsten compound. Suitable tungsten compounds may be selected based on solubility in desired solvent (e.g., water, alcohol, acid, etc.), or counter ions (e.g., nitrates, acetates, etc., which may decompose during synthesis), for example. Suitable tungsten compounds may include, for example, tungsten trioxide or ammonium metatungstate hydrate, as just two examples. It will be understood in light of the present disclosure that any suitable tungsten compounds may be used, and the disclosure is not limited in this regard. It will be further understood in light of the present disclosure that suitable tungsten compounds may vary depending on factors, including those described herein.

The sol-gel suspension may include any suitable titanium compound. Suitable titanium compounds may be selected based on solubility in desired solvent (e.g., water, alcohol, acid, etc.) or counter ions (e.g., nitrates, acetates, etc., which may decompose during synthesis), for example. Suitable titanium compounds may include titanium butoxide or titanium(IV) bis(ammonium lactato)dihydroxide, as an example. It will be understood in light of the present disclosure that any suitable titanium compounds may be used, and the disclosure is not limited in this regard. It will be further understood in light of the present disclosure that suitable titanium compounds may vary depending on factors, including those described herein.

The sol-gel suspension may include any suitable niobium compound. Suitable niobium compounds may be selected based on solubility in desired solvent (e.g., water, alcohol, acid, etc.), or counter ions (e.g., nitrates, acetates, etc., which may decompose during synthesis), for example. Suitable niobium compounds may include ammonium niobate oxalate hydrate, as an example. It will be understood in light of the present disclosure that any suitable niobium compounds may be used, and the disclosure is not limited in this regard. It will be further understood in light of the present disclosure that suitable niobium compounds may vary depending on factors, including those described herein.

The sol-gel suspension may include any suitable chelating agent. Suitable chelating agents may be selected, for example, based on desired morphology after synthesis, such as desired particle size or desired packing. Suitable chelating agents may include EDTA (ethylenediaminetetraacetic acid), citric acid, or glycol, as a few examples. It will be understood in light of the present disclosure that any suitable chelating agents may be used and the disclosure is not limited in this regard. It will be further understood in light of the present disclosure that suitable chelating agents may vary depending on factors, including those described herein.

In one or more embodiments, the method 500 may include gelating 520 the sol-gel suspension to form a wet gel. The gelating 520 may include drying the sol-gel suspension. For example, the gelating 520 may occur in a vacuum or a partial vacuum. The gelating 520 may occur at any suitable ambient temperature and may include ramping the ambient temperature. Suitable gelating ambient temperatures may be selected based on desired morphology after synthesis, for example, such as desired particle size or desired packing. Suitable gelating ambient temperatures may include between 50° C. and 250° C. or between 60° C. and 200° C., for example. In one embodiment, gelating 520 may occur by ramping the ambient temperature from approximately 50° C. to approximately 200° C. under vacuum. For further examples, suitable gelating ambient temperatures may include 50° C. or greater, 60° C. or greater, 80° C. or greater, 100° C. or greater, 110° C. or greater, 130° C. or greater, 150° C. or greater, 170° C. or greater, 200° C. or greater, or 220° C. or greater, and/or 250° C. or less, 220° C. or less, 200° C. or less, 170° C. or less, 150° C. or less, 130° C. or less, 110° C. or less, 100° C. or less, 80° C. or less, or 60° C. or less. It will be understood in light of the present disclosure that any suitable gelating ambient temperatures may be used and the disclosure is not limited in this regard. It will be further understood in light of the present disclosure that suitable gelating ambient temperatures may vary depending on factors, including those described herein.

In at least one embodiment, the method 500 may include off-gassing 530 the wet gel to form a dry gel. Without wishing to be bound by theory, off-gassing 530 may be useful to remove byproducts formed in the gelation process, such as nitrates and citrate byproducts.

The off-gassing 530 may occur at any suitable ambient temperature. In some embodiments, the off-gassing 530 may occur while ramping the ambient temperature. Suitable off-gassing ambient temperatures may be selected based on desired off-gassing duration or characteristics of byproducts to be removed (e.g., decomposition temperatures, boiling temperatures, etc.), as two examples. Suitable off-gassing ambient temperatures may include, for example, between 300° C. and 600° C. In one embodiment, the off-gassing 530 may occur at an ambient temperature of approximately 400° C. For further examples, suitable off-gassing ambient temperatures may include 250° C. or greater, 300° C. or greater, 350° C. or greater, 400° C. or greater, 450° C. or greater, 500° C. or greater, 550° C. or greater, or 600° C. or greater, and/or 600° C. or less, 550° C. or less, 500° C. or less, 450° C. or less, 400° C. or less, 350° C. or less, or 300° C. or less. It will be understood in light of the present disclosure that any suitable off-gassing ambient temperatures may be used, and the disclosure is not limited in this regard. It will be further understood in light of the present disclosure that suitable off-gassing ambient temperatures may vary depending on factors, including those described herein.

The off-gassing 530 may occur for any suitable duration, which may be defined, for example, as a time period during which the sample is in a target off-gassing ambient temperature. Suitable off-gassing durations may be selected based on off-gassing temperature or characteristics of byproducts to be removed, as two examples. Suitable off-gassing durations may include, for example, between 2 hours and 10 hours or between 4 hours and 8 hours. In one embodiment, the off-gassing 530 may occur for a duration of approximately 6 hours. For further examples, suitable off-gassing durations may include 2 hours or greater, 3 hours or greater, 4 hours or greater, 5 hours or greater, 6 hours or greater, 8 hours or greater, 9 hours or greater, or 10 hours or greater, and/or 10 hours or less, 9 hours or less, 8 hours or less, 7 hours or less, 6 hours or less, 5 hours or less, or 4 hours or less. It will be understood in light of the present disclosure that any suitable off-gassing duration may be used, and the disclosure is not limited in this regard. It will be further understood in light of the present disclosure that suitable off-gassing durations may vary depending on factors, including those described herein.

In one or more embodiments, the method 500 may include crushing 540 the dry gel.

In some embodiments, the method 500 may include calcinating 550 the dry gel to form the negative electrode active material. The sol-gel calcinating 550 may occur at any suitable ambient temperature. Suitable sol-gel calcinating ambient temperatures may be selected based on desired crystal structure, for example. In some embodiments, the sol-gel calcinating 550 may occur while ramping the ambient temperature, such as ramping the ambient temperature at a heating rate of 10° C. per minute and/or ramping the ambient temperature at a cooling rate of 5° C. per minute, for example. Suitable sol-gel calcinating ambient temperatures may include, for example, between 800° C. and 1,500° C. In one embodiment, the sol-gel calcinating 550 may occur at an ambient temperature of approximately 1,000° C. For further examples, suitable sol-gel calcinating ambient temperatures may include 700° C. or greater, 800° C. or greater, 900° C. or greater, 1,000° C. or greater, 1,200° C. or greater, 1,300° C. or greater, 1,400° C. or greater, or 1,500° C. or greater, and/or 1,500° C. or less, 1,400° C. or less, 1,300° C. or less, 1,200° C. or less, 1,000° C. or less, 900° C. or less, or 800° C. or less. It will be understood in light of the present disclosure that any suitable sol-gel calcinating ambient temperatures may be used and the disclosure is not limited in this regard. It will be further understood in light of the present disclosure that suitable sol-gel calcinating ambient temperatures may vary depending on factors, including those described herein.

The sol-gel calcinating 550 may occur for any suitable duration, which may be defined as a time period during which the sample is in a target sol-gel calcinating ambient temperature, for example. Suitable sol-gel calcinating durations may be selected based on sol-gel calcinating ambient temperature, desired degree of crystallinity, or desired crystallite size, for example. Suitable sol-gel calcinating durations may include, for example, between 1 hour and 6 hours or between 2 hours and 5 hours. In one embodiment, the sol-gel calcinating 550 may occur for a duration of approximately 3 hours. For further examples, suitable sol-gel calcinating durations may include 1 hour or greater, 2 hours or greater, 3 hours or greater, 4 hours or greater, 5 hours or greater, or 6 hours or greater, and/or 7 hours or less, 6 hours or less, 5 hours or less, 4 hours or less, 3 hours or less, 2 hours or less, or 1 hour or less. It will be understood in light of the present disclosure that any suitable sol-gel calcinating durations may be used and the disclosure is not limited in this regard. It will be further understood in light of the present disclosure that suitable sol-gel calcinating durations may vary depending on factors, including those described herein.

Reference will now be made in greater detail to various embodiments of doped titanium niobium oxide negative electrode materials, which may be used in illustrative electrochemical cells described herein, and may furthermore be used in lithium-ion batteries or cells not constructed according to the illustrative embodiments and preferred ranges described herein. Doped titanium niobium oxide negative electrode materials are further described in U.S. Provisional Patent Application Ser. No. 63/564,723 (titled DOPED TITANIUM NIOBIUM OXIDE ANODE MATERIAL), which is incorporated herein by reference in its entirety.

As described herein, negative electrode active materials are provided that include titanium niobium oxide (TNO, represented by the formula TiNb₂O₇) doped with one or more elements. Further provided herein are negative electrode composites including the negative electrode active materials described herein. The TNO negative electrode active materials and/or composites can be included in electrochemical cells such as lithium ion batteries. Batteries that include the doped TNO negative electrode active material may have electrochemical performance properties desirable for medical devices, such as implantable medical devices. For example, batteries that include the doped TNO negative electrode active material described herein may achieve a large energy storage and the ability to charge and/or discharge in short time frames at physiologically relevant temperatures (e.g., 37° C.). Such batteries having large energy storage per unit of material may be able to be miniaturized which may allow for inclusion in implantable medical devices and/or the miniaturization of medical devices that include batteries.

TNO has a relatively high theoretical specific capacity of 387.6 milliampere hours per gram (mAh g⁻¹ or mAh/g). The theoretical specific capacity of TNO is based on three oxidation-reduction (redox) couples Nb5+/Nb4+, Nb4+/Nb3+, and Ti4+/Ti3+. Despite a high theoretical capacity, a more modest initial discharge capacity of around 270 mAh g⁻¹ is typically achieved in TNO negative electrodes constructed outside the embodiments and preferred ranges described herein. Modification to the structure and/or the composition of TNO may allow for the realization of specific capacities closer to the theoretical capacity of TNO.

The TNO negative electrode active materials of the present disclosure include doped TNO. The doped TNO negative electrode active materials include a metal portion and an oxygen portion. The metal portion includes a titanium (Ti) fraction, a niobium (Nb) fraction, and a doped fraction. The titanium fraction consists of titanium. The niobium fraction consists of niobium. The doped metal fraction consists of one or more elemental dopants.

The doped fraction includes one or more elements sometimes referred to as a dopant or dopants. A dopant may or may not be electrochemically active. An elemental dopant may be a metal or phosphorus. The doped fraction may include phosphorus, an alkali metal (group 1 elements excluding hydrogen), an alkaline metal (group 2 elements), a lanthanide (elements having an atomic number of 57 to 70), a transition metal (elements having an atomic number of 21 to 30, 39 to 48, 71 to 80, or 103 to 112), a post-transition metal (Al, Ga, In, Sn, Tl, Pb, and Bi), a metalloid (B, Si, Ge, As, Sb, Te, Po), or any combination thereof. In some embodiments, the doped metal fraction includes an alkali metal, and the alkali metal is sodium (Na), potassium (K), rubidium (Rb), cesium (Cs), or any combination thereof. In some embodiments, the doped fraction includes one or more alkaline metal, and the one or more alkaline metals include magnesium (Mg), calcium (Ca), strontium (Sr), or barium (Ba). In some embodiments, the doped fraction includes one or more lanthanides, and the one or more lanthanides include lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), Ytterbium (Yb) or any combination thereof. In some embodiments, the doped fraction includes one or more transition metals and the one or more transition metals include scandium (Sc), vanadium (V), chromium (Cr), manganese (Mn), iron (Fe), cobalt (Co), nickel (Ni), copper (Cu), zinc (Zn), yttrium (Y), zirconium (Zr), molybdenum (Mo), rhodium (Rh), palladium (Pd), silver (Ag), cadmium (Cd), hafnium (Hf), tungsten (W), rhenium (Re), iridium (Ir), platinum (Pt), gold (Au), or any combination thereof. In some embodiments, the doped fraction includes one or more metalloids, and the one or more metalloids include boron (B), silicon (Si), germanium (Ge), tellurium (Te), or any combination thereof. In some embodiments, the doped fraction includes one or more post-transition metals, and the one or more post-transition metals include aluminum (Al), gallium (Ga), indium (In), tin (Sn), lead (Pb), or any combination thereof.

In some embodiments, the doped fraction includes Re, Ce, Pr, Nd, Pm, Sm, Eu, Gd, Tb, Dy, Ho, Er, Tm, Yt, or any combination thereof. In some embodiments, the doped fraction includes Ce, Tb, Dy, Nd, Re or any combination thereof. In some embodiments, the doped fraction includes Ce. In some embodiments, the doped fraction includes Dy. In some embodiments, the doped fraction includes Nd. In some embodiments, the doped fraction includes Re. In some embodiments, the doped fraction includes Tb.

In some embodiments, the doped fraction includes two or more elements (two or more dopants). In some embodiments, the doped fraction includes Re. In some embodiments, the doped fraction includes Re and a lanthanide.

The metal fractions (e.g., the Ti fraction and the Nb fraction), the doped fraction, and the oxygen portion may be described as the atomic proportions of each of the elements in the doped TNO negative electrode active material relative to one another. The amount of each element may be expressed, for example, as an atomic percent (atm.-%). The oxygen content of the negative electroactive material depends at least in part on the oxidation state of each of the Nb, Ti, and/or the dopant in the compound.

The doped fraction may be present in the metal portion of the negative electrode active material in any suitable amount. In some embodiments, the metal portion of the negative electrode active material includes 10 atm.-% or less, 5 atm.-% or less, 4 atm.-% or less, 3 atm.-% or less, 2 atm.-% or less, 1.5 atm.-% or less, 1 atm.-% or less, 0.5 atm.-% or less, 0.25 atm.-% or less, or 0.1 atm.-% or less of the doped fraction. In some embodiments, the metal portion of the negative electrode active material includes 0.01 atm.-% or greater, 0.1 atm.-% or greater, 0.25 atm.-% or greater, 0.5 atm.-% or greater, 0.75 atm.-% or greater 1 atm.-% or greater, 1.5 atm.-% or greater, 2 atm.-% or greater, 3 atm.-% or greater, 4 atm.-% or greater, or 5 atm.-% or greater of the doped fraction. In some embodiments, the metal potion of the negative electrode active material includes 0.01 atm.-% to 10 atm.-%, 0.1 atm.-% to 5 atm.-%, 0.25 atm.-% to 5 atm.-%, 0.5 atm.-% to 5 atm.-%, 0.75 atm.-% to 5 atm.-%, 1 atm.-% to 5 atm.-%, 2 atm.-% to 5 atm.-%, 0.01 atm.-% to 2 atm.-%, 0.1 atm.-% to 2 atm.-%, 0.25 atm.-% to 2 atm.-%, 0.5 atm.-% to 2 atm.-%, 0.75 atm.-% to 2 atm.-%, 1 atm.-% to 2 atm.-%, 0.01 atm.-% to 1 atm.-%, 0.1 amt-% to 1 atm.-%, 0.25 atm.-% to 1 atm.-%, 0.5 atm.-% to 1 atm.-%, 0.75 atm.-% to 1 atm.-%, 1 atm.-% to 2 atm.-%, 2 atm.-% to 10 atm.-%, 3 atm.-% to 10 atm.-%, 4 atm.-% to 10 atm.-%, 5 atm.-% to 10 atm.-%, 2 atm.-% to 5 atm.-%, or 2 atm.-% to 4 atm.-% of the doped fraction. In some embodiments, the metal portion of the negative electrode active material includes 5 atm-% or less of the doped fraction (e.g., 0.01 atm-% to 5 atm-%). In some embodiments, the metal portion of the negative electrode active material includes 2 atm.-% or less (e.g., 0.01 atm-% to 2 atm-%) of the doped fraction. In some embodiments, the metal portion of the negative electrode active material includes 1 atm.-% or less (e.g., 0.01 atm-% to 2 atm-%) of the doped fraction. In some embodiments, the metal portion of the negative electrode active material includes between 0.1 atom-% and 5 atom-% or between 0.01 atom-% and 10 atom-%.

The combination of the titanium fraction, the niobium fraction, and the doped fraction can be referred to as the TiNbM fraction where M is the dopant. The metal portion of the negative electrode active material may include 95 wt-% or greater, 96 wt-% or greater, 97 wt-% or greater, 98 wt-% or greater, 99 wt-% or greater, or 99.5 wt-% or greater TiNbM fraction. In some embodiments, the negative electrode active material consists of the TiNbM fraction. In some embodiments, the negative electrode active material consists substantially of the TiNbM fraction; that is, the negative electrode active material includes 99 wt-% or greater of the TiNbM fraction.

The combination of the metal portion and the oxygen fraction of the negative electrode active material can be referred to as the doped titanium niobium oxide (doped TNO) fraction. The negative electrode active material may include 95 wt-% or greater, 96 wt-% or greater, 97 wt-% or greater, 98 wt-% or greater, 99 wt-% or greater, or 99.5 wt-% or greater doped TNO fraction. In some embodiments, the negative electrode active material consists of the doped TNO fraction. In some embodiments, the negative electrode active material consists substantially of the doped TNO fraction; that is, the negative electrode active material includes 99 wt-% or greater of the doped TNO fraction.

In some embodiments the doped fraction includes a first dopant fraction and a second dopant fraction, each of the first dopant fraction and the second dopant fraction including an elemental dopant (i.e., a first dopant and a second dopant). The amount of each of the first dopant fraction and the second dopant fraction in the metal portion of the negative electrode active material may be any amount such that the doped fraction is 10 atm-% or less of the metal portion. In some embodiments, the first dopant fraction and the second dopant fraction may each independently be present in the metal portion of the negative electrode active material in an amount of 5 atm.-% or less, 4 atm.-% or less, 3 atm.-% or less, or less, 2 atm.-% or less, 1.5 atm.-% or less, 1 atm.-% or less, 0.5 atm.-% or less, 0.25 atm.-% or less, or 0.1 atm.-% or less. In some embodiments, the first dopant fraction and the second dopant fraction may each independently be present in the metal portion in an amount of 0.01 atm.-% or greater, 0.1 atm.-% or greater, 0.25 atm.-% or greater, 0.5 atm.-% or greater, 0.75 atm.-% or greater 1 atm.-% or greater, 1.5 atm.-% or greater, 2 atm.-% or greater, 3 atm.-% or greater, or 4 atm.-% or greater. In some embodiments, the first dopant fraction and the second dopant fraction may each independently be present in the metal portion in an amount of 0.1 atm.-% to 5 atm.-%, 0.25 atm.-% to 5 atm.-%, 0.5 atm.-% to 5 atm.-%, 0.75 atm.-% to 5 atm.-%, 1 atm.-% to 5 atm.-%, 2 atm.-% to 5 atm.-%, 0.01 atm.-% to 2 atm.-%, 0.1 atm.-% to 2 atm.-%, 0.25 atm.-% to 2 atm.-%, 0.5 atm.-% to 2 atm.-%, 0.75 atm.-% to 2 atm.-%, 1 atm.-% to 2 atm.-%, 0.01 atm.-% to 1 atm.-%, 0.1 amt-% to 1 atm.-%, 0.25 atm.-% to 1 atm.-%, 0.5 atm.-% to 1 atm.-%, 0.75 atm.-% to 1 atm.-%, 1 atm.-% to 2 atm.-%, 2 atm.-% to 5 atm.-%, or 2 atm.-% to 4 atm.-%.

The negative electrode active material may have any suitable crystal morphology. Suitable crystal morphologies may include any suitable average crystal size, or particle size. Suitable crystal sizes may be selected based on desired lifecycle of the electrochemical cell, as an example. Without wishing to be bound by theory, larger crystals, or particles, may be more susceptible to fracture during charge and discharge cycles of an electrochemical cell, which may lead to poor extended cycling. In other words, smaller crystals, or particles, such as approximately 500 nm or smaller, for example, may be generally desirable in order to avoid or reduce cracking, or fracture, of the particles. For further examples, suitable average crystal sizes may be selected based on material compatibility, desired power density, and/or desired specific capacity. Without wishing to be bound by theory, larger particles generally afford greater specific capacity, and smaller particles generally afford greater power density. Suitable average crystal lengths may include 0.1 um to 5 um or between 0.3 um to 1 um. Suitable average crystal lengths may include 7 um or less, 5 um or less, 4 um or less, 3 um or less, 2 um or less, 1 um or less, 0.8 um or less, 0.5 um or less, 0.3 um or less, or 0.1 um or less, and/or 0.05 um or greater, 0.1 um or greater, 0.3 um or greater, 0.5 um or greater, 0.8 um or greater, 1 um or greater, 2 um or greater, 3 um or greater, 4 um or greater, or 5 um or greater.

The negative electrode active material may be a single phase material or a multiphase material. The phase of the material may be determined using powder X-ray diffraction (PXRD), such as the PXRD Test Method (see the Test Method section of the present disclosure). A single phase material has one distinct compound visualized as a single crystal structure in a PXRD trace. A multiphase material includes two or more distinct compounds visualized as two or more crystal structures in a PXRD trace.

Compared to undoped TNO, it is thought that titanium and/or niobium atoms are substituted with dopant atoms to form the primary phase. Stated differently, the dopant atoms are integrated into the TNO lattice. In embodiments where all of the dopant atoms are integrated into the TNO lattice, the material is single phase. In embodiments, where some dopant atoms are not integrated into the TNO lattice, the material is multiphase.

In some embodiments, the electrode active material includes a primary phase and/or secondary phase. The primary phase may include compounds that include titanium, niobium, oxygen, and the dopant. In embodiments, where a secondary phase exists, the secondary phase may include compounds lacking one or more of the elements present in the compound of the primary phase. For example, the secondary phase may include dopant-oxides. Without wishing to be bound by theory, it is thought that existence of a secondary phase may be due in part to the concentration of the dopant in the material. For example, it is thought that at a certain concentration of dopant atoms, the TNO lattice reaches a saturation point in which no more dopant atoms can be integrated. The non-integrated dopant atoms become part of the secondary phase. The dopant saturation concentration may vary depending on the identity of the dopant.

The negative electrode active material may be of Formula I:

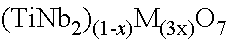

where M is a dopant and x is greater than 0 and less than 1. The O₇ portion of the formula indicates that the average number of oxygen atoms is expected to be 7; however, the actual average number of oxygen atoms is dependent on the oxidation state of Ti, Nb, and M. As such, the atomic-% of oxygen may vary. The dopant may be any elemental dopant as described herein.

The value of x in Formula I may be 0.01 or greater, 0.02 or greater, 0.03 or greater, 0.04 or greater, 0.05 or greater, 0.06 or greater, 0.07 or greater, 0.08 or greater, 0.09 or greater, 0.1 or greater, 0.15 or greater, 0.2 or greater, or 0.25 or greater. The value of x in Formula I may be 0.3 or less, 0.25 or less, 0.2 or less, 0.15 or less, 0.1 or less, 0.09 or less, 0.08 or less, 0.07 or less, 0.06 or less, 0.05 or less, 0.04 or less, 0.03 or less, or 0.02 or less. In some embodiments the value of x in Formula I is 0.01 to 0.3, 0.01 to 0.25, 0.01 to 0.15, 0.01 to 0.1, 0.01 to 0.08, 0.02 to 0.08, 0.02 to 0.07, 0.02 to 0.06, 0.05 to 0.1, or 0.05 to 0.08.

In some embodiments, the negative electrode active material of Formula I may be a single phase material according to the PXRD Test Method. In some embodiments, the negative electrode active material of Formula I may be a multiphase material according to the PXRD Test Method.

The doped TNO negative electrode active materials of the present disclosure may be included in a negative electrode composite. In addition to the negative electrode active material, a negative electrode composite may include a binder, electrically conductive additive, or both.

In some embodiments, the negative electrode composite may include a binder. The binder may be or include any suitable material or combination of materials. Suitable binder materials may be selected based on material compatibility with the negative electrode active material and/or material compatibility with other components of the electrochemical cell in which it may be used, density, porosity, tortuosity, elastic modulus, or degree of swelling in electrolyte, as examples. Suitable binder materials may include polyvinylidene fluoride (PVFD), polytetrafluoroethylene (PTFE), styrene butadiene rubber (SBR), carboxy methyl cellulose (CMC), polyimides, or any combination thereof.

In some embodiments, the doped TNO negative electrode composite may include an electrically conductive additive. The electrically conductive additive may be or include any suitable material or combination of materials. Suitable electrically conductive additive materials may be selected based on material compatibility with the negative electrode active material and/or material compatibility with other components of the electrochemical cell in which it may be used, conductivity, particle surface area, density, porosity, tortuosity electronic conductivity, short range electronic conductivity, or long-range electronic conductivity, as examples. Suitable electrically conductive additive materials may include carbon such as carbon black, carbon nanotubes, single wall carbon nanotubes, multiwall carbon nanotubes, graphite, or graphene, or any combination thereof.

The doped TNO negative electrode composite may have any suitable porosity. Porosity may be determined, or measured, using any suitable method or technique, such as Mercury Intrusion Porosimetry or Electromechanical Impedance Spectroscopy. Suitable negative electrode composite porosities may be selected based on desired energy density or material properties such as particle size, particle shape, particle surface area, and/or desired power density. Without wishing to be bound by theory, a higher porosity is thought to generally afford higher power. In some embodiments, the negative electrode composite may have a porosity of 10% to 70% or 15% to 50%. In some embodiments, the negative electrode composite may have a porosity of 80% or less, 70% or less, 60% or less, 50% or less, 40% or less, 30% or less, or 20% or less. In some embodiments, the negative electrode composite may have a porosity of 5% or greater, 10% or greater, 15% or greater, 20% or greater, 30% or greater, 40% or greater, 50% or greater, 60% or greater, or 70% or greater.

The doped TNO negative electrode composite may have any suitable surface area. In some embodiments, the negative electrode composite may have a surface area of 0.5 square centimeters (cm²) to 300 cm². In some embodiments, the negative electrode composite may have a surface area of 0.5 cm² or greater, 1 cm² or greater, 5 cm² or greater, 10 cm² or greater, 25 cm² or greater, 50 cm² or greater, 100 cm² or greater, 150 cm² or greater, 200 cm² or greater, 250 cm² or greater, or 300 cm² or greater, and/or 300 cm² or less, 250 cm² or less, 200 cm² or less, 150 cm² or less, 100 cm² or less, 50 cm² or less, 25 cm² or less, 10 cm² or less, 5 cm² or less, 1 cm² or less, or 0.5 cm² or less.

The doped TNO active materials and compositions described herein—and negative electrodes or negative electrode composites including doped TNO compositions described herein—may be used in electrochemical cells, such as illustrative electrochemical cells described herein, and may furthermore be used in lithium-ion batteries or cells not constructed according to the illustrative embodiments and preferred ranges described herein. The properties of a negative electrode active material may impact the electrochemical properties of an electrochemical cell. The electrochemical properties of the negative electrode active material can be evaluated when the negative electrode active material is included in a working electrode of a half cell. Half cells include a working electrode and a reference electrode. The working electrode is the limiting electrode and the electrode for which the properties are being evaluated. The reference electrode completes the electrochemical cell but does not limit the rate of the electrochemical reaction. As such, use of the negative electrode active material in the working electrode in a half cell can be used to measure or derive various electrochemical properties of the negative electrode active material. Although the negative electrode active material, composite including the same, or electrode including the same may be included in and function as an anode during the discharge of an electrochemical cell, when evaluated in the half cell, the electrode that includes the negative electrode active material functions as the cathode. Examples of electrochemical performance properties include specific capacity, average discharge voltage, rate retention, capacity retention, irreversible capacity, and diffusion coefficient or diffusivity. Unless otherwise stated, the electrochemical performance properties (e.g., specific capacity, average discharge voltage, rate retention, capacity retention, irreversible capacity, and diffusion coefficient or diffusivity) are described when the negative electrode active material, composite including the same, or electrode is included as in the working electrode of a half cell.

As described herein, electrochemical properties of electrochemical cells may depend at least in part on the working temperature of the electrochemical cell. The working temperature is the ambient temperature that the electrochemical cell is exposed to during cycling. Examples of working temperatures include room temperature and physiologically relevant temperatures. Throughout this disclosure, room temperature refers to a temperature that is 20° C. to 25° C. Physiologically relevant temperatures include internal temperatures of living organisms, such as humans (e.g., 37° C.). The working temperature may or may not be the same temperature as the electrochemical cell itself. For implantable medical devices, it may be desirable to have an electrochemical cell that has strong electrochemical performance properties at a physiologically relevant working temperature such as 37° C.

Specific capacity, or discharge capacity, is the amount of energy in an electroactive material compared to the mass of the electroactive material and may be expressed, for example, as milliamperes-hours per gram of electroactive material (mAh/g or mAh g⁴). Specific capacity is dependent at least in part on the electroactive material (in this case the doped TNO negative electrode active material). Specific capacity of the doped TNO negative electrode active material, composite including the same, or electrode containing the same can be measured according to the Specific Capacity Test Method (see the Test Methods described herein). In some embodiments, doped TNO negative electroactive material of the present disclosure may have improved specific capacity at a working temperature of room temperature and/or 37° C. compared to undoped TNO negative electroactive material. In some embodiments, the negative electroactive material composite including the same, or electrode containing the same has a specific capacity at a room temperature working temperature and/or a 37° C. working temperature of 250 mAh/g or greater, 275 mAh/g or greater, 300 mAh/g or greater, 310 mAh/g or greater, 320 mAh/g or greater, 330 mAh/g or greater, 340 mAh/g or greater, 350 mAh/g or greater, 360 mAh/g or greater, or 370 mAh/g or greater as measured according to the Specific Capacity Test Method. In some embodiments, the negative electroactive material, composite including the same, or electrode containing the same has a specific capacity at a room temperature working temperature and/or a 37° C. working temperature of 380 mAh/g or less, 370 mAh/g or less, 360 mAh/g or less, 350 mAh/g or less, 340 mAh/g or less, 330 mAh/g or less, 320 mAh/g or less 310 mAh/g or less, 300 mAh/g or less, or 275 mAh/g or less as measured according to the Specific Capacity Test Method.

Average discharge voltage is voltage an electrode operates at (on average) during a charge-discharge cycle. In a half cell, the average discharge voltage is the voltage the working electrode (in this case, the electrode that includes the doped TNO negative electrode active material) compared to the reference electrode. Average discharge voltage may be measured according to the Average Voltage Test Method and be expressed as volts (V) vs the identity of the reference electrode (e.g., 1.5 V vs Li/Li+). In some embodiments, the doped TNO negative electroactive material of the present disclosure may have improved or similar average discharge voltage at a working temperature of room temperature and/or 37° C. compared to the undoped TNO negative electroactive material. In some embodiments, the negative electroactive material, composite including the same, or electrode including the same has an average discharge voltage at a room temperature working temperature and/or a 37° C. working temperature of 1.0 V to 2.5 V vs Li/Li+(counter electrode) or 1.2 V to 2.2 V vs Li/Li+ as measured according to the Average Voltage Test Method. In some embodiments, the negative electroactive material, composite including the same, or electrode including the same may have an average discharge voltage at a room temperature working temperature and/or a 37° C. working temperature of 1.0 V or greater, 1.2 V or greater, 1.4 V or greater, 1.5 V or greater, 1.6 V or greater, 1.7 V or greater, 1.8 V or greater, 1.9 V or greater, 2 V or greater, 2.1 V or greater, or 2.2 V or greater, and/or 2.5 V or less, 2.2. V or less, 2.2 V or less, 2.1 V or less, 2 V or less, 1.9 V or less, 1.8 V or less, 1.7 V or less, 1.6 V or less, 1.5 V or less, 1.4 V or less, or 1.2 V or less vs Li/Li+ Average Voltage Test Method.

In some embodiments, it may be beneficial for a doped TNO negative electrode active material, a composite including the same, or an electrode including the same to have a high specific capacity and a low average discharge voltage to maximize the energy at a low voltage. In some embodiments, the negative electrode active, composite including the same, or electrode including the same material has a capacity of 300 mAh/g or greater, 310 mAh/g or greater, 320 mAh/g or greater, 330 mAh/g or greater, 340 mAh/g or greater, 350 mAh/g or greater, 360 mAh/g or greater as measured according to the Specific Capacity Test Method and has an average discharge voltage of 2.2V or less, 2.1 V or less, 2.1 V or greater, 2 V or less, 1.9 V or less, 1.8 V or less, 1.7 V or less, 1.6 V or less, 1.5 V or less, 1.4 V or less, or 1.2 V or less vs Li/Li+ Average Voltage Test Method.

Electrochemical cells that include the doped TNO negative electrode active material, a composite including the same, or an electrode including the same may display high cycling stability. For example, such electrochemical cells may have a high capacity retention and/or a high discharge rate retention. High capacity retentions and/or high rate retentions may allow the electrochemical cell to meet the capacity and power needs of a medical device, such as an implantable medical device, over the course of many charge-discharge cycles.

Capacity retention measures an electrode's specific capacity over the course of multiple charge-discharge cycles as a percentage of the electrode's specific capacity in the electrode's first cycle. Capacity retention can be measured according to the Capacity Retention Test Method (see the Test Methods described herein). In some embodiments, the doped TNO negative electroactive material of the present disclosure may have improved or similar capacity retention at a working temperature of room temperature and/or 37° C. compared to the undoped TNO negative electroactive material. In some embodiments, the negative electroactive material, composite including the same, or electrode including the same may have energy capacity retention after 10 charge-discharge cycles at a working temperature of room temperature and/or a working temperature of 37° C. of 75% to 99.5% or 80% to 99% as measured according to the Capacity Retention Test Method. In some embodiments, the negative electroactive material, composite including the same, or electrode including the same may have energy capacity retentions after 10 charge-discharge cycles at a working temperature of room temperature and/or a working temperature of 75% or greater, 80% or greater, 85% or greater, 90% or greater, 95% or greater, 97% or greater, 98% or greater, or 99% or greater, and/or 99.5% or less, 99% or less, 98% or less, 97% or less, 95% or less, 90% or less, 85% or less, 80% or less, or 75% or less as measured according to the Capacity Retention Test Method. In some embodiments, the negative electroactive material, composite including the same, or electrode including the same has an energy capacity retention of over 100% as measured according to the Capacity Retention Test Method.

Discharge rate retention measures an electrode's discharge rate (that is, the time rate of energy transfer of which the electrode is capable) over the course of multiple charge-discharge cycles as a percentage of the electrode's discharge rate in the electrode's first cycle. Discharge rate retention can be measured according to the Discharge Rate Retention Test Method (see the Test Methods described herein). In some embodiments, the doped TNO negative electroactive material of the present disclosure may have improved or similar discharge rate retention at a working temperature of room temperature and/or 37° C. compared to undoped TNO negative electroactive material. In some embodiments, the negative electroactive material, composite including the same, or electrode including the same may have discharge rate retentions after 10 charge-discharge cycles at a working temperature of room temperature and/or 37° C. of 70% to 99% or 75% to 99% as measured according to the Discharge Rate Retention Test Method. In some embodiments, the negative electroactive material, composite including the same, or electrode including the same may have discharge rate retentions after 10 charge-discharge cycles at a working temperature of room temperature and/or 37° C. of 70% or greater, 75% or greater, 80% or greater, 85% or greater, 90% or greater, 95% or greater, or 97% or greater, and/or 99% or less, 97% or less, 95% or less, 90% or less, 85% or less, 80% or less, or 75% or less, or 70% or less as measured according to the Discharge Rate Retention Test Method.

In lithium-ion electrochemical cells, redox reactions occur when the lithium ions diffuse into an electrode active material (e.g., a doped TNO negative electrode active material). The ability of lithium ions to diffuse into and out of an electrode active material may impact one or more electrochemical performance properties of an electrochemical cell. The movement of lithium ions into and out of an electrode active material can be described as electrochemical cell diffusivity (D). Generally, a higher diffusivity may result in improved electrochemical performance properties. Diffusivity can be measured, for example, according to the Diffusion Coefficient Test Method (see the Test Methods described herein).

In some embodiments, a the doped TNO negative electroactive material of the present disclosure may have improved or similar diffusivity at a working temperature of room temperature and/or 37° C. compared to the same undoped TNO negative electroactive material. In some embodiments, the negative electroactive material, composite including the same, or electrode including the same may have a diffusivity at a working temperature of room temperature and/or 37° C. of 1×10⁻⁹ square centimeters per second (cm²/s) to 5×10⁻⁷ cm²/s or 1×10⁻⁹ cm²/s to 2×10⁻⁷ cm²/s as measured according to the Diffusivity Test Method. In some embodiments, the negative electroactive material, composite including the same, or electrode including the same may have a diffusivity at a working temperature of room temperature and/or 37° C. of 1×10⁻⁹ or greater, 2×10⁻⁹ or greater, 3×10⁻⁹ or greater, 5×10⁻⁹ or greater, 7×10⁻⁹ or greater, 1×10⁻⁸ cm²/s or greater, 2×10⁻⁸ cm²/s or greater, 5×10⁻⁸ cm²/s or greater, 7×10⁻⁸ cm²/s or greater, 9×10⁻⁸ cm²/s or greater, 1×10⁻⁷ cm²/s or greater, 3×10⁻⁷ cm²/s or greater, or 5×10⁻⁷ cm²/s or greater, and/or 5×10⁻⁷ cm²/s or less, 3×10⁻⁷ cm²/s or less, 1×10⁻⁷ cm²/s or less, 9×10⁻⁸ cm²/s or less, 7×10⁻⁸ or less, 5×10⁻⁸ cm²/s or less, 2×10⁻⁸ cm²/s or less, 1×10⁻⁸ cm²/s or less, 7×10⁻⁹ or less, 5×10⁻⁹ or less, 3×10⁻⁹ or less, or 2×10⁻⁹ or less as measured according to the Diffusivity Test Method. In some embodiments, the negative electroactive material, composite including the same, or electrode including the same may have a diffusivity at a working temperature of room temperature and/or 37° C. of 1×10⁻⁹ to 5×10⁻⁸ cm²/s, 1×10⁻⁹ to 2×10⁻⁸ cm²/s, or 1×10⁻⁹ to 1×10⁻⁸ cm²/s as measured according to the Diffusivity Test Method.

Irreversibility capacity is the first charge capacity minus the first discharge capacity of an electrode (e.g., the working electrode) and may be expressed, for example, as a percent of the first charge capacity. Charge capacity is the total amount of energy that can be stored in an electrode when fully charge and discharge capacity is the total amount of energy that has been drawn from the electrode when fully discharged. Irreversibility capacity can be measured according to the Irreversibility Capacity Test Method (see the Test Methods described herein). Generally, a lower irreversible capacity is desired. In some embodiments, the doped TNO negative electroactive material of the present disclosure may have improved or similar average irreversibility capacity at a working temperature of room temperature and/or 37° C. compared to undoped TNO negative electroactive material. In some embodiments, the negative electroactive material, composite including the same, or electrode including the same has an irreversibility capacity at a room temperature working temperature and/or a 37° C. working temperature of 25% or less, 20% or less, 15%, 10% or less, 5% or less, or 2.5% or less as measured according to the Irreversibility Test Method.

Without wishing to be bound by theory, it is thought that the ionic radius of the dopant in the doped-TNO electroactive material may impact one or more of the electrochemical performance properties of an electrochemical cell or electrode that includes the doped-TNO electroactive material. Dopant elements with large ionic radii (e.g., a Shannon radii between 0.9 angstroms and 1.0 angstroms) may result in electrochemical cells having improved specific capacity. It is thought that the presence of the large dopant atoms in the TNO lattice may increase the size of channels within the TNO crystal structure allowing for more facile lithium ion diffusion.

Additionally, without wishing to be bound by theory, it is thought that doped TNO negative electrode active materials having a dopant in an amount sufficient for percolation in the next-nearest neighbor lattice network may be beneficial in improving one or more electrochemical performance properties cells having a TNO based negative electrode active material. Ideal dopants in the next-nearest neighbor lattice regime may disturb a volume of space around them out to their second neighbor in the lattice network. This disturbance may allow for increased size of channels through the entire TNO crystal and increased lithium ion diffusion.

The doped TNO electrode active materials described herein may be formed using any suitable synthesis method. Suitable synthesis methods to form the doped TNO negative electrode active material may include solid-state synthesis, sol-gel synthesis, or hydrothermal synthesis. In the different methods, the doped TNO negative electrode active material is formed from Ti, Nb, and dopant precursor compounds. The precursor compounds include one or more of the elements found in the doped TNO negative electrode active materials described herein.

An illustrative solid-state method of synthesizing the doped TNO negative electrode active material may include mixing oxide precursors of each of each element (e.g., Ti, Nb, and the dopant), such as oxide precursor powders. The mixing may include any suitable mixing procedure. Suitable mixing procedures may include using a mortar and pestle and/or using a ball mill, for just two examples. Any suitable precursors may be used such as oxide powders. Suitable oxide precursors may include, for example, Nb₂O₅ powder, TiO₂ powder, and dopant oxide powder. In some embodiments, the solid-state method of synthesizing the negative electrode active material may optionally include pelletizing the mixed oxide precursors.

In one or more embodiments, the solid-state method may include calcinating the mixed precursors (e.g., pelletized mixed precursors). The solid-state calcinating may occur at any suitable ambient temperature. Suitable solid-state calcinating ambient temperatures may be selected based on desired crystal structure, for example. In some embodiments, the solid-state calcinating may occur while ramping the ambient temperature, such as ramping the ambient temperature at a heating rate of 10° C. per minute and/or ramping the ambient temperature at a cooling rate of 5° C. per minute, for example. Suitable solid-state calcinating ambient temperatures may include, for example, 1,000° C. to 2,500° C. For further examples, suitable solid-state calcinating ambient temperatures may include 900° C. or greater, 1,000° C. or greater, 1,300° C. or greater, 1,500° C. or greater, 1,700° C. or greater, 2,000° C. or greater, 2,200° C. or greater, or 2,500° C. or greater, and/or 2,500° C. or less, 2,200° C. or less, 2,000° C. or less, 1,700° C. or less, 1,500° C. or less, 1,300° C. or less, or 1,000° C. or less.

The solid-state calcinating may occur for any suitable duration, which may be defined as a time period during which the sample is in a target solid-state calcinating ambient temperature, for example. Suitable solid-state calcinating durations may be selected based on solid-state calcinating ambient temperature, desired degree of crystallinity, or desired crystallite size, for example. Suitable solid-state calcinating durations may include, for example, between 10 hours and 10 days or between 12 hours and 5 days. For further examples, suitable solid-state calcinating durations may include 10 hours or greater, 12 hours or greater, 18 hours or greater, 2 days or greater, 5 days or greater, or 10 days or greater, and/or 10 days or less, 5 days or less, 2 days or less, 18 hours or less, or 12 hours or less.

In some embodiments, a sol-gel method may be used to synthesize the doped TNO negative electroactive material of the present disclosure, such as the illustrative sol-gel method 500 shown in FIG. 5.

In some embodiments, the sol-gel synthesis method 500 may include preparing a sol-gel suspension 510. The sol-gel suspension may include a titanium compound, a niobium compound, a dopant compound, and a chelating agent.

The sol-gel suspension may include any suitable titanium compound. Suitable titanium compounds may be selected based on solubility in desired solvent (e.g., water, alcohol, acid, etc.) or counter ions (e.g., nitrates, acetates, etc., which may decompose during synthesis), for example. Suitable titanium compounds may include titanium butoxide or titanium(IV) bis(ammonium lactato)dihydroxide, as an example.

The sol-gel suspension may include any suitable niobium compound. Suitable niobium compounds may be selected based on solubility in desired solvent (e.g., water, alcohol, acid, etc.), or counter ions (e.g., nitrates, acetates, etc., which may decompose during synthesis), for example. Suitable niobium compounds may include ammonium niobate oxalate hydrate, as an example.

The sol-gel suspension may include any suitable dopant compound. Suitable dopant compounds include those described in Jonderian, A.; Peng, R.; Davies, D.; McCalla, E. Benefits and Limitations of 226 Substitutions into Li—La—Ti—O Perovskites. Chemistry of Materials 2023, 35 (16), 6227-6234.

The sol-gel suspension may include any suitable chelating agent. Suitable chelating agents may be selected, for example, based on desired morphology after synthesis, such as desired particle size or desired packing. Suitable chelating agents include, for example, include EDTA (ethylenediaminetetraacetic acid), citric acid, and glycol.

In some embodiments, the sol-gel method 500 may include gelating the sol-gel suspension to form a wet gel (step 520). The gelating step may include drying the sol-gel suspension. For example, gelating may occur in a vacuum or a partial vacuum. Gelating may occur at any suitable temperature and may include ramping the ambient temperature. Suitable gelating temperatures may be selected based on desired morphology after synthesis, for example, such as desired particle size or desired packing. Suitable gelating temperatures may include, for example, 50° C. to 250° C. or 60° C. to 200° C. In some embodiments, gelating may occur by ramping the temperature from approximately 50° C. to approximately 200° C. under vacuum. Additional suitable gelating temperatures may include 50° C. or greater, 60° C. or greater, 80° C. or greater, 100° C. or greater, 110° C. or greater, 130° C. or greater, 150° C. or greater, 170° C. or greater, 200° C. or greater, or 220° C. or greater, and/or 250° C. or less, 220° C. or less, 200° C. or less, 170° C. or less, 150° C. or less, 130° C. or less, 110° C. or less, 100° C. or less, 80° C. or less, or 60° C. or less.

In some embodiments, the sol-gel method 500 may include off-gassing the wet gel to form a dry gel (step 530). Without wishing to be bound by theory, off-gassing may be useful to remove byproducts formed in the gelation process, such as nitrates and citrate byproducts.

Off-gassing may occur at any suitable temperature. In some embodiments, the off-gassing may occur while ramping the temperature. Suitable off-gassing ambient temperatures may be selected based on desired off-gassing duration or characteristics of byproducts to be removed (e.g., decomposition temperatures, boiling temperatures, etc.), as two examples. Suitable off-gassing temperatures may include, for example, 300° C. to 600° C. In some embodiments, the off-gassing may occur at a temperature of approximately 400° C. For further examples, suitable off-gassing temperatures may include 250° C. or greater, 300° C. or greater, 350° C. or greater, 400° C. or greater, 450° C. or greater, 500° C. or greater, 550° C. or greater, or 600° C. or greater, and/or 600° C. or less, 550° C. or less, 500° C. or less, 450° C. or less, 400° C. or less, 350° C. or less, or 300° C. or less.

The off-gassing may occur for any suitable duration, which may be defined, for example, as a time period during which the sample is in a target off-gassing temperature. Suitable off-gassing durations may be selected based on off-gassing temperature or characteristics of byproducts to be removed, as two examples. Suitable off-gassing durations may include, for example, 2 hours to 10 hours or 4 hours to 8 hours. In some embodiments, the off-gassing may occur for a duration of approximately 6 hours. For further examples, suitable off-gassing durations may include 2 hours or greater, 3 hours or greater, 4 hours or greater, 5 hours or greater, 6 hours or greater, 8 hours or greater, 9 hours or greater, or 10 hours or greater, and/or 10 hours or less, 9 hours or less, 8 hours or less, 7 hours or less, 6 hours or less, 5 hours or less, or 4 hours or less.

In some embodiments, the sol-gel method 500 may include crushing the dry gel (step 540).

In some embodiments, the sol-gel method 500 may include calcinating the dry gel to form the negative electrode active material (step 550). The sol-gel calcinating may occur at any suitable temperature. Suitable sol-gel calcinating temperatures may be selected based on desired crystal structure, for example. In some embodiments, the sol-gel calcinating may occur while ramping the temperature, such as ramping the temperature at a heating rate of 10° C. per minute and/or ramping the temperature at a cooling rate of 5° C. per minute, for example. Suitable sol-gel calcinating temperatures may include, for example, between 800° C. and 1,500° C. In some embodiments, the sol-gel calcinating may occur at a temperature of approximately 1,000° C. For further examples, suitable sol-gel calcinating temperatures may include 700° C. or greater, 800° C. or greater, 900° C. or greater, 1,000° C. or greater, 1,200° C. or greater, 1,300° C. or greater, 1,400° C. or greater, or 1,500° C. or greater, and/or 1,500° C. or less, 1,400° C. or less, 1,300° C. or less, 1,200° C. or less, 1,000° C. or less, 900° C. or less, or 800° C. or less.

The sol-gel calcinating may occur for any suitable duration, which may be defined as a time period during which the sample is in a target sol-gel calcinating ambient temperature, for example. Suitable sol-gel calcinating durations may be selected based on sol-gel calcinating temperature, desired degree of crystallinity, or desired crystallite size, for example. Suitable sol-gel calcinating durations may include, for example, 1 hour to 6 hours or 2 hours to 5 hours. In some embodiments, the sol-gel calcinating may occur for a duration of approximately 3 hours. For further examples, suitable sol-gel calcinating durations may include 1 hour or greater, 2 hours or greater, 3 hours or greater, 4 hours or greater, 5 hours or greater, or 6 hours or greater, and/or 7 hours or less, 6 hours or less, 5 hours or less, 4 hours or less, 3 hours or less, 2 hours or less, or 1 hour or less.

### Illustrative Aspects

Aspect A1 is a rechargeable lithium-ion cell comprising:
a negative electrode comprising a negative electrode current collector and a negative electrode active material comprising a niobium-based oxide;
a positive electrode comprising a positive electrode current collector and a positive electrode active material comprising a lithium transition metal oxide;
a separator between the negative electrode and the positive electrode; and an electrolyte material;
wherein the cell has an operating voltage of 1.5 V to 4 V or 1.8 V to 3.4 V.

Aspect A2 is the cell of aspect A1, wherein the niobium-based oxide comprises Nb₂O₅, a Nb—Ti—O oxide, a Nb—W—O oxide, a Nb—Ti—W—O oxide, or a combination of two or more thereof.

Aspect A3 is the cell of any one of aspects A1-A2, wherein the niobium-based oxide is represented by the formula:

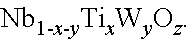

Aspect A4 is the cell of any one of aspects A1-A3, wherein the niobium-based oxide is a pseudoternary composition comprising a metal portion comprising a niobium fraction, a titanium fraction, and a tungsten fraction, and optionally wherein the pseudoternary composition is represented by the formula:

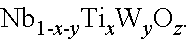

Aspect A5 is the cell of any one of aspects A1-A4, wherein the niobium-based oxide comprises:
a metal portion comprising:
a titanium fraction;
a niobium fraction; and
a doped fraction comprising a dopant, wherein the metal portion comprises 10 atom-% or less, 5 atom-% or less, or between 0.1 atom-% and 5 atom-% of the doped metal fraction; and an oxygen portion.

Aspect A6 is the cell of any one of aspects A1-A5, wherein the niobium-based oxide comprises:
a metal portion comprising:
a titanium fraction;
a niobium fraction; and
a doped metal fraction comprising rhenium, cerium, praseodymium, neodymium, promethium, samarium, europium, gadolinium, dysprosium, holmium, erbium, thulium, ytterbium, or a combination of two or more thereof, and an oxygen portion.

Aspect A7 is the cell of any one of aspects A1-A6, wherein the negative electrode further comprises a carbon coating, a binder, an electrically conductive additive, or a combination of two or more thereof, and optionally wherein the negative electrode active material comprises the carbon coating.

Aspect A8 is the cell of any one of aspects A1-A7, wherein the negative electrode active material has a lithiation potential compared to Li+/Li of between 0.5 V and 3.5 V or between 1.0 V and 3.0 V.

Aspect A9 is the cell of any one of aspects A1-A8, wherein the negative electrode has a negative electrode area specific capacity of between 1 mAh/cm² and 6 mAh/cm².

Aspect A10 is the cell of any one of aspects A1-A9, wherein the lithium transition metal oxide comprises LiCoO₂; LiCoMnO₄; LiCoPO₄; LiNi_{0.5}Mn_{1.5}O₄; a composition represented by the formula LiNiₓMn_{y}Co_{1-x-y}O₂; a composition represented by the formula LiNiₓCo_{y}Al_{1-x-y}O₂; a lithium-rich layered oxide represented by the formula Li₁+xTM₁−xO₂, wherein TM represents a transition metal; or a combination of two or more thereof.

Aspect A11 is the cell of any one of aspects A1-A10, wherein the positive electrode active material has an operating electrode potential compared to Li+/Li of between 2.8 V and 5 V or between 3 V and 4.5 V.

Aspect A12 is the cell of any one of aspects A1-A11, wherein the positive electrode has a positive electrode area specific capacity of between 1 mAh/cm² and 6 mAh/cm².

Aspect A13 is the cell of any one of aspects A1-A12, wherein the negative electrode defines a negative electrode major surface and one or more negative electrode edges defining a perimeter of the negative electrode major surface;
wherein the positive electrode defines a positive electrode major surface and one or more positive electrode edges defining a perimeter of the positive electrode major surface, the one or more positive electrode edges arranged line-to-line with the one or more negative electrode edges.

Aspect A14 is the cell of any one of aspects A1-A13, wherein the positive electrode has a positive electrode specific capacity and a positive electrode capacity fade rate, and the negative electrode has a negative electrode specific capacity and a negative electrode capacity fade rate; and
wherein a N/P capacity ratio of the negative electrode specific capacity to the positive electrode specific capacity is:
less than 1 when the negative electrode capacity fade rate is less than the positive electrode capacity fade rate; and
greater than 1 when the negative electrode capacity fade rate is greater than the positive electrode capacity fade rate.

Aspect A15 is the cell of any one of aspects A1-A14, wherein the positive electrode has a positive electrode specific capacity and a positive electrode capacity fade rate, and the negative electrode has a negative electrode specific capacity and a negative electrode capacity fade rate;
wherein a N/P capacity ratio of the negative electrode specific capacity to the positive electrode specific capacity is less than 1; and
wherein the negative electrode capacity fade rate is less than the positive electrode capacity fade rate.

Aspect A16 is the cell of any one of aspects A1-A15, wherein the positive electrode has a positive electrode specific capacity and a positive electrode capacity fade rate, and the negative electrode has a negative electrode specific capacity and a negative electrode capacity fade rate;
wherein a N/P capacity ratio of the negative electrode specific capacity to the positive electrode specific capacity is greater than 1; and
wherein the negative electrode capacity fade rate is greater than the positive electrode capacity fade rate.

Aspect A17 is the cell of any one of aspects A1-A16, wherein the positive electrode has a positive electrode first-cycle irreversible capacity, and the negative electrode has a negative electrode first-cycle irreversible capacity; and
wherein the negative electrode current collector comprises:
copper when the negative electrode first-cycle irreversible capacity is less than the positive electrode first-cycle irreversible capacity; and
aluminum when the negative electrode first-cycle irreversible capacity is greater than the positive electrode first-cycle irreversible capacity.

Aspect A18 is the cell of any one of aspects A1-A17, wherein the positive electrode has a positive electrode first-cycle irreversible capacity, and the negative electrode has a negative electrode first-cycle irreversible capacity;
wherein the negative electrode active material has an operating voltage of 3 V or greater at 95% delithiation, when the negative electrode first-cycle irreversible capacity is greater than the positive electrode first-cycle irreversible capacity; and
wherein the positive electrode active material has an operating voltage of 3 V or less at 95% lithiation, when the negative electrode first-cycle irreversible capacity is less than the positive electrode first-cycle irreversible capacity.

Aspect A19 is the cell of aspect A18, wherein the negative electrode current collector comprises aluminum, when the negative electrode first-cycle irreversible capacity is greater than the positive electrode first-cycle irreversible capacity; and wherein the negative electrode current collector comprises copper, when the negative electrode first-cycle irreversible capacity is less than the positive electrode first-cycle irreversible capacity.

Aspect A20 is the cell of any one of aspects A1-A19, wherein the positive electrode has a positive electrode first-cycle irreversible capacity, and the negative electrode has a negative electrode first-cycle irreversible capacity;
wherein the negative electrode active material has an operating voltage of 3 V or greater at 95% delithiation; and
wherein the negative electrode first-cycle irreversible capacity is greater than the positive electrode first-cycle irreversible capacity.

Aspect A21 is the cell of any one of aspects A1-A20, wherein the positive electrode has a positive electrode first-cycle irreversible capacity, and the negative electrode has a negative electrode first-cycle irreversible capacity;
wherein the positive electrode active material has an operating voltage of 3 V or less at 95% lithiation; and
wherein the negative electrode first-cycle irreversible capacity is less than the positive electrode first-cycle irreversible capacity.

Aspect A22 is the cell of any one of aspects A1-A21, wherein the positive electrode has a positive electrode first-cycle irreversible capacity, and the negative electrode has a negative electrode first-cycle irreversible capacity;
wherein the negative electrode active material has an operating voltage of 3 V or greater at the end of near full delithiation;
wherein the negative electrode first-cycle irreversible capacity is greater than the positive electrode first-cycle irreversible capacity; and
wherein the negative electrode current collector comprises aluminum.

Aspect A23 is the cell of any one of aspects A1-A22, wherein the positive electrode has a positive electrode first-cycle irreversible capacity, and the negative electrode has a negative electrode first-cycle irreversible capacity;
wherein the negative electrode active material has an operating voltage of 3 V or less at the end of near full delithiation;
wherein the negative electrode first-cycle irreversible capacity is less than the positive electrode first-cycle irreversible capacity; and
wherein the negative electrode current collector comprises copper.

Aspect A24 is an implantable medical device comprising the cell of any one of aspects A1-A24.

Aspect B1 is a negative electrode composite comprising:
a negative electrode active material represented by the formula:

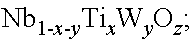

a binder; and
an electrically conductive additive.

Aspect B2 is the negative electrode composite of Aspect B1, wherein the binder comprises polyvinylidene fluoride.

Aspect B3 is the negative electrode composite of any one of aspects B1-B2, wherein the electrically conductive additive comprises one or both of carbon black and carbon nanotubes.

Aspect B4 is the negative electrode composite of any one of aspects B1-B3, wherein a porosity of the negative electrode composite is 50% or less, 15% or greater, or between 15% and 50%.

Aspect B5 is the negative electrode composite of any one of aspects B1-B4, wherein a surface area of the negative electrode composite is 300 cm² or less, 0.5 cm² or greater, or between 0.5 cm² and 300 cm².

Aspect B6 is the negative electrode composite of any one of aspects B1-B5, wherein the negative electrode active material defines a crystal morphology comprising one or more of octahedra centered by Nb and Ti, octahedra centered by Nb and W, octahedra centered by Nb and Ti and W, octahedra centered by W, or tetrahedra centered by W.

Aspect B7 is the negative electrode composite of any one of aspects B1-B6, wherein the negative electrode active material defines a crystal morphology with an average crystal size of 2 um or less, 1 um or less, 0.5 um or less, between 0.1 um and 5 um, or between 0.3 um and 1 um.

Aspect B8 is a negative electrode comprising:
the negative electrode composite of any one of aspects B1-B7; and
a negative electrode current collector electrically coupled to the negative electrode composite.

Aspect B9 is the negative electrode of aspect B8, further comprising a coating layer on a surface of the negative electrode, the coating layer comprising carbon.

Aspect B10 is the negative electrode of any one of aspects B8-B9, wherein a surface area of the negative electrode is 300 cm² or less, 0.5 cm² or greater, or between 0.5 cm² and 300 cm².

Aspect B11 is an electrode composite comprising:
a pseudoternary composition comprising a metal fraction comprising a niobium fraction, a titanium fraction, and a tungsten fraction;
a binder; and
an electrically conductive additive.

Aspect B12 is the electrode composite of aspect B11, wherein the pseudoternary composition is represented by the formula:

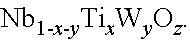

Aspect B13 is the electrode composite of any one of aspects B11-B12, wherein the binder comprises polyvinylidene fluoride.

Aspect B14 is the electrode composite of any one of aspects B11-B13, wherein the electrically conductive additive comprises one or both of carbon black and carbon nanotubes.

Aspect B15 is the electrode composite of any one of aspects B11-B14, wherein a porosity of the electrode composite is 50% or less, 15% or greater, or between 15% and 50%.

Aspect B16 is the electrode composite of any one of aspects B11-B15, wherein a surface area of the electrode composite is 300 cm² or less, 0.5 cm² or greater, or between 0.5 cm² and 300 cm².

Aspect B17 is the electrode composite of any one of aspects B11-B16, wherein the pseudoternary composition defines a crystal morphology comprising one or more of octahedra centered by Nb and Ti, octahedra centered by Nb and W, octahedra centered by Nb and Ti and W, octahedra centered by W, or tetrahedra centered by W.

Aspect B18 is the electrode composite of any one of aspects B11-B17, wherein the pseudoternary composition defines a crystal morphology with an average crystal size of 2 um or less, 1 um or less, 0.5 um or less, between 0.1 um and 5 um, or between 0.3 um and 1 um.

Aspect B19 is the electrode composite of any one of aspects B11-B18, wherein the niobium fraction is 40 at. % or greater, 45 at. % or greater, 50 at. % or greater, 55 at. % or greater, 60 at. % or less, 55 at. % or less, 45 at. % or less, or between 40 at. % and 60 at. %.

Aspect B20 is the electrode composite of any one of aspects B11-B19, wherein the titanium fraction is 50 at. % or less, 45 at. % or less, 40 at. % or less, 35 at. % or less, 30 at. % or greater, 35 at. % or greater, 40 at. % or greater, 45 at. % or greater, or between 30 at. % and 50 at. %.

Aspect B21 is the electrode composite of any one of aspects B11-B20, wherein the tungsten fraction is 25 at. % or less, 20 at. % or less, 15 at. % or less, 10 at. % or less, 5 at. % or greater, 10 at. % or greater, 15 at. % or greater, 20 at. % or greater, between 5 at. % and 25 at. % or between 30 at. % and 75 at. %.

Aspect B22 is an electrode comprising:
the electrode composite of any one of aspects B11-B21; and
a current collector electrically coupled to the electrode composite.

Aspect B23 is the electrode of aspect B22, further comprising a coating layer on a surface of the electrode, the coating layer comprising carbon.

Aspect B24 is the electrode of any one of aspects B22-B23, wherein a surface area of the electrode is 300 cm² or less, 0.5 cm² or greater, or between 0.5 cm² and 300 cm².

Aspect B25 is an electrochemical cell comprising:
a negative electrode comprising:
   a negative electrode active material comprising a pseudoternary composition comprising a metal fraction comprising a niobium fraction, a titanium fraction, and a tungsten fraction;
a positive electrode;
a separator between the negative electrode and the positive electrode; and
an electrolyte.

Aspect B26 is the electrochemical cell of aspect B25, wherein the cell has a specific capacity at ambient room temperature of 200 mAh/g or greater, 250 mAh/g or greater, 300 mAh/g or greater, or 350 mAh/g or greater.

Aspect B27 is the electrochemical cell of any one of aspects B25-B26, wherein the cell has a specific capacity at an ambient temperature of 37° C. of 200 mAh/g or greater, 250 mAh/g or greater, 300 mAh/g or greater, or 350 mAh/g or greater.

Aspect B28 is the electrochemical cell of any one of aspects B25-B27, wherein the cell has an average discharge voltage at ambient room temperature of 2 V or less, 1.8 V or less, 1.7 V or less, 1.6 V or less, or 1.5 V or less.

Aspect B29 is the electrochemical cell of any one of aspects B25-B28, wherein the cell has an average discharge voltage at an ambient temperature of 37° C. of 2 V or less, 1.8 V or less, 1.7 V or less, 1.6 V or less, or 1.5 V or less.

Aspect B30 is the electrochemical cell of any one of aspects B25-B29, wherein the cell has an energy capacity retention after 10 charge-discharge cycles at ambient room temperature of 80% or greater, 90% or greater, 95% or greater, 97% or greater, 98% or greater, or 99% or greater.

Aspect B31 is the electrochemical cell of any one of aspects B25-B30, wherein the cell has an energy capacity retention after 10 charge-discharge cycles at an ambient temperature of 37° C. of 80% or greater, 90% or greater, 95% or greater, 97% or greater, 98% or greater, or 99% or greater.

Aspect B32 is the electrochemical cell of any one of aspects B25-B31, wherein the cell has a discharge rate retention after 10 charge-discharge cycles at room temperature of 75% or greater, 80% or greater, 85% or greater, 90% or greater, or 95% or greater.

Aspect B33 is the electrochemical cell of any one of aspects B25-B32, wherein the cell has a discharge rate retention after 10 charge-discharge cycles at an ambient temperature of 37° C. of 75% or greater, 80% or greater, 85% or greater, 90% or greater, or 95% or greater.

Aspect B34 is the electrochemical cell of any one of aspects B25-B33, wherein the cell has a diffusivity at ambient room temperature of 5×10⁻⁷ cm²/s or less, 1×10⁻⁷ cm²/s or less, or 5×10⁻⁸ cm²/s or less.

Aspect B35 is the electrochemical cell of any one of aspects B25-B34, wherein the cell has a diffusivity at an ambient temperature of 37° C. of 5×10⁻⁷ cm²/s or less, 1×10⁻⁷ cm²/s or less, or 5×10⁻⁸ cm²/s or less.

Aspect B36 is an electrochemical cell comprising:
a negative electrode comprising:
   a negative electrode active material represented by the formula:

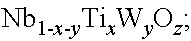

a positive electrode;
a separator between the negative electrode and the positive electrode; and an electrolyte.

Aspect B37 is the electrochemical cell of aspect B36, wherein the cell has a specific capacity at ambient room temperature of 200 mAh/g or greater, 250 mAh/g or greater, 300 mAh/g or greater, or 350 mAh/g or greater.

Aspect B38 is the electrochemical cell of any one of aspects B36-B37, wherein the cell has a specific capacity at an ambient temperature of 37° C. of 200 mAh/g or greater, 250 mAh/g or greater, 300 mAh/g or greater, or 350 mAh/g or greater.

Aspect B39 is the electrochemical cell of any one of aspects B36-B38, wherein the cell has an average discharge voltage at ambient room temperature of 2 V or less, 1.8 V or less, 1.7 V or less, 1.6 V or less, or 1.5 V or less.

Aspect B40 is the electrochemical cell of any one of aspects B36-B39, wherein the cell has an average discharge voltage at an ambient temperature of 37° C. of 2 V or less, 1.8 V or less, 1.7 V or less, 1.6 V or less, or 1.5 V or less.

Aspect B41 is the electrochemical cell of any one of aspects B36-B40, wherein the cell has an energy capacity retention after 10 charge-discharge cycles at ambient room temperature of 80% or greater, 90% or greater, 95% or greater, 97% or greater, 98% or greater, or 99% or greater.

Aspect B42 is the electrochemical cell of any one of aspects B36-B41, wherein the cell has an energy capacity retention after 10 charge-discharge cycles at an ambient temperature of 37° C. of 80% or greater, 90% or greater, 95% or greater, 97% or greater, 98% or greater, or 99% or greater.

Aspect B43 is the electrochemical cell of any one of aspects B36-B42, wherein the cell has a discharge rate retention after 10 charge-discharge cycles at room temperature of 75% or greater, 80% or greater, 85% or greater, 90% or greater, or 95% or greater.

Aspect B44 is the electrochemical cell of any one of aspects B36-B43, wherein the cell has a discharge rate retention after 10 charge-discharge cycles at an ambient temperature of 37° C. of 75% or greater, 80% or greater, 85% or greater, 90% or greater, or 95% or greater.

Aspect B45 is the electrochemical cell of any one of aspects B36-B44, wherein the cell has a diffusivity at ambient room temperature of 5×10⁻⁷ cm²/s or less, 1×10⁻⁷ cm²/s or less, or 5×10⁻⁸ cm²/s or less.

Aspect B46 is the electrochemical cell of any one of aspects B36-B45, wherein the cell has a diffusivity at an ambient temperature of 37° C. of 5×10⁻⁷ cm²/s or less, 1×10⁻⁷ cm²/s or less, or 5×10⁸ cm²/s or less.

Aspect B47 is a method of forming a negative electrode active material, the method comprising:
preparing a sol-gel suspension comprising a tungsten compounds, a titanium compound, a niobium compound, and a chelating agent;
gelating the sol-gel suspension to form a wet gel;
off-gassing the wet gel to form a dry gel; and calcinating the dry gel to form the negative electrode active material.

Aspect B48 is the method of aspect B47, wherein the chelating agent comprises one or more of citric acid, EDTA, or glycol.

Aspect B49 is the method of any one of aspects B47-B48, wherein gelating the sol-gel suspension to form the wet gel occurs in a vacuum or a partial vacuum.

Aspect B50 is the method of any one of aspects B47-B49, wherein gelating the sol-gel suspension to form the wet gel occurs at an ambient temperature between 50° C. and 250° C., 60° C. or greater, 110° C. or greater, 200° C. or greater, 250° C. or less, 120° C. or less, or 80° C. or less.

Aspect B51 is the method of any one of aspects B47-B50, wherein gelating the sol-gel suspension to form the wet gel occurs at an ambient temperature of 60° C. or greater, 110° C. or greater, or 200° C. or greater.

Aspect B52 is the method of any one of aspects B47-B51, wherein gelating the sol-gel suspension to form the wet gel comprises heating the sol-gel suspension at an ambient temperature ramping from 50° C. or greater to 200° C. or greater.

Aspect B53 is the method of any one of aspects B47-B52, wherein off-gassing the wet gel to form the dry gel occurs at an ambient temperature of 300° C. or greater, 400° C. or greater, 450° C. or less, 350° C. or less, or between 300° C. and 600° C.

Aspect B54 is the method of any one of aspects B47-B53, wherein calcinating the dry gel to form the negative electrode active material occurs at an ambient temperature of 800° C. or greater, 1,000° C. or greater, 1,200° C. or less, 900° C. or less, or between 800° C. and 1,500° C.

Aspect B55 is the method of any one of aspects B47-B54, wherein the tungsten compound comprises one or both of tungsten trioxide and ammonium metatungstate hydrate.

Aspect B56 is the method of any one of aspects B47-B55, wherein the titanium compound comprises one or both of titanium butoxide and titanium(IV) bis(ammonium lactato)dihydroxide.

Aspect B57 is the method of any one of aspects B47-B56, wherein the niobium compound comprises ammonium niobate oxalate hydrate.

Aspect C1 is a negative electrode composite comprising:
a negative electrode active material comprising:
   a metal portion comprising:
      a titanium (Ti) fraction;
      a niobium (Nb) fraction; and
      a doped fraction comprising a dopant, wherein the metal portion comprising 5 atom-% of the doped metal fraction; and
   an oxygen portion;
a binder; and
an electrically conductive additive,
wherein when the electrode composite has a specific capacity of 300 mAh/g or greater at 37° C., 20° C. to 25° C., or both as measured according to the Specific Capacity Test Method.

Aspect C2 is the negative electrode composite of aspect C1, wherein the dopant comprises a lanthanide, rhenium, or both.

Aspect C3 is a negative electrode composite comprising:
a negative electrode active material comprising:
   a metal portion comprising:
      a titanium (Ti) fraction;
      a niobium (Nb) fraction; and
      a doped metal fraction comprising rhenium (Re), cerium (Ce), praseodymium (Pr), neodymium (Nd), promethium (Pm), samarium (Sm), europium (Eu), gadolinium (Gd) dysprosium (Dy), holmium (Ho), erbium (Er), Thulium (Tm), Ytterbium (Yt); or any combination thereof; and
   an oxygen portion;
a binder; and
an electrically conductive additive.

Aspect C4 is the negative electrode composite of any one of aspects C1-C3, wherein the metal portion comprises 2.5 atom-% or less, 2 atom-% or less, 1.5 atom-% or less, 1 atom-% or less, or 0.5 atom % or less of the doped fraction.

Aspect C5 is a negative electrode composite comprising:
a negative electrode active material of the formula:

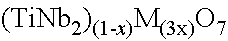

wherein M is a dopant and x is greater than 0 and less than 1;
a binder; and
an electrically conductive additive.

Aspect C6 is the negative electrode composite of aspect C5 wherein x is 0.01 to 0.30.01 to 0.3, 0.01 to 0.25, 0.01 to 0.15, 0.01 to 0.1, 0.01 to 0.08, 0.02 to 0.08, 0.02 to 0.07, 0.02 to 0.06, 0.05 to 0.1, or 0.05 to 0.08.

Aspect C7 is the negative electrode composite of any one of aspects C5-C6, wherein M comprises a transition metal, and wherein the transition meatal is optionally Sc, V, Cr, Mn, Fe, Co, Ni, Cu, Zn, Zr, Nb, Mo, Rh, Pd, Ag, Cd, Hf, W, Re, Ir, Pt, or Au.

Aspect C8 is the negative electrode composite of any one of aspects C5-C7, wherein M comprises an alkali metal or an alkaline metal and wherein the alkali metal or alkaline meta is optionally Na, Mg, K, Ca, Rb, Sr, Cs, or Ba.

Aspect C9 is the negative electrode composite of any one of aspects C5-C8, wherein M comprises a lanthanide, and wherein the lanthanide is optionally La, Ce, Pr, Nd, Eu, Tb, Dy, Ho, Er, Tm, or Lu.

Aspect C10 is the negative electrode composite of any one of aspects C5-C9, wherein M comprises a post transition metal or a metalloid and wherein the transition metal or metalloid is optionally B, A1, Ga, In, Si, Ge, Sn, Pb, or Te.

Aspect C11 is the negative electrode composite of any one of aspects C5-C10, wherein M comprises Ce, Dy, Re, Nd, Tb, or any combination thereof.

Aspect C12 is the negative electrode composite of any one of aspects C5-C11, wherein the negative electrode active material is single phase as measured by the PXRD Test Method.

Aspect C13 is the negative electrode composite of any one of aspects C5-C12, wherein the negative electrode active material is multiphase as measured by the PXRD Test Method.

Aspect C14 is the negative electrode composite of any one of aspects C5-C13, wherein the electrode composite has a specific capacity at 20° C. to 25° C. of 300 mAh/g or greater, 310 mAh/g or greater, 320 mAh/g or greater, 340 mAh/g or greater, or 350 mAh/g or greater as measured according to the Specific Capacity Test Method.

Aspect C15 is the negative electrode composite of any one of aspects C5-C14, wherein the electrode composite has a specific capacity at 37° C. of 300 mAh/g or greater, 310 mAh/g or greater, 320 mAh/g or greater, 340 mAh/g or greater, or 350 mAh/g or greater as measured according to the Specific Capacity Test Method.

Aspect C16 is the negative electrode composite of any one of aspects C5-C15, wherein the binder comprises polyvinylidene fluoride.

Aspect C17 is the negative electrode composite of any one of aspects C5-C16, wherein the electrically conductive additive comprises carbon.

Aspect C18 is a negative electrode comprising the negative electrode composite of any one of claims 82-98 and a current collector.

Aspect C19 is the negative electrode of aspect C18, wherein the electrode composite has a specific capacity at 20° C. to 25° C. of 300 mAh/g or greater, 310 mAh/g or greater, 320 mAh/g or greater, 340 mAh/g or greater, or 350 mAh/g or greater as measured according to the Specific Capacity Test Method.

Aspect C20 is the negative electrode of any one of aspects C18-C19, wherein the electrode composite has a specific capacity at 37° C. of 300 mAh/g or greater, 310 mAh/g or greater, 320 mAh/g or greater, 340 mAh/g or greater, or 350 mAh/g or greater as measured according to the Specific Capacity Test Method.

Aspect C21 is the negative electrode of any one of aspects C18-C20, wherein the negative electrode has an average discharge voltage at a 20° C. to 25° C. working temperature of 2.2 volts (V) or less, 1.8 V or less, 1.7 V or less, 1.6 V or less, 1.5 V, 1.4 V or less, or 1.2 V or less vs Li/Li+ as measured according to the Average Discharge Test Method.

Aspect C22 is the negative electrode of any one of aspects C18-C21, wherein the negative electrode has an average discharge voltage at a 37° C. working temperature of 2.2 volts (V) or less, 1.8 V or less, 1.7 V or less, 1.6 V or less, 1.5 V or less, 1.4 V or less, or 1.2 V or less vs Li/Li+ as measured according to the Average Discharge Test Method.

Aspect C23 is the negative electrode of any one of aspects C18-C22, wherein the negative electrode has a capacity retention after 10 charge-discharge cycles at a 20° C. to 25° C. working temperature of 80% or greater, 90% or greater, 95% or greater, 97% or greater, 98% or greater, or 99% or greater as measured according to the Capacity Retention Test Method.

Aspect C24 is the negative electrode of any one of aspects C18-C23, wherein the negative electrode composite has an capacity retention after 10 charge-discharge cycles at a 37° C. working temperature of 80% or greater, 90% or greater, 95% or greater, 97% or greater, 98% or greater, or 99% or greater as measured according to the Capacity Retention Test Method.

Aspect C25 is the negative electrode of any one of aspects C18-C24, wherein the negative electrode has a discharge rate retention after 10 charge-discharge cycles at a 20° C. to 25° C. working temperature of 75% or greater, 80% or greater, 85% or greater, 90% or greater, or 95% or greater as measured according to the Rate Retention Test Method.

Aspect C26 is the negative electrode of any one of aspects C18-C25, wherein the negative electrode has a discharge rate retention after 10 charge-discharge cycles at a 37° C. working temperature of 75% or greater, 80% or greater, 85% or greater, 90% or greater, or 95% or greater as measured according to the Rate Retention Test Method.

Aspect C27 is the negative electrode of any one of aspects C18-C26, wherein the electrode composite has a diffusivity at a 20° C. to 25° C. working temperature of 1×10⁻⁹ square centimeters per second (cm²/s) to 5×10⁻⁷ cm²/s or 1×10⁻⁹ cm²/s to 2×10⁻⁷ cm²/s as measured according to the Diffusivity Test Method.

Aspect C28 is the negative electrode of any one of aspects C18-C27, wherein the electrode composite has a diffusivity at a 37° C. working temperature of 1×10⁻⁹ square centimeters per second (cm²/s) to 5×10⁻⁷ cm²/s or 1×10⁻⁹ cm²/s to 2×10⁻⁷ cm²/s as measured according to the Diffusivity Test Method.

Aspect C29 is the negative electrode of any one of aspects C18-C28, wherein the negative electrode has a irreversibility capacity at a 20° C. to 25° C. working temperature of 10% or less, 5% or less, 4% or less, 3% or less, 2% or less, or 1% or less as measured by the Irreversibility Capacity Test Method.

Aspect C30 is the negative electrode of any one of aspects C18-C29, wherein the negative electrode has a irreversibility capacity at a 37° C. working temperature of 10% or less, 5% or less, 4% or less, 3% or less, 2% or less, or 1% or less as measured by the Irreversibility Capacity Test Method.

Aspect C31 is an electrochemical cell comprising:
the negative electrode of any one of aspects C18-C30;
a positive electrode;
a separator between the negative electrode and the positive electrode; and an electrolyte.

Aspect D1 is an implantable medical device comprising a battery comprising: the negative electrode composite of any one of aspects B1-B7 and C1-C17, the negative electrode of any one of aspects B8-B10 and C18-C30, the electrode composite of any one of aspects B11-B21, the electrode of any one of aspects B22-B24, or the electrochemical cell of any one of aspects B25-B46 and C31.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

The following abbreviations may be used in the following examples and/or other places in this disclosure: milliliter; L=liter; m=meter, mm=millimeter, min=minutes; s=seconds; cm=centimeter, kg=kilogram, g=gram, min=minute, s=second, h=hour, ° C.=degrees Celsius, wt-%=weight percent; atom-%=atom percent; RT=room temperature; M=molar; V=volts; A=ampere, mA=milliampere; mAg⁻¹, mA per g, mAh/g=milliampere hour per gram; mg/cm²=milligram per square centimeter; and kV=kilovolts; wt-%=weight percent; w/w=weight by weight; E-x=10^{-x} where x is a number.

### Test Methods

### Specific Capacity Test Method:

Specific Capacity was calculated by integrating the area under the current vs time curve (Idt) and dividing by the mass of active material at a scan rate of 0.1 V/h.

### Average Discharge Voltage Test Method:

The discharge voltage was calculated by integrating IVdt and dividing by the integral of Idt at a scan rate of 0.1 V/h.

### Rate Retention Test Method:

The rate retention was calculated as the discharge capacity of the working electrode at 2 V/h divided by the discharge capacity of the working electrode at 0.1 V/h. Discharge capacity is calculated as by integrating current over time (Idt) during discharge and dividing by the mass of the electrode active material. Charge capacity is calculated as by integrating current over time (Idt) during charging and dividing by the mass of the electrode active material.

### Capacity Retention Test Method:

The capacity retention was calculated as the discharge capacity of the working electrode after the 10^{th} cycle divided by the initial discharge capacity (1^{st} cycle) of the working electrode.

### Irreversible Capacity Test Method:

The irreversible capacity was calculated as the first discharge capacity of the working cell minus the first charge capacity, divided by the first charge capacity multiplied by 100%. Discharge capacity is calculated as by integrating current over time (Idt) during discharge and dividing by the mass of the electrode active material at 0.1 V/h.

### Diffusion Coefficient Test Method:

The diffusion coefficient (D) for the working electrode was calculated using the Randles-Ševčik equation as described in Electrochemica Acta 439, 141665 (2023).

### Example 1—Pseudoternary System

### Pseudoternary System Sample Preparation.

TABLE 1
Stoichiometry of nine compositions discussed herein,
including compositions in the pseudoternary system. Oxygen content
is calculated assuming Ti⁴⁺, W⁶⁺, Nb⁵⁺.

| Sample label | Stoichiometry |
|---|---|
| A1 | Nb_{0.475}W_{0.525}O_{2.863} |
| A2 | Nb_{0.475}W_{0.375}Ti_{0.15}O_{2.713} |
| A3 | Nb_{0.325}W_{0.6}Ti_{0.075}O_{2.73} |
| B1 | Nb_{0.85}W_{0.15}Ti_{0.2}O_{2.675} |
| B2 | Nb_{0.85}W_{0.1}Ti_{0.05}O_{2.625} |
| B3 | Nb_{0.85}W_{0.075}Ti_{0.075}O_{2.5} |
| B4 | Nb_{0.775}W_{0.15}Ti_{0.075}O_{2.638} |
| C1 | Nb_{0.85}Ti_{0.15}O_{2.425} |
| C2 | Nb_{0.6}W_{0.1}Ti_{0.3}O_{2.4} |

Pseudoternary system synthesis and characterization experiments described herein used high-throughput approaches. More specifically, 137 compositions in the pseudoternary system were synthesized and characterized using methods described herein. An illustrative sol-gel method according to aspects described herein was used for the synthesis to afford samples in the pseudoternary system. More specifically, the samples were made by preparing homogenous precursor solutions in alumina cups with 3 M citric acid as a chelating agent and varying volumes of 0.8 M tungsten trioxide, titanium butoxide, and ammonium niobate oxalate hydrate. The homogenous precursor solutions underwent a gelation process by heating at 60° C., 110° C., and 200° C. under vacuum, affording gels. The resulting gels were crushed and then off-gassed at 400° C. for 6 hours, removing nitrates and citrate byproducts that may have been formed in the gelation process. During the off-gassing, the gels were outfitted with an 8×8 aluminum smokestack to prevent or reduce sample cross-contamination. The off-gassed samples were further calcinated for 3 hours in ambient air by heating at a rate of 10° C. per minute to 1,000° C. The calcinated samples were cooled at a rate of 5° C. per minute.

### Pseudoternary System Sample Analysis—X-Ray Diffraction.

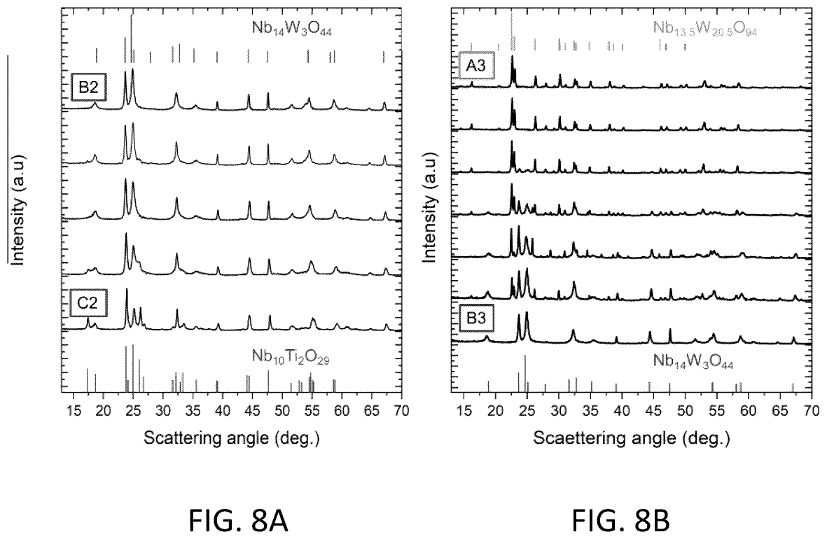

Samples were characterized using PXRD analysis. More specifically, PXRD analysis was used to determine the sample phases. A graphical representation of the phase stability diagram of the illustrative Nb_{1-x-y}TiₓW_{y}O_{z} pseudoternary system based on PXRD analysis is shown on a Gibbs' triangle displaying solid-solution regions in FIG. 7. The phase stability diagram of FIG. 7 includes labels for illustrative samples A1, A2, A3, B1, B2, B3, B4, C1, and C2, which are discussed herein. No clear miscibility gap was observed along the phase transition between the Nb₁₄W₃O₄₄ and Nb₁₀Ti₂O₂₉ phases, as indicated by the dotted line between the Nb₁₄W₃O₄₄ and Nb₁₀Ti₂O₂₉ phases. Graphical representations of PXRD patterns along two compositions lines is shown in FIGS. 8A and 8B. FIG. 8A shows PXRD patterns on compositions lines between samples B2 and C2. FIG. 8B shows PXRD patterns on composition lines between samples A3 and B3.

As shown in FIG. 7, all PXRD patterns were identified as being either single phase or mixtures of phases observed in the pseudobinary systems. Three of the single-phase regions extend well into the ternary region containing each of Nb, W, and Ti, showing a rich chemistry where all three elements may mix on the lattices and offers a broad composition space of interest as potential battery materials and, more specifically, potential electrode active materials.

The phases that extend into the ternary region include Nb_{13.5}W_{20.5}O₉₄ (phase A), Nb₁₄W₃O₄₄ (phase B) and Nb₁₀Ti₂O₂₉ (phase C). Two of these phases, B and C, show no gap between them, as shown in the PXRD patterns in FIG. 8A. With no apparent materials showing two phases co-existing, and without wishing to be bound by theory, this represents a phase transition where the W-rich phase transforms into the Ti-rich phase along the dotted line in FIG. 7. In the layered oxides, as more Li was added into NiO, a point is reached where the Li orders into layers and thereby dramatically improving battery performance. As shown in FIG. 8A, as the Ti metal fraction increases, several peaks appear after the phase transition and all index to the Nb₁₀Ti₂O₂₉ phase. The consequences of this phase transition on the electrochemistry are discussed herein.

With reference again to FIG. 7, the Nb_{13.5}W_{20.5}O₉₄ phase (phase A) features the largest solubility range extending from approximately x=0 to 0.2 and approximately y=0.25 to 0.55 in Nb_{1-x-y}TiₓW_{y}O_{z}. Despite this large solid-solution region, and without wishing to be bound by theory, the lattice parameters do not change appreciably due to the nearly identical ionic radii. This chemical flexibility is reflected in the PXRD crystallographic information files (cif) used to identify the phases. For example, the PXRD cif for Nb_{13.5}W_{20.5}O₉₄ shows that all 8 Nb-containing sites have mixed occupation wherein at least 16 at. % W is present (4 sites have an approximate 40/60 split). Without wishing to be bound by theory, the high degree of site mixing may be conducive to solid-solutions and Ti may replace equally Nb and W in this structure. As the Nb content of the material increases, this phase eventually co-exists with the Nb₁₄W₃O₄₄ phase until the Nb₁₄W₃O₄₄ phase forms its own solid solution region (see FIG. 7). This progression is shown in FIG. 8B, where a set of patterns along a composition line confirms both single-phase regions and demonstrates that co-existence occurs in between. Generally, and without wishing to be bound by theory, single-phase materials are the most desirable for battery electrode active materials, as synergy between phases is uncommon. Thus, and still without wishing to be bound by theory, the wide composition spaces where single phases are afforded offer a wide variety of suitable and potentially suitable electrode materials.

The three crystal structures of interest are shown in FIGS. 6A-6C. Phases B (shown in FIG. 6B) and C (shown in FIG. 6C) are similar in structure as is suggested by their PXRD patterns, but contain a few noted differences. While all metal sites in phase C are mixed Nb/Ti octahedral sites, all Nb sites are again mixed Nb/W octahedra in phase B, but there are also W occupying tetrahedral sites. Without wishing to be bound by theory, the phase transition between phase B and phase C described herein (illustrated in FIG. 8A) occurs when the pseutoternary compound includes sufficient Ti to force the W site to convert to an octahedral environment. It is also of note that phase A contains all metals (Nb/W in the PXRD cif represented in FIG. 8B) in octahedral environments only, with a combination of corner and edge sharing, similar to that seen in phase C.

Pseudoternary System Sample Analysis—Scanning Electron Microscopy (sEM).

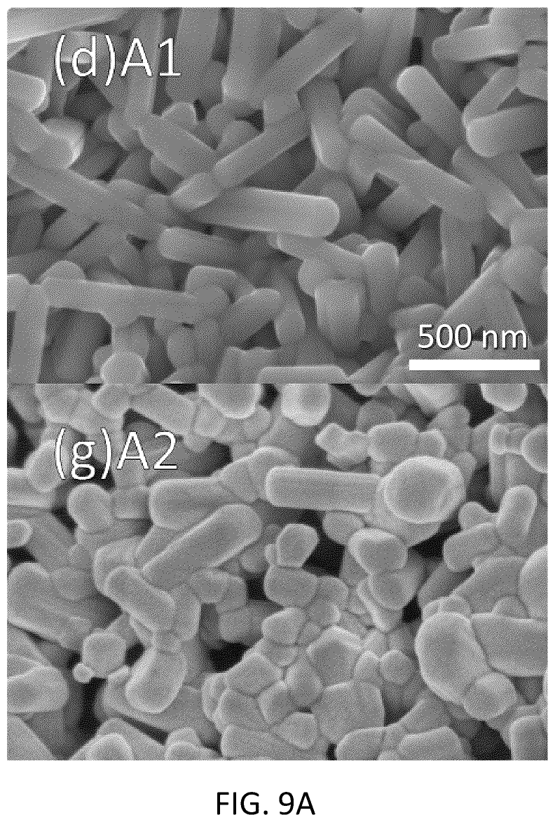

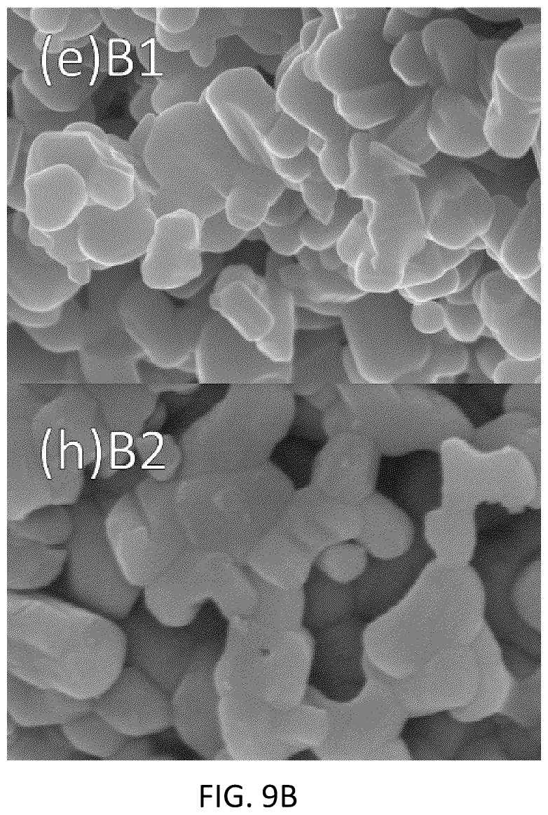

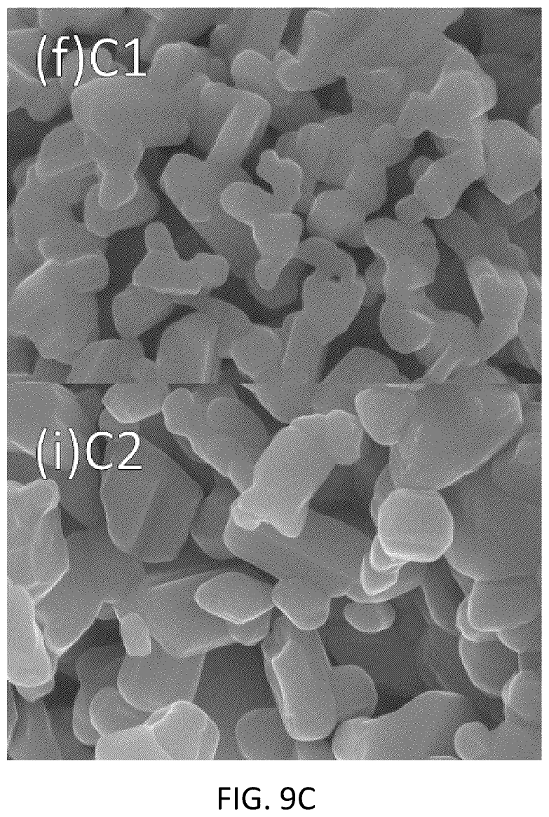

Without wishing to be bound by theory, the morphologies of primary particles may generally have significant impact on electrochemical performance. As such, SEM was used to image six representative materials. SEM images of three illustrative pseudoternary compounds—samples A2, B2, and C2—within single—phase regions within the ternary space (see FIG. 7) and three corresponding pseudobinary compounds—samples A1, B1, and C1—are shown in FIGS. 9A-9C. The SEM images demonstrate that the morphologies are similar in samples of the same crystal structure. More specifically, as shown in FIG. 9A, samples A1 and A2, both take the Nb_{13.5}W_{20.5}O₉₄ crystal structure and show rodlike primary particles on the scale of about 600 nanometers (nm) length. There is a change apparent in these rods when the pseudoternary compound is formed by including Ti in sample A2. The particle morphologies of sample A2 is generally shorter in length than the particle, or crystal, morphologies of sample A1, with some sample A2 particles even appearing to be nearly spherical and overall the particles being smaller.

By contrast, and with reference to FIGS. 9B and 9C, the particle morphologies of the Nb₁₄W₃O₄₄ (phase B, see FIG. 9B) and Nb₁₀Ti₂O₂₉ (phase C, see FIG. 9C) phases are all similar to each other and there is little or no apparent influence on morphology as a result of including the third metal fraction into the materials. Without wishing to be bound by theory, the similarities in these morphologies mirror the similarities in the structures as shown in the PXRD patterns where a phase transition occurred between these two structures.

### Pseudoternary System Samples Analysis—Electrochemical Analysis.

After PXRD, each sample underwent electrochemical testing. Both a high and low loading method were used for electrode fabrication. Combinatorial cells with 8×8 samples as electrode materials were used to test electrochemical properties. First, a custom-designed printed circuit board (PCB, Optima Tech) with 64 nickel (Ni) pads was used. To form the electrodes, a slurry of approximately 11 wt. % carbon black and 5 wt. % of PVDF and 0.3 mg of active material was drop casted onto the PCB. This yielded a mass loading of approximately 2.5 mg/cm². The PCB and electrodes were then dried at 80° C. overnight to evaporate N-Methylpyrrolidone (NMP). The assembly of the combinatorial cell was performed in an argon-filled glovebox. The electrolyte was 1 M LiPF₆ in 1:1 ethylene carbonate:dimethyl carbonate (EC:DMC) (SOULBRAIN MI). Lithium metal foil was used as the counter electrode with two WHATMAN GF/D glass microfiber separators. The cell was then sealed using a double-sided sealing tape. Cyclic voltammetry (CV) was performed with the voltage range 1.0 to 3.0 V vs. Li/Li+ at a variety of scan rates on a lab-built high-throughput electrochemical system which utilizes a quad voltage source (KEITHLEY 213) and a KEITHLEY 2750 multichannel voltmeter. 64 CVs were performed simultaneously. Electrochemical testing was also performed at 37° C. in a house-designed temperature-controlled apparatus. Data were processed to extract average voltages, and specific capacities over the multiple cycles performed.

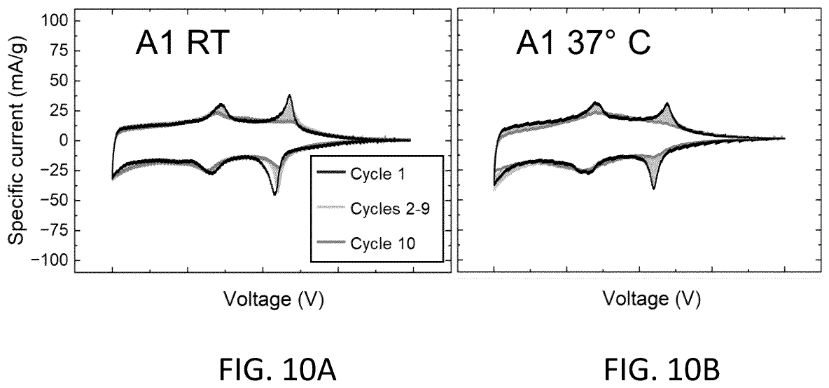

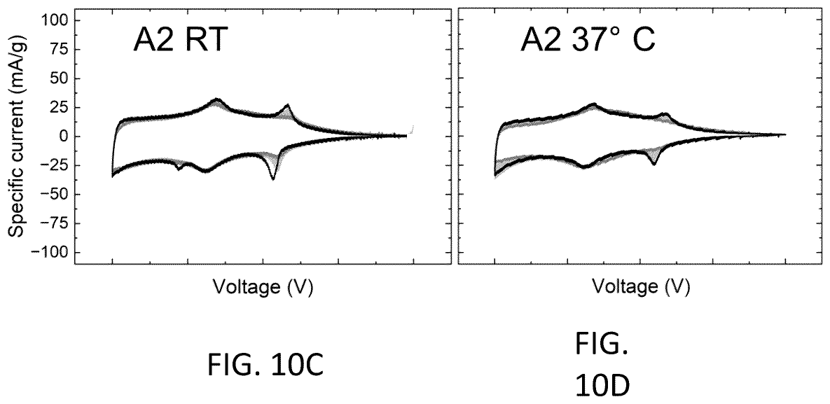

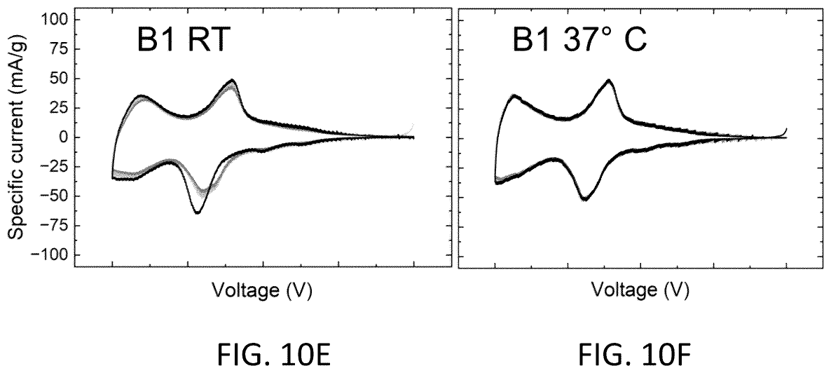

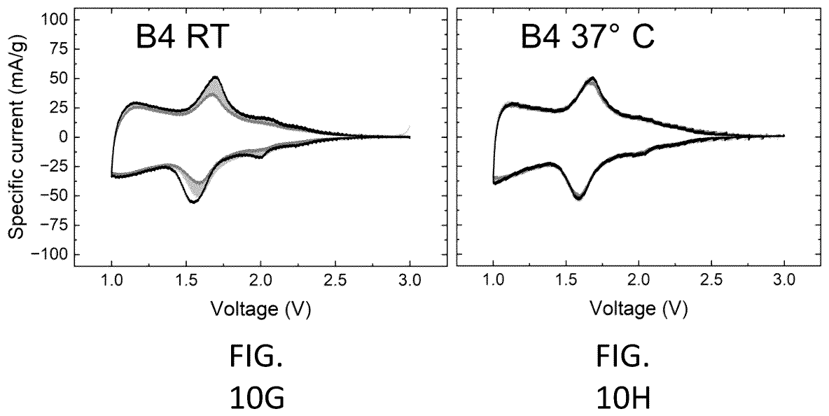

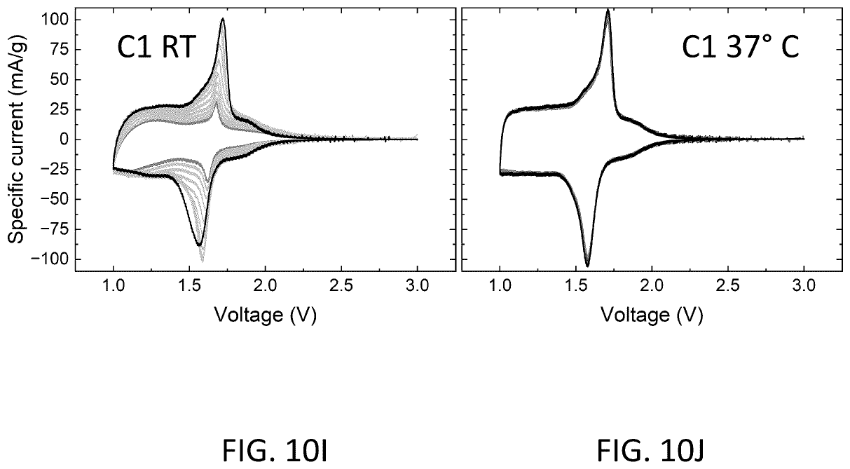

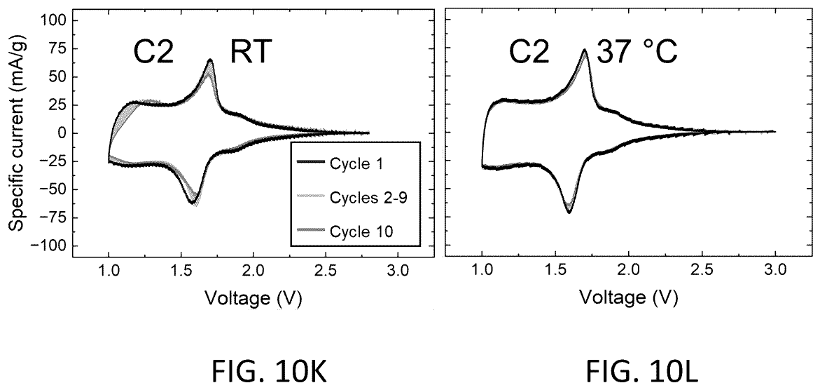

To investigate the electrochemical performance of the samples in the pseudoternary, CV was performed with Li metal used as the counter electrode. Graphical representations of CVs are shown in FIGS. 10A-10L. Each CV shows 10 cycles at 0.1 V/h between 1 and 3 V. Graphical representation of CVs are shown for sample A1 (pseudobinary) at ambient room temperature in FIG. 10A and at 37° C. ambient temperature in FIG. 10B, sample A2 (pseudoternary) at ambient room temperature in FIG. 10C and at 37° C. ambient temperature in FIG. 10D, sample B1 (pseudobinary) at ambient room temperature in FIG. 10E and at 37° C. ambient temperature in FIG. 10F, sample B4 (pseudoternary) at ambient room temperature in FIG. 10G and at 37° C. ambient temperature in FIG. 10H, sample C1 (pseudobinary) at ambient room temperature in FIG. 10I and at 37° C. ambient temperature in FIG. 10J, and sample C2 (pseudotermary) at ambient room temperature in FIG. 10K and at 37° C. ambient temperature in FIG. 10L.

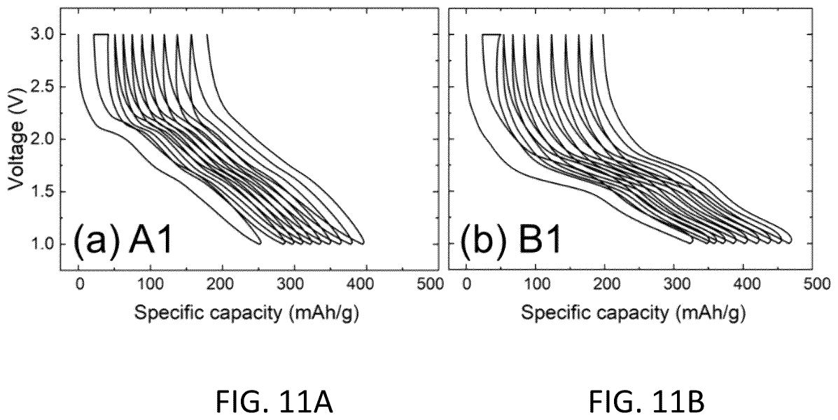

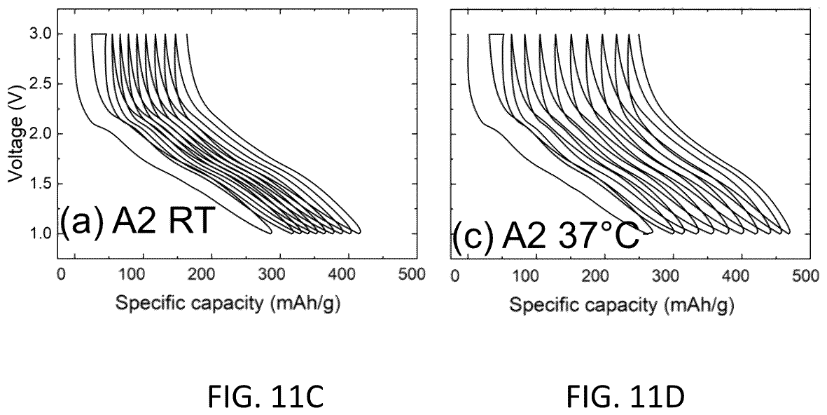

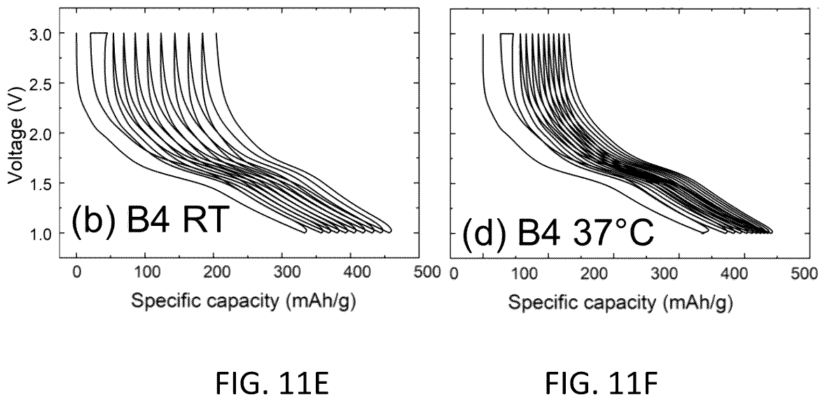

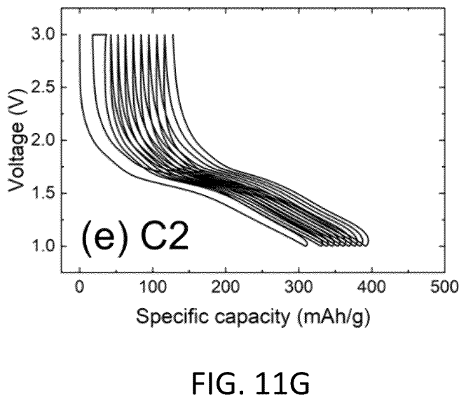

Additionally, voltage curves were characterized for each of samples A1, A2, B1, B4, and C2 with Li metal used as the counter electrode. Graphical representations of voltage curves for samples over 10 cycles at 0.1 V/h are shown in FIGS. 11A-11G. Graphical representations of voltage curves are shown for sample A1 (pseudobinary) at ambient room temperature in FIG. 11A, sample B1 (pseudobinary) at ambient room temperature in FIG. 11B, sample A2 (pseudoternary) at ambient room temperature in FIG. 11C and at 37° C. ambient temperature in FIG. 11D, sample B4 (pseudoternary) at ambient room temperature in FIG. 11E and at 37° C. ambient temperature in FIG. 11F, and sample C2 (pseudoternary) at ambient room temperature in FIG. 11G.

The CVs and voltage curves at ambient room temperature for the pseudoternary samples (e.g., A2, B4, and C2) are qualitatively the same as those for the pseudobinary samples (e.g., A1, B1, and C1). While the CVs of the pseudoternary samples vary in shape, they are largely identical for each phase (e.g., A1 and A2 generally match in shape, as do B1 and B4, and C1 and C2).

Without wishing to be bound by theory, the CVs and voltage curves show that, of the materials characterized, the phase A materials show quality extended cycling at ambient room temperature with the pseudoternary material A2 showing improved extended cycle compared with the pseudobinary A1. In comparison, and still without wishing to be bound by theory, each of the phase B and C materials show dramatically improved extended cycling at the elevated ambient temperature of 37° C.

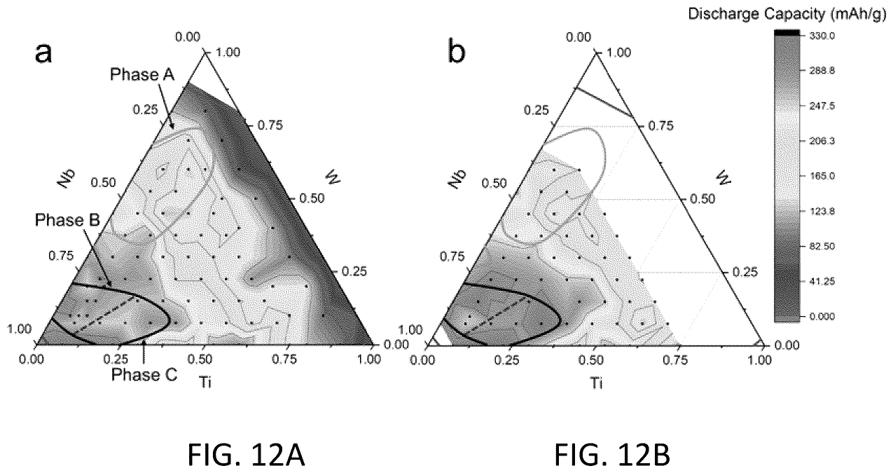

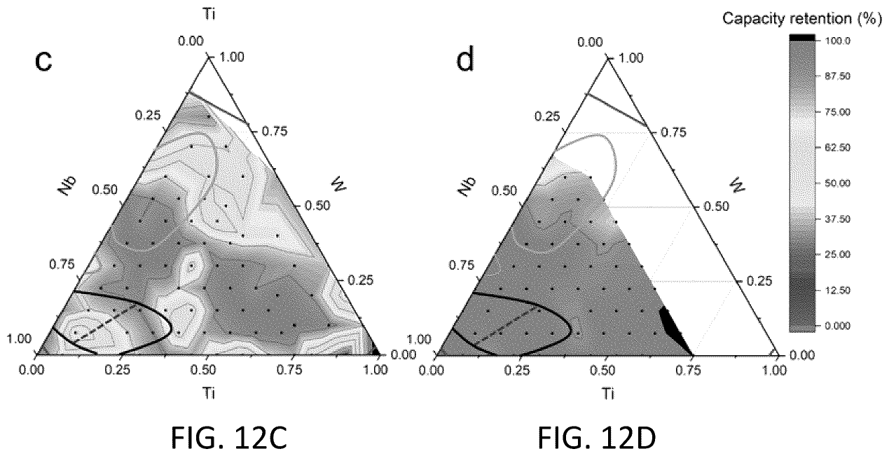

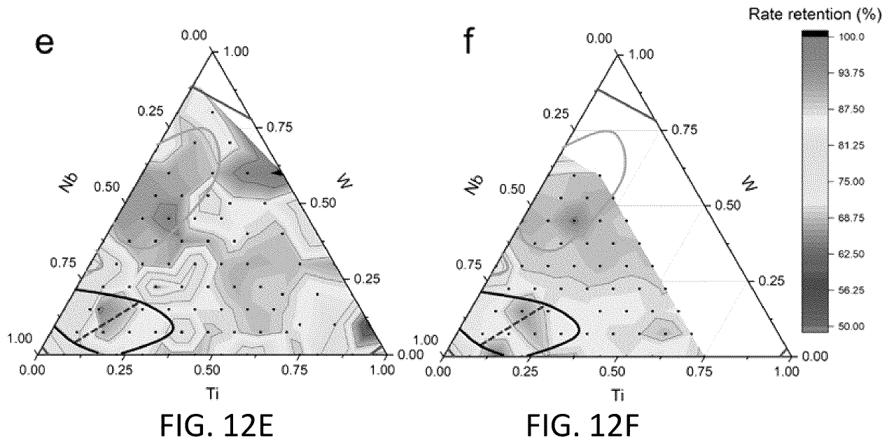

To generally characterize electrochemical performance metrics based on stoichiometry, or relative metal fractions, graphical representations of metrics from the electrochemical testing are shown in FIGS. 12A-12F and plotted over the Gibbs' triangles of FIG. 7 along with the boundaries of the A, B and C single-phase regions.

Graphical representations of first discharge capacities are shown at ambient room temperature in FIG. 12A and at 37° C. ambient temperature in FIG. 12B. Graphical representations of energy capacity retention after 10 cycles are shown at ambient room temperature in FIG. 12C and at 37° C. ambient temperature in FIG. 12D.

Graphical representations of discharge rate retention are shown at ambient room temperature in FIG. 12E and at 37° C. ambient temperature in FIG. 12F. The rate retention test was performed at various sweep rates after the first cycle was calculated as the discharge capacity obtained at 1 V/h divided by the discharge capacity on the first cycle at 0.1 V/h. Additionally, the selected samples of each phase demonstrating the preferred electrochemical performance metrics are summarized in Table 2.

TABLE 2
electrochemical performance metrics of selected samples

| | | | Average | | | |
|---|---|---|---|---|---|---|
| | | | discharge | Capacity | Rate | |
| | Temper- | Capacity | voltage | retention | retention | |
| Sample | ature | (mAh/g) | (V) | (%) | (%) | D (cm²/s) |
| A1 | RT | 231.9 | 1.78 | 94.9 | 92.3 | 6.28 × 10⁻⁸ |
| | 37° C. | 250.9 | 1.77 | 86.1 | 88.9 | 6.07 × 10⁻⁸ |
| A2 | RT | 263.0 | 1.71 | 97.0 | 89.3 | 7.73 × 10⁻⁸ |
| | 37° C. | 238.9 | 1.74 | 93.9 | 88.0 | 6.00 × 10⁻⁸ |
| B1 | RT | 304.2 | 1.65 | 89.8 | 78.5 | 1.26 × 10⁻⁷ |
| | 37° C. | 295.6 | 1.64 | 98.9 | 83.2 | 1.28 × 10⁻⁷ |
| B4 | RT | 315.3 | 1.63 | 81.4 | 79.4 | 1.30 × 10⁻⁷ |
| | 37° C. | 318.2 | 1.64 | 98.7 | 85.4 | 1.44 × 10⁻⁷ |
| C1 | RT | 310.4 | 1.57 | — | 85.7 | 1.65 × 10⁻⁷ |
| | 37° C. | 325.5 | 1.56 | 97.2 | 83.9 | 2.25 × 10⁻⁷ |
| C2 | RT | 293.9 | 1.59 | 91.5 | 83.1 | 1.28 × 10⁻⁷ |
| | 37° C. | 329.0 | 1.59 | 97.2 | 84.3 | 2.10 × 10⁻⁷ |

As shown in FIG. 12A and Table 2, the materials with higher first discharge capacities are found in phases B and C, and, generally, in the region of the pseudoternary near the Nb corner (i.e., having a higher Nb metal fraction). As shown in FIG. 12B, first discharge capacities are generally increased at 37° C. ambient temperature compared with ambient room temperature. Without wishing to be bound by theory, this shows that Nb content influences the electrochemical performance metrics. Both phases B and C materials are found with generally high first discharge capacities in this region. First discharge capacities of materials in this region may be up to approximately 315 mAh/g at ambient room temperature, and many pseudoternary compositions with high Nb content demonstrate first discharge capacities of 300 mAh/g or greater, which approaches the theoretical capacities in these materials. This is generally an improvement over the pseudobinary compositions. The region with relatively higher ambient room temperature discharge capacities spans the gap between the A and B phases and also includes two samples that are only phase A. Without wishing to be bound by theory, a benefit of these two phase A materials may be that they show high capacity retentions after 10 cycles at ambient room temperature of 94.9 and 97%. By comparison, the phase B and C materials may show generally lower retentions, such as no higher than 91%.

As shown in Table 2, and without wishing to be bound by theory, the phase A materials generally show the best overall performance at room temperature. As described herein, electrochemical performance at 37° C. ambient temperature, or approximately body temperature, is a desirable characteristic for implantable devices. As best shown in FIG. 12B, the first cycle discharge capacities may be generally unchanged when cells are operated at 37° C. ambient temperature. More specifically, compositions with higher Nb content (such as phases B and C) may generally demonstrate a slight increase in capacity, such as up to approximately 318.2 mAh/g. In comparison, compositions with lower Nb content (such as phase A) may generally demonstrate a minor decrease.

Without wishing to be bound by theory, electrochemical performance metrics may generally be reduced for the phase A materials at 37° C. ambient temperature compared with the phase A materials at ambient room temperature. In comparison, phases B and C both generally demonstrate improved electrochemical performance metrics at 37° C. ambient temperature compared with the same materials at ambient room temperature. Without wishing to be bound by theory, the largest benefit may be the improvement in extended cycling, such as shown in FIG. 12D in the high-Nb region of phases B and C. As shown in FIG. 12D, the high-Nb region of phases B and C demonstrates generally no deterioration of any measured electrochemical performance metric.

As shown in Table 2, samples B4 and C2 (both in the pseudoternary system) may show the preferred electrochemical performance at 37° C. ambient temperature in terms of capacity and retention. Without wishing to be bound by theory, capacity and retention at 37° C. ambient temperature may be particularly desirable for use in implantable devices. Furthermore, and still without wishing to be bound by theory, rate performance is also desirable for high-power applications.

As shown in FIG. 12E, several materials, such as sample B2, demonstrate improved rate performance with capacities of approximately 85% after increasing the rate by an order of magnitude. The rate retention of sample B4 is slightly higher than that of sample C2, such that sample B4 may be described as generally having the best electrochemical performance at 37° C. ambient temperature, as summarized in Table 2.

### Example 2—Doped TNO

### Test Half Cell:

For each electrochemical test method described in Example 2, the negative electrode active material was configured in a half cell. Each half cell included a working electrodemass loading of approximately 2.5 mg/cm², where the working electrode included 11 wt-% carbon black, 5 wt-% of PVDF, and 0.3 mg (84 wt-%) of active electrode active material (TNO or doped-TNO). Lithium foil was used as the counter electrode and was separated. The half-cell included two glass microfiber separators. The electrolyte included 1 M lithium hexafluoride (LiPF₆) in 1:1 (wt/wt) ethylene carbonate (EC):dimethyl carbonate (DMC).

More specifically the samples of Example 2 were tested in combinatorial cells Combinatorial cells with 8 by 8 samples as electrode materials were used to test electrochemical properties of the samples, as described in detail in Potts et al (Potts, K. P.; Grignon, E.; McCalla, E. Accelerated Screening of High-Energy Lithium-Ion Battery Cathodes. ACS Applied Energy Materials 2019, 2 (12), 8388-8393. DOI: 10.1021/acsaem.9b01887), and Adhikari et al (Adhikari, T.; Hebert, A.; Adamic, M.; Yao, J.; Potts, K.; McCalla, E. Development of high-throughput methods for sodium-ion battery cathodes. ACS combinatorial science 2020, 22 (6), 311-318). First, a printed circuit board (PCB, Optima Tech) with 64 Ni pads was used. To create the electrodes, a slurry of approximately 11 wt-% carbon black, 5 wt-% of PVDF, 0.3 mg of active material (doped TNO sample) was drop casted onto the PCB as described in Jia et al (Jia, S.; Yao, E.; Peng, R.; Jonderian, A.; Abdolhosseini, M.; McCalla, E. Chemical Speed Dating: The Impact of 52 Dopants in Na—Mn—O Cathodes. Chemistry of Materials 2022, 34 (24), 11047-11061) and Jonderian et al (Jonderian, A.; Jia, S.; Yoon, G.; Cozea, V. T.; Galabi, N. Z.; Ma, S. B.; McCalla, E. Accelerated Development of High Voltage Li-Ion Cathodes. Advanced Energy Materials 2022, 2201704-2201704. DOI: 10.1002/aenm.202201704) to yield a mass loading of approximately 2.5 mg/cm². The PCB and electrodes were then dried at 50° C. overnight to evaporate n-methyl-2-pyrrolidone (NMP). The assembly of the combinatorial half cell was performed in an argon-filled glovebox. The electrolyte was 1 M LiPF₆ in 1:1 EC:DMC (SoulBrain MI). Li metal foil was used as the counter electrode with two Whatman GF/D glass microfiber separators. The cell was then sealed using a 3M double-sided sealing tape as described in Potts et al (Potts, K. P.; Grignon, E.; McCalla, E. Accelerated Screening of High-Energy Lithium-Ion Battery Cathodes. ACS Applied Energy Materials 2019, 2 (12), 8388-8393. DOI: 10.1021/acsaem.9b01887).

### Cyclic Voltametry:

Various electrochemical properties in Example 2 were determined using cyclic voltammetry. The specific test methods indicated how each parameter was determined.

Cyclic voltammetry (CV) in Example 2 was performed with the voltage range 1.0 V to 3.0 V vs. Li/Li+ at a variety of scan rates using a quad voltage source (KEITHLEY 213) and a Keithley 2750 multichannel voltmeter. 64 CVs were performed simultaneously. Materials were cycled at five different rates: 0.1 V h⁻¹, 0.2 V h⁻¹, 0.5 V h⁻¹, 1 V h⁻¹ and 2 V h⁻¹. After the rate testing, the materials were then cycled at 0.1 V h⁻¹ for 10 full cycles (400 hours or about 17 days of cycling). Electrochemical testing was also performed at 37° C. in a temperature-controlled apparatus. The data was processed to extract average voltages and specific capacities over the multiple cycles performed. For the cycling experiments, a single protocol was used for the extraction of the key electrochemical parameters as shown in Rehman et al (Rehman, S.; Sieffert, J. M.; Lang, C. J.; McCalla, E. NbyW1−yOz and NbxTi1−xOz pseudobinaries as anodes for Li-ion batteries. Electrochimica Acta 2023, 439. DOI: 10.1016/j.electacta.2022.141665).

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

A set of doped TNO samples having the composition Ti_{0.98}Nb_{1.96}M_{0.06}O₇ were prepared where M was one of 52 different elemental dopants (M=B, Na, Mg, A1, Si, P, K, Ca, Sc, V, Cr, Mn, Fe, Ni, Cu, Zn, Ga, Sn, P, Ge, Rb, Sr, Y, Zr, Mo, Rh, Pd, Ag, Cd, In, Te, Cs, Ba, La, Co, Ce, Pr, Nd, Eu, Tb, Dy, Ho, Er, Tm, Lu, Hf, W, Re, Ir, Pt, Au, and Pb). The selected composition represents a 2 atom-% substitution of Ti and Nb with the dopants M. The 2 atom-% dopant concentration was chosen to represent possible dopant percolation in next-nearest neighbor lattice networks. For some dopants, additional compositions were explored. Both the physical properties and electrochemical properties of the doped TNO materials were analyzed.

### TNO Sample Preparation:

All undoped and doped TNO samples were prepared via the sol-gel method described in Rehman et al., (Rehman, S.; Sieffert, J. M.; Lang, C. J.; McCalla, E. NbyW1−yOz and NbₓTi₁₋ₓO_{z} pseudobinaries as anodes for Li-ion batteries. Electrochimica Acta 2023, 439. DOI: 10.1016/j.electacta.2022.141665). Specifically, the samples were made by first dispensing 0.55 M titanium butoxide and ammonium niobate oxalate hydrate
along with stoichiometric quantities of dopants of different precursor concentrations into alumina cups. The B, Na, Mg, A1, Si, P, K, Ca, Sc, V, Cr, Mn, Fe, Ni, Cu, Zn, Ga, Sn, P, Ge, Rb, Sr, Y, Zr, Mo, Rh, Pd, Ag, Cd, In, Te, Cs, Ba, La, Co, Ce, Pr, Nd, Eu, Tb, Dy, Ho, Er, Tm, Lu, Hf, W, Re, Ir, Pt, Au, and Pb dopant precursors are the same as those used in Jonderian et al (Jonderian, A.; Peng, R.; Davies, D.; McCalla, E. Benefits and Limitations of 226 Substitutions into Li—La—Ti—O Perovskites. Chemistry of Materials 2023, 35 (16), 6227-6234). Two different vanadium precursors were used denoted as V and V5 in various figures. Citric acid (3 M) was added as a chelating agent. The homogenous precursor solutions underwent a gelation process by heating at 60° C., 110° C., and finally 200° C. under vacuum. The resulting gels were crushed, then outfitted with an eight by eight aluminum smokestack to prevent sample cross-contamination and off-gassed at 400° C. for 6 hours to remove nitrates and citrate bi-products formed in the gelation process. After removing the smokestack, the samples were further calcined at 1000° C.

Doped TNO samples may be referred to as M-doped TNO of M-TNO where M is the dopant. Undoped TNO samples may be referred to as TNO, undoped TNO, Und, or Und-TMO.

### Example 2 Results

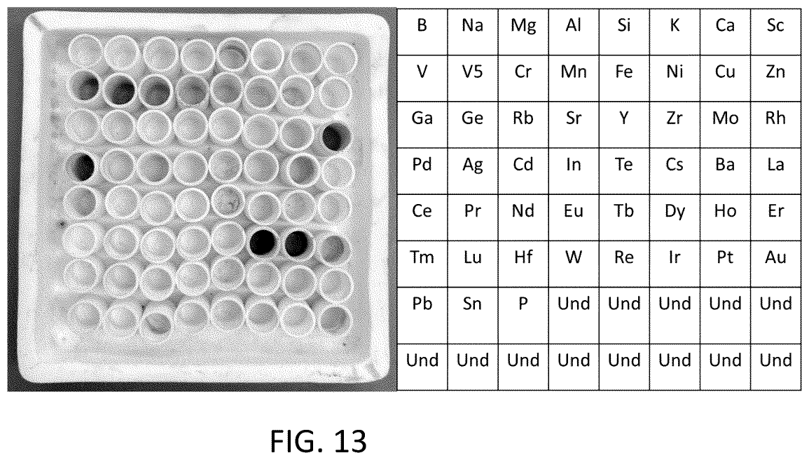

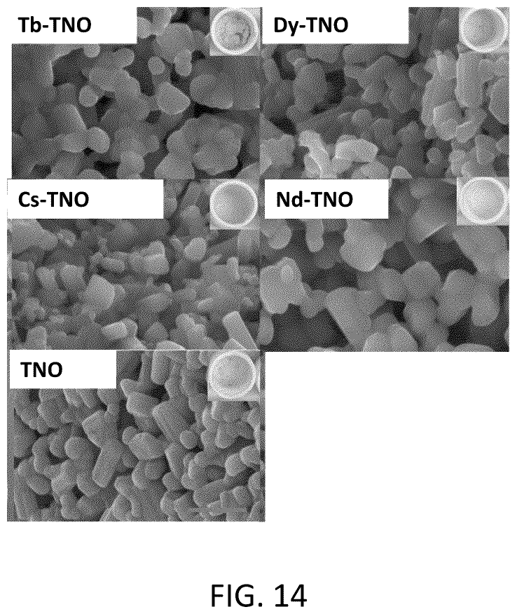

The doped TNO and undoped TNO samples were visual inspected, and the morphology investigated using scanning electron microscopy (SEM). As seen in FIG. 13, some samples have dramatically different colors than the undoped samples (Und is undoped TNO). FIG. 14 shows some representative SEM images of both doped (Tb-TNO, Dy-TNO, Cs-TNO, and Nd-TNO) and undoped TNO. Particle size in Nb based anodes, such as TNO anodes, has previously been shown to affect the electrochemical performance of a material, particularly in influencing its propensity for cracking. SEM revealed several particle morphologies in the undoped TNO, which can also be found in the doped TNO samples. Rodlike particles with a length of 300 nm to 500 nm are the primary morphology found in the undoped TNO material, and also feature prominently in the Cs-TNO and to a lesser extent in Nd-TNO and Dy-TNO materials. More spherical particles with diameters from 100 nm to 300 nm are also common in the materials, particularly in the Tb-TNO sample. The Cs-TNO sample includes marginally smaller particles compared to TNO. There does not seem to be a significant change in particle morphology brought on by the inclusion of a dopant in TNO. Therefore, the differences between undoped TNO and doped TNO are not attribute to morphological changes.

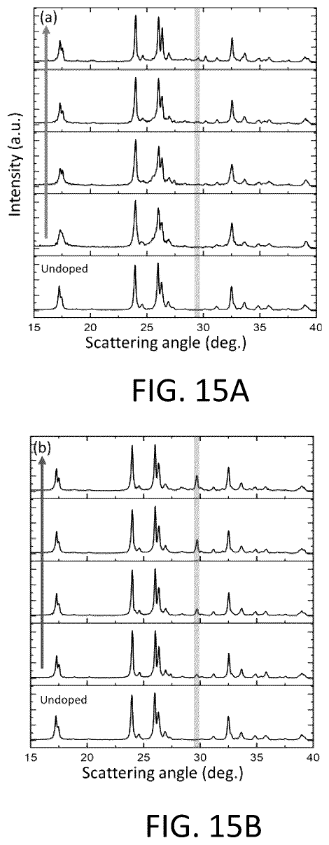

The PXRD Test method was used to analyze the phase of the doped and undoped TNO samples. FIGS. 15A and 15B show representative PXRD patterns of both single-phase doped TNO (e.g., Nd-TNO, FIG. 15A) and multiphase doped TNO (e.g., Tb-TNO, FIG. 15B) materials. At 2 atom-% dopant stoichiometry, 24 of the doped materials synthesized exhibited a single phase TNO structure (Table 3). Twenty-eight doped TNO samples included a secondary phase. The determination of whether a sample is single or multiphase is accomplished by performing single-phase Pawley fits. After performing the Pawley fit, it then becomes easy to identify any peaks coming from secondary phases. The result of this analysis for all M_{0.06}Ti_{0.98}Nb_{1.96}O₇ samples as well as the extracted lattice parameters are included in Table 3.

TABLE 3
PXRD results for the initial dopant screen

| | | | | | Single |
|---|---|---|---|---|---|
| | | | | Unit cell | or Multi |
| Sample | Cell a [A] | Cell b [A] | Cell c [A] | volume[A³] | Phase |
| B_{0.06}Ti_{0.98}Nb_{1.96}O₇ | 20.3577 | 3.798063 | 11.90263 | 794.3376393 | Single |
| Na_{0.06}Ti_{0.98}Nb_{1.96}O₇ | 20.35191 | 3.79815 | 11.88837 | 793.6748499 | Single |
| Mg_{0.06}Ti_{0.98}Nb_{1.96}O₇ | 20.38363 | 3.800095 | 11.91519 | 796.6860925 | Single |
| Al_{0.06}Ti_{0.98}Nb_{1.96}O₇ | 20.36068 | 3.797351 | 11.90768 | 794.4310521 | Multi |
| Si_{0.06}Ti_{0.98}Nb_{1.96}O₇ | 20.36069 | 3.797666 | 11.90857 | 794.534834 | Single |
| P_{0.06}Ti_{0.98}Nb_{1.96}O₇ | 20.36904 | 3.796153 | 11.91371 | 794.809919 | Single |
| K_{0.06}Ti_{0.98}Nb_{1.96}O₇ | 20.35268 | 3.797182 | 11.88894 | 793.6016867 | Single |
| Ca_{0.06}Ti_{0.98}Nb_{1.96}O₇ | 20.36145 | 3.797426 | 11.90703 | 794.5182408 | Multi |
| Sc_{0.06}Ti_{0.98}Nb_{1.96}O₇ | 20.35875 | 3.796893 | 11.90689 | 794.2933126 | Single |
| V_{0.06}Ti_{0.98}Nb_{1.96}O₇ | 20.35491 | 3.795364 | 11.88972 | 793.6103265 | Single |
| V_{0.06}Ti_{0.98}Nb_{1.96}O₇ | 20.36255 | 3.797962 | 11.90331 | 794.8226566 | Single |
| Cr_{0.06}Ti_{0.98}Nb_{1.96}O₇ | 20.4357 | 3.801653 | 11.92803 | 797.700916 | Single |
| Mn_{0.06}Ti_{0.98}Nb_{1.96}O₇ | 20.37814 | 3.799537 | 11.92122 | 796.2661195 | Multi |
| Fe_{0.06}Ti_{0.98}Nb_{1.96}O₇ | 20.38083 | 3.800402 | 11.92073 | 796.6764486 | Single |
| Co_{0.06}Ti_{0.98}Nb_{1.96}O₇ | 20.43572 | 3.797563 | 11.91302 | 796.3126163 | Multi |
| Ni_{0.06}Ti_{0.98}Nb_{1.96}O₇ | 20.38038 | 3.800028 | 11.90662 | 795.7570103 | Single |
| Cu_{0.06}Ti_{0.98}Nb_{1.96}O₇ | 20.37509 | 3.800451 | 11.89654 | 795.7700553 | Single |
| Zn_{0.06}Ti_{0.98}Nb_{1.96}O₇ | 20.37845 | 3.800589 | 11.9104 | 796.4411981 | Single |
| Ga_{0.06}Ti_{0.98}Nb_{1.96}O₇ | 20.37253 | 3.799001 | 11.91591 | 795.7274699 | Multi |
| Ge_{0.06}Ti_{0.98}Nb_{1.96}O₇ | 20.36804 | 3.79823 | 11.9145 | 795.2413027 | Single |
| Rb_{0.06}Ti_{0.98}Nb_{1.96}O₇ | 20.3642 | 3.798384 | 11.90014 | 794.6299195 | Multi |
| Sr_{0.06}Ti_{0.98}Nb_{1.96}O₇ | 20.3687 | 3.797162 | 11.89945 | 794.8078436 | Multi |
| Y_{0.06}Ti_{0.98}Nb_{1.96}O₇ | 20.36997 | 3.800311 | 11.9116 | 795.6724984 | Multi |
| Zr_{0.06}Ti_{0.98}Nb_{1.96}O₇ | 20.37419 | 3.798802 | 11.92128 | 796.1055812 | Single |
| Mo_{0.06}Ti_{0.98}Nb_{1.96}O₇ | 20.35131 | 3.795787 | 11.88732 | 793.0225928 | Single |
| Rh_{0.06}Ti_{0.98}Nb_{1.96}O₇ | 20.44176 | 3.819026 | 11.92762 | 800.7477636 | Multi |
| Pd_{0.06}Ti_{0.98}Nb_{1.96}O₇ | 20.37127 | 3.802015 | 11.89656 | 795.2002953 | Multi |
| Ag_{0.06}Ti_{0.98}Nb_{1.96}O₇ | 20.36185 | 3.799025 | 11.89624 | 794.6286217 | Single |
| Cd_{0.06}Ti_{0.98}Nb_{1.96}O₇ | 20.36493 | 3.798403 | 11.90705 | 795.0078566 | Multi |
| In_{0.06}Ti_{0.98}Nb_{1.96}O₇ | 20.38186 | 3.799922 | 11.9223 | 796.7158929 | Multi |
| Sn_{0.06}Ti_{0.98}Nb_{1.96}O₇ | 20.38839 | 3.801089 | 11.91772 | 797.2641796 | Single |
| Te_{0.06}Ti_{0.98}Nb_{1.96}O₇ | 20.37131 | 3.80007 | 11.90756 | 795.7516489 | Multi |
| Cs_{0.06}Ti_{0.98}Nb_{1.96}O₇ | 20.36636 | 3.798851 | 11.90163 | 794.9122248 | Multi |
| Ba_{0.06}Ti_{0.98}Nb_{1.96}O₇ | 20.38023 | 3.800707 | 11.89888 | 795.6347341 | Single |
| La_{0.06}Ti_{0.98}Nb_{1.96}O₇ | 20.36732 | 3.797493 | 11.91422 | 795.0649632 | Multi |
| Ce_{0.06}Ti_{0.98}Nb_{1.96}O₇ | 20.37226 | 3.798404 | 11.91359 | 795.6139338 | Multi |
| Pr_{0.06}Ti_{0.98}Nb_{1.96}O₇ | 20.36946 | 3.798118 | 11.91375 | 795.3335829 | Multi |
| Nd_{0.06}Ti_{0.98}Nb_{1.96}O₇ | 20.35962 | 3.797303 | 11.90619 | 794.5055976 | Multi |
| Eu_{0.06}Ti_{0.98}Nb_{1.96}O₇ | 20.37052 | 3.799134 | 11.9132 | 795.7408275 | Multi |
| Tb_{0.06}Ti_{0.98}Nb_{1.96}O₇ | 20.37518 | 3.798975 | 11.91485 | 796.0142576 | Multi |
| Dy_{0.06}Ti_{0.98}Nb_{1.96}O₇ | 20.3786 | 3.799366 | 11.91543 | 796.2537755 | Multi |
| Ho_{0.06}Ti_{0.98}Nb_{1.96}O₇ | 20.36217 | 3.796846 | 11.90964 | 794.472399 | Multi |
| Er_{0.06}Ti_{0.98}Nb_{1.96}O₇ | 20.36467 | 3.797961 | 11.91446 | 795.186327 | Multi |
| Tm_{0.06}Ti_{0.98}Nb_{1.96}O₇ | 20.3627 | 3.798405 | 11.91069 | 795.0379168 | Multi |
| Lu_{0.06}Ti_{0.98}Nb_{1.96}O₇ | 20.37531 | 3.80091 | 11.91343 | 796.3612589 | Multi |
| Hf_{0.06}Ti_{0.98}Nb_{1.96}O₇ | 20.4093 | 3.802326 | 11.93194 | 799.1813464 | Single |
| W_{0.06}Ti_{0.98}Nb_{1.96}O₇ | 20.36956 | 3.799879 | 11.9056 | 795.2949653 | Single |
| Re_{0.06}Ti_{0.98}Nb_{1.96}O₇ | 20.36956 | 3.799487 | 11.9096 | 795.6586108 | Multi |
| Ir_{0.06}Ti_{0.98}Nb_{1.96}O₇ | 20.37888 | 3.800701 | 11.91216 | 796.1592843 | Single |
| Pt_{0.06}Ti_{0.98}Nb_{1.96}O₇ | 20.36817 | 3.798939 | 11.91028 | 795.3734921 | Multi |
| Au_{0.06}Ti_{0.98}Nb_{1.96}O₇ | 20.36499 | 3.798718 | 11.9077 | 795.0972351 | Single |
| Pb_{0.06}Ti_{0.98}Nb_{1.96}O₇ | 20.36626 | 3.797692 | 11.90285 | 794.8385485 | Multi |
| TiNb₂O₇ | 20.37518 | 3.800141 | 11.91573 | 796.1745566 | Single |

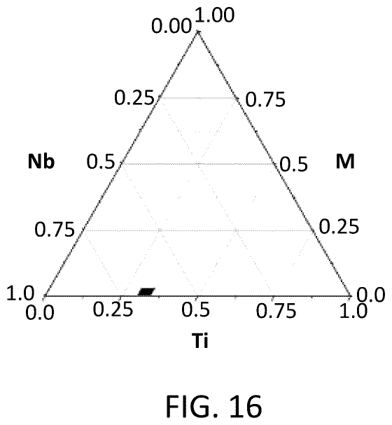

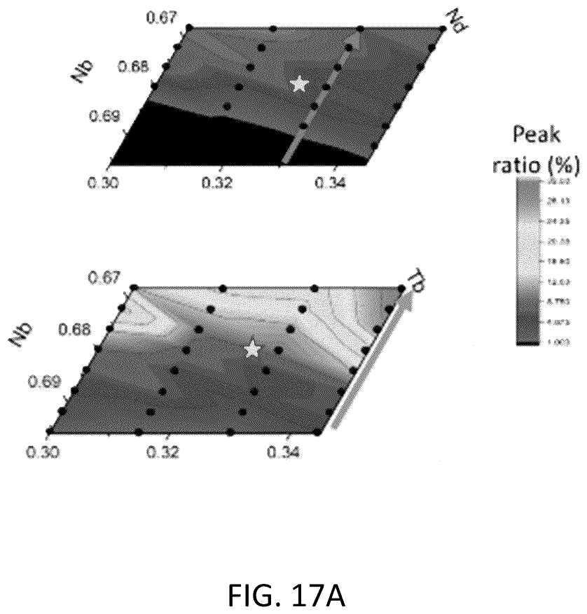

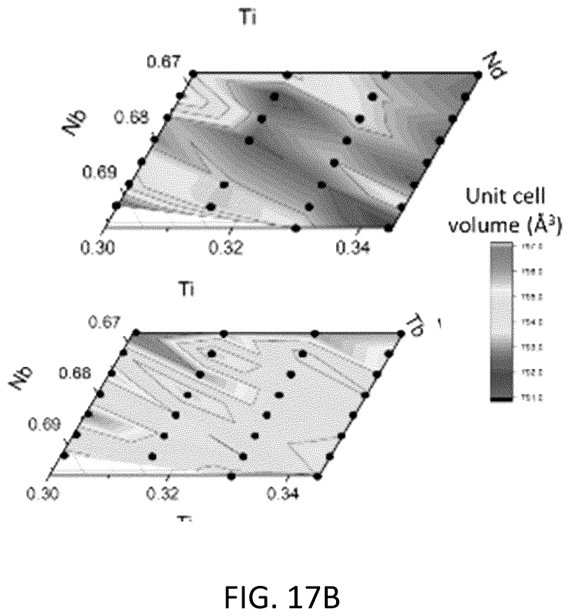

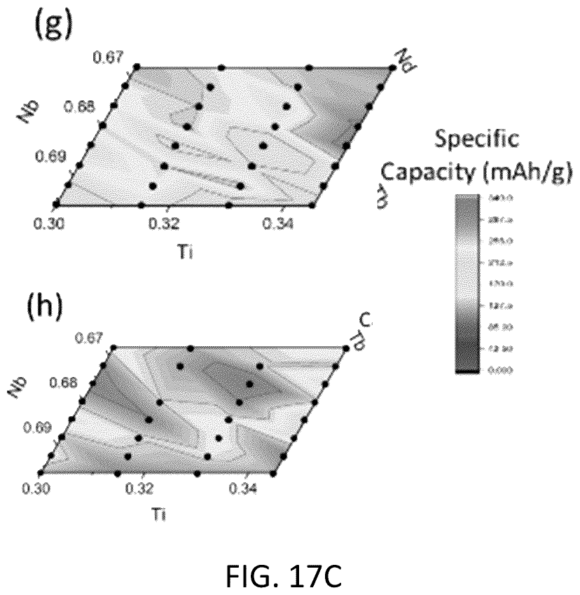

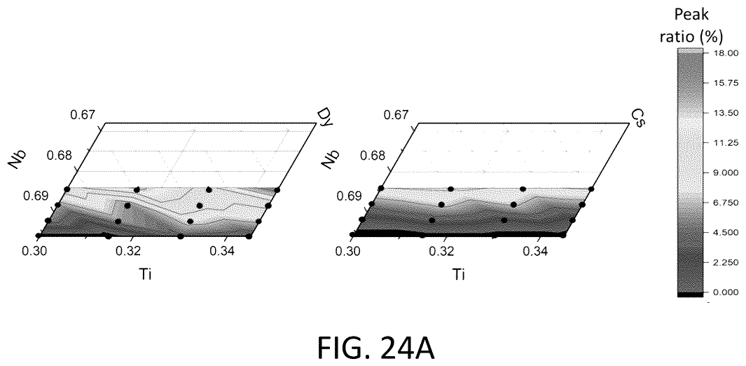

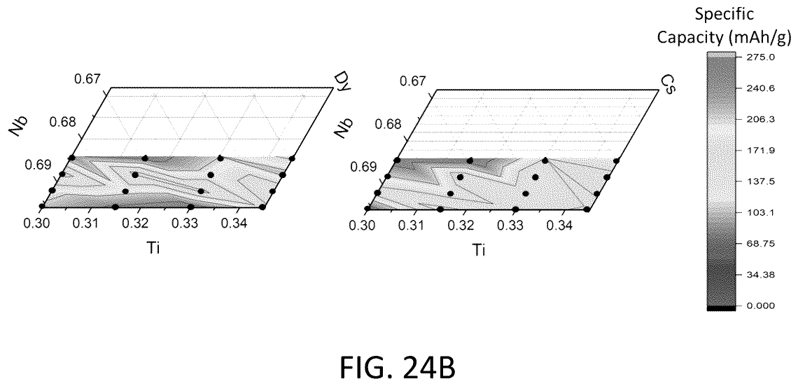

Given that some of the best electrochemically performing materials did not exhibit a single-phase, additional samples of TNO doped with either Nd, Tb, Cs, or Dy were synthesized and examined to see if single phase material exist at lower doping levels (Table 4). The compositions tested in this extended compositional analysis represent a small section of the Ti—Nb-M-O Gibbs' triangle as shown in FIG. 16. FIGS. 17A and 17B show the results of the expanded compositional analysis screen for Tb and Nd doped TNO. Table 4 shows the results for all of the additional doped TNO materials tested. A single-phase region was found in the Nd space only (FIG. 17A), while the entire Tb space was found to be multi-phase (FIG. 17A). The secondary phases are thought to be the respective lanthanide oxides that form with the doped atoms. The growth of these secondary phases can be seen in the growth of a PXRD peak around 29.5° (FIGS. 15A and 15B). The height of this secondary peak as a percentage of the largest TNO peak is given in FIG. 17A, which shows an increase correlated to the amount of dopant added. The relative peak ratio is much higher for Tb-doped TNO samples, which at the highest dopant concentrations shows secondary phase peak ratios of 20% to 30%. This suggests co-existence to a nearby material on the Tb—Ti—O pseudobinary. Nd substituted samples showed less secondary phase at all compositions, with a maximum of 8% relative peak ratio found at the highest dopant level indicating co-existence to a material further up in the Gibbs' triangle, perhaps even at the Nd corner. By analyzing the TNO unit cell parameters via Pawley fits, it can be seen that beyond the single-phase regions of both compositional spaces, the unit cell volumes do not change considerably with a change in dopant concentration (FIG. 17B), again consistent with coexistence. This lack of volume growth implies that TNO reaches a saturation point as dopant is added, after which all remaining dopant atoms form secondary phases, explaining the growth in these phases. It can be seen in the regions closest to undoped TNO that the unit cell volumes are very close to undoped TNO volumes. It was additionally seen in the multi-phase regions that there was not a large variation in the specific discharge capacity (see Table 7, discussed herein), implying that these secondary phases are not electrochemically active in the relevant voltage window. Related plots for an expanded compositional space of Dy-TNO and Cs-TNO are shown in FIGS. 24A and 24B. As in the case of Nd, it appears that Dy and Cs saturate in TNO at low levels. The implication is that very small doping levels may be sufficient to obtain the benefits in electrochemistry.

TABLE 4
Expanded Nd, Tb, Cs, and Dy doped TNO PXRD properties

| | | | | | Single |
|---|---|---|---|---|---|
| | | | | Unit cell | or Multi |
| Composition | Cell a [A] | Cell b [A] | Cell c [A] | volume[A³] | Phase |
| Nd_{0.012}Ti_{0.9}Nb_{2.088}O₇ | 20.3883 | 3.803191 | 11.92249 | 797.0428 | Single |
| Nd_{0.012}Ti_{0.945}Nb_{2.043}O₇ | 20.39309 | 3.803099 | 11.91426 | 796.3136 | Single |
| Nd_{0.012}Ti_{0.99}Nb_{1.998}O₇ | 20.372 | 3.7961 | 11.89756 | 793.6373 | Single |
| Nd_{0.012}Ti_{1.035}Nb_{1.953}O₇ | 20.34325 | 3.791856 | 11.88865 | 791.6732 | Single |
| Nd_{0.024}Ti_{0.9}Nb_{2.076}O₇ | 20.44216 | 3.800294 | 11.91084 | 796.9669 | Single |
| Nd_{0.024}Ti_{0.945}Nb_{2.031}O₇ | 20.35763 | 3.794833 | 11.89819 | 793.3234 | Single |
| Nd_{0.024}Ti_{0.99}Nb_{1.986}O₇ | 20.36151 | 3.794985 | 11.89413 | 792.9921 | Single |
| Nd_{0.024}Ti_{1.035}Nb_{1.941}O₇ | 20.37549 | 3.796387 | 11.89627 | 793.7579 | Multi |
| Nd_{0.036}Ti_{0.9}Nb_{2.064}O₇ | 20.41023 | 3.796902 | 11.90398 | 794.1347 | Single |
| Nd_{0.036}Ti_{0.945}Nb_{2.019}O₇ | 20.38537 | 3.796051 | 11.89371 | 793.3454 | Single |
| Nd_{0.036}Ti_{0.99}Nb_{1.974}O₇ | 20.36316 | 3.793803 | 11.89108 | 792.5686 | Multi |
| Nd_{0.036}Ti_{1.035}Nb_{1.929}O₇ | 20.34574 | 3.793778 | 11.89119 | 792.418 | Multi |
| Nd_{0.048}Ti_{0.9}Nb_{2.052}O₇ | 20.37018 | 3.796794 | 11.89373 | 793.8358 | Single |
| Nd_{0.048}Ti_{0.945}Nb_{2.007}O₇ | 18.36742 | 3.631853 | 13.40763 | 749.0861 | Multi |
| Nd_{0.048}Ti_{0.99}Nb_{1.962}O₇ | 20.35474 | 3.795468 | 11.89508 | 793.2682 | Multi |
| Nd_{0.048}Ti_{1.035}Nb_{1.917}O₇ | 20.35079 | 3.794505 | 11.89324 | 792.9084 | Multi |
| Nd_{0.06}Ti_{0.9}Nb_{2.04}O₇ | 20.37806 | 3.794811 | 11.8928 | 792.9046 | Multi |
| Nd_{0.06}Ti_{0.945}Nb_{1.995}O₇ | 20.35474 | 3.793632 | 11.89083 | 792.4418 | Multi |
| Nd_{0.06}Ti_{0.99}Nb_{1.95}O₇ | 20.35237 | 3.795017 | 11.89169 | 792.9043 | Multi |
| Nd_{0.06}Ti_{1.035}Nb_{1.905}O₇ | 20.35149 | 3.794145 | 11.89133 | 792.5951 | Multi |
| Nd_{0.072}Ti_{0.9}Nb_{2.028}O₇ | 20.36086 | 3.794739 | 11.89433 | 793.1951 | Multi |
| Nd_{0.072}Ti_{0.945}Nb_{1.983}O₇ | 20.3549 | 3.794647 | 11.89078 | 792.7834 | Multi |
| Nd_{0.072}Ti_{0.99}Nb_{1.938}O₇ | 20.35538 | 3.796857 | 11.89221 | 793.5338 | Multi |
| Nd_{0.072}Ti_{1.035}Nb_{1.893}O₇ | 20.34991 | 3.794931 | 11.88961 | 792.9 | Multi |
| Nd_{0.084}Ti_{0.9}Nb_{2.016}O₇ | 20.42545 | 3.799274 | 11.90093 | 795.6993 | Multi |
| Nd_{0.084}Ti_{0.945}Nb_{1.971}O₇ | 20.34861 | 3.793222 | 11.88995 | 792.2556 | Multi |
| Nd_{0.084}Ti_{0.99}Nb_{1.926}O₇ | 20.35276 | 3.796092 | 11.89177 | 793.3612 | Multi |
| Nd_{0.084}Ti_{1.035}Nb_{1.881}O₇ | 20.34861 | 3.794268 | 11.88998 | 792.627 | Multi |
| Nd_{0.096}Ti_{0.9}Nb_{2.004}O₇ | 20.37238 | 3.79637 | 11.89534 | 793.8621 | Multi |
| Nd_{0.096}Ti_{0.945}Nb_{1.959}O₇ | 20.36079 | 3.797392 | 11.8966 | 794.0988 | Multi |
| Nd_{0.096}Ti_{0.99}Nb_{1.914}O₇ | 20.35386 | 3.794672 | 11.89154 | 793.0655 | Multi |
| Nd_{0.096}Ti_{1.035}Nb_{1.869}O₇ | 20.35014 | 3.793572 | 11.8895 | 792.7084 | Multi |
| Tb_{0.012}Ti_{0.9}Nb_{2.088}O₇ | 20.36583 | 3.800323 | 11.91502 | 795.6076 | Multi |
| Tb_{0.012}Ti_{0.945}Nb_{2.043}O₇ | 20.36179 | 3.797839 | 11.90834 | 794.703 | Multi |
| Tb_{0.012}Ti_{0.99}Nb_{1.998}O₇ | 20.35976 | 3.796299 | 11.89668 | 793.7061 | Multi |
| Tb_{0.012}Ti_{1.035}Nb_{1.953}O₇ | 20.35766 | 3.797932 | 11.89488 | 793.9706 | Multi |
| Tb_{0.024}Ti_{0.9}Nb_{2.076}O₇ | 20.38661 | 3.79958 | 11.90165 | 795.2157 | Multi |
| Tb_{0.024}Ti_{0.945}Nb_{2.031}O₇ | 20.3708 | 3.797961 | 11.90017 | 794.5076 | Multi |
| Tb_{0.024}Ti_{0.99}Nb_{1.986}O₇ | 20.37077 | 3.799525 | 11.90058 | 795.0046 | Multi |
| Tb_{0.024}Ti_{1.035}Nb_{1.941}O₇ | 20.36073 | 3.79515 | 11.90011 | 793.6353 | Multi |
| Tb_{0.036}Ti_{0.9}Nb_{2.064}O₇ | 20.37159 | 3.798656 | 11.89408 | 794.2793 | Multi |
| Tb_{0.036}Ti_{0.945}Nb_{2.019}O₇ | 20.39756 | 3.798371 | 11.90158 | 794.9211 | Multi |
| Tb_{0.036}Ti_{0.99}Nb_{1.974}O₇ | 20.36844 | 3.79762 | 11.89894 | 794.2813 | Multi |
| Tb_{0.036}Ti_{1.035}Nb_{1.929}O₇ | 20.36375 | 3.797355 | 11.89667 | 794.038 | Multi |
| Tb_{0.048}Ti_{0.9}Nb_{2.052}O₇ | 20.40892 | 3.800772 | 11.90515 | 796.1258 | Multi |
| Tb_{0.048}Ti_{0.945}Nb_{2.007}O₇ | 20.3822 | 3.798768 | 11.90086 | 794.9955 | Multi |
| Tb_{0.048}Ti_{0.99}Nb_{1.962}O₇ | 20.36789 | 3.79921 | 11.89974 | 794.7742 | Multi |
| Tb_{0.048}Ti_{1.035}Nb_{1.917}O₇ | 20.36822 | 3.796363 | 11.90465 | 794.4393 | Multi |
| Tb_{0.06}Ti_{0.9}Nb_{2.04}O₇ | 20.37425 | 3.796928 | 11.8979 | 793.8997 | Multi |
| Tb_{0.06}Ti_{0.945}Nb_{1.995}O₇ | 20.36037 | 3.795887 | 11.89316 | 793.3 | Multi |
| Tb_{0.06}Ti_{0.99}Nb_{1.95}O₇ | 20.37341 | 3.798521 | 11.90067 | 794.7189 | Multi |
| Tb_{0.06}Ti_{1.035}Nb_{1.905}O₇ | 20.3613 | 3.796775 | 11.89787 | 793.925 | Multi |
| Tb_{0.072}Ti_{0.9}Nb_{2.028}O₇ | 20.37998 | 3.800533 | 11.90382 | 795.5468 | Multi |
| Tb_{0.072}Ti_{0.945}Nb_{1.983}O₇ | 20.3957 | 3.800892 | 11.90803 | 795.9225 | Multi |
| Tb_{0.072}Ti_{0.99}Nb_{1.938}O₇ | 20.3631 | 3.798674 | 11.89652 | 794.4201 | Multi |
| Tb_{0.072}Ti_{1.035}Nb_{1.893}O₇ | 20.37364 | 3.798488 | 11.90271 | 795.1216 | Multi |
| Tb_{0.084}Ti_{0.9}Nb_{2.016}O₇ | 20.35829 | 3.797866 | 11.89724 | 794.1462 | Multi |
| Tb_{0.084}Ti_{0.945}Nb_{1.971}O₇ | 20.35228 | 3.79635 | 11.89223 | 793.2671 | Multi |
| Tb_{0.084}Ti_{0.99}Nb_{1.926}O₇ | 20.35621 | 3.797004 | 11.89557 | 793.7693 | Multi |
| Tb_{0.084}Ti_{1.035}Nb_{1.881}O₇ | 20.37242 | 3.799429 | 11.90154 | 795.0232 | Multi |
| Tb_{0.096}Ti_{0.9}Nb_{2.004}O₇ | 20.44152 | 3.80124 | 11.91346 | 797.0999 | Multi |
| Tb_{0.096}Ti_{0.945}Nb_{1.959}O₇ | 20.37913 | 3.800256 | 11.90598 | 795.7635 | Multi |
| Tb_{0.096}Ti_{0.99}Nb_{1.914}O₇ | 20.3807 | 3.799849 | 11.91165 | 796.0453 | Multi |
| Tb_{0.096}Ti_{1.035}Nb_{1.869}O₇ | 20.38095 | 3.799233 | 11.90988 | 795.8666 | Multi |
| Cs_{0.012}Ti_{0.9}Nb_{2.088}O₇ | 20.44225 | 3.806481 | 11.93251 | 798.514 | Single |
| Cs_{0.012}Ti_{0.945}Nb_{2.043}O₇ | 20.3804 | 3.803363 | 11.91758 | 795.8341 | Single |
| Cs_{0.012}Ti_{0.99}Nb_{1.998}O₇ | 20.35888 | 3.800106 | 11.91235 | 794.9229 | Single |
| Cs_{0.012}Ti_{1.035}Nb_{1.953}O₇ | 20.36567 | 3.803051 | 11.91727 | 795.74 | Single |
| Cs_{0.024}Ti_{0.9}Nb_{2.076}O₇ | 20.42286 | 3.805707 | 11.92395 | 797.8681 | Multi |
| Cs_{0.024}Ti_{0.945}Nb_{2.031}O₇ | 20.36703 | 3.800846 | 11.90324 | 794.7548 | Multi |
| Cs_{0.024}Ti_{0.99}Nb_{1.986}O₇ | 20.36598 | 3.801929 | 11.90787 | 795.2122 | Multi |
| Cs_{0.024}Ti_{1.035}Nb_{1.941}O₇ | 20.35926 | 3.799213 | 11.90391 | 794.6252 | Multi |
| Cs_{0.036}Ti_{0.9}Nb_{2.064}O₇ | 20.38025 | 3.802068 | 11.90757 | 795.4808 | Multi |
| CS_{0.036}Ti_{0.945}Nb_{2.019}O₇ | 20.36494 | 3.800797 | 11.91076 | 794.9431 | Multi |
| Cs_{0.036}Ti_{0.99}Nb_{1.974}O₇ | 20.35485 | 3.798875 | 11.89966 | 794.065 | Multi |
| Cs_{0.036}Ti_{1.035}Nb_{1.929}O₇ | 20.36311 | 3.800123 | 11.90924 | 794.9428 | Multi |
| Cs_{0.048}Ti_{0.9}Nb_{2.052}O₇ | 20.38777 | 3.802843 | 11.90768 | 795.8057 | Multi |
| Cs_{0.048}Ti_{0.945}Nb_{2.007}O₇ | 20.36435 | 3.80187 | 11.91622 | 795.5195 | Multi |
| Cs_{0.048}Ti_{0.99}Nb_{1.962}O₇ | 20.36204 | 3.800163 | 11.9051 | 794.7689 | Multi |
| Cs_{0.048}Ti_{1.035}Nb_{1.917}O₇ | 20.36625 | 3.799286 | 11.90385 | 794.687 | Multi |
| Dy_{0.012}Ti_{0.9}Nb_{2.088}O₇ | 20.38366 | 3.802943 | 11.91827 | 796.5316 | Single |
| Dy_{0.012}Ti_{0.945}Nb_{2.043}O₇ | 20.47481 | 3.810752 | 11.93591 | 801.0133 | Single |
| Dy_{0.012}Ti_{0.99}Nb_{1.998}O₇ | 20.37328 | 3.800288 | 11.92193 | 796.1721 | Multi |
| Dy_{0.012}Ti_{1.035}Nb_{1.953}O₇ | 20.37239 | 3.800191 | 11.91789 | 796.1294 | Multi |
| Dy_{0.024}Ti_{0.9}Nb_{2.076}O₇ | 20.44261 | 3.808978 | 11.9368 | 800.0341 | Multi |
| Dy_{0.024}Ti_{0.945}Nb_{2.031}O₇ | 20.38197 | 3.804423 | 11.92784 | 797.469 | Multi |
| Dy_{0.024}Ti_{0.99}Nb_{1.986}O₇ | 20.37246 | 3.800632 | 11.91279 | 795.9621 | Multi |
| Dy_{0.024}Ti_{1.035}Nb_{1.941}O₇ | 20.3798 | 3.800952 | 11.92446 | 796.6114 | Multi |
| Dy_{0.036}Ti_{0.9}Nb_{2.064}O₇ | 20.38568 | 3.802645 | 11.91965 | 796.5566 | Multi |
| Dy_{0.036}Ti_{0.945}Nb_{2.019}O₇ | 20.41952 | 3.804709 | 11.92494 | 797.9263 | Multi |
| Dy_{0.036}Ti_{0.99}Nb_{1.974}O₇ | 20.37455 | 3.802315 | 11.92784 | 796.7448 | Multi |
| Dy_{0.036}Ti_{1.035}Nb_{1.929}O₇ | 20.38715 | 3.805171 | 11.94219 | 798.5797 | Multi |
| Dy_{0.048}Ti_{0.9}Nb_{2.052}O₇ | 20.45153 | 3.808583 | 11.94128 | 800.3503 | Multi |
| Dy_{0.048}Ti_{0.945}Nb_{2.007}O₇ | 20.48095 | 3.808383 | 11.96121 | 802.0266 | Multi |
| Dy_{0.048}Ti_{0.99}Nb_{1.962}O₇ | 20.38001 | 3.803666 | 11.9237 | 797.2534 | Multi |
| Dy_{0.048}Ti_{1.035}Nb_{1.917}O₇ | 20.39577 | 3.80407 | 11.9345 | 798.6063 | Multi |

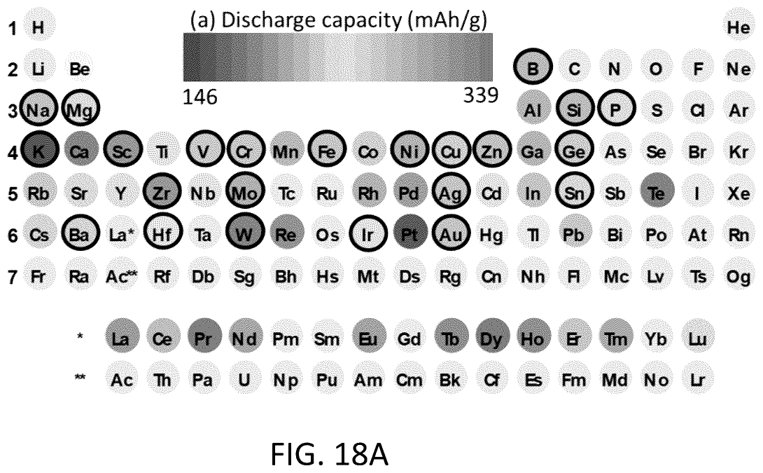

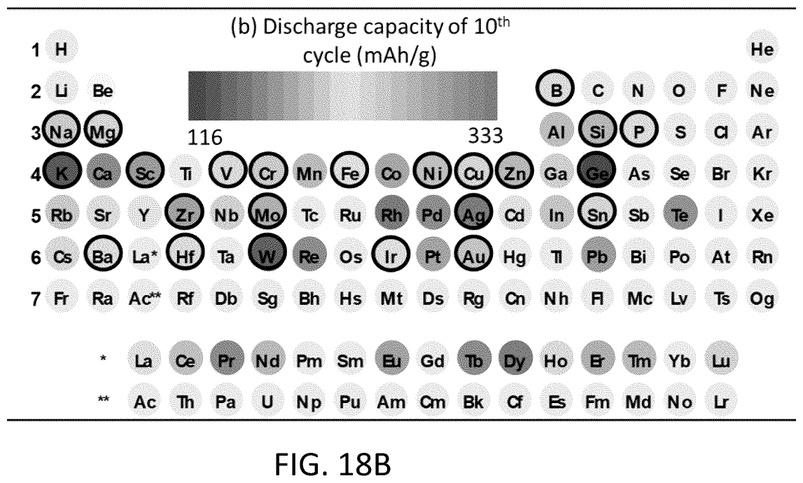

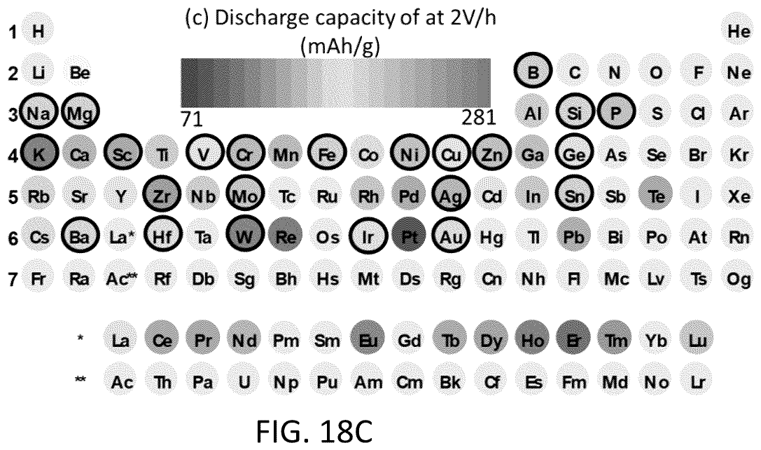

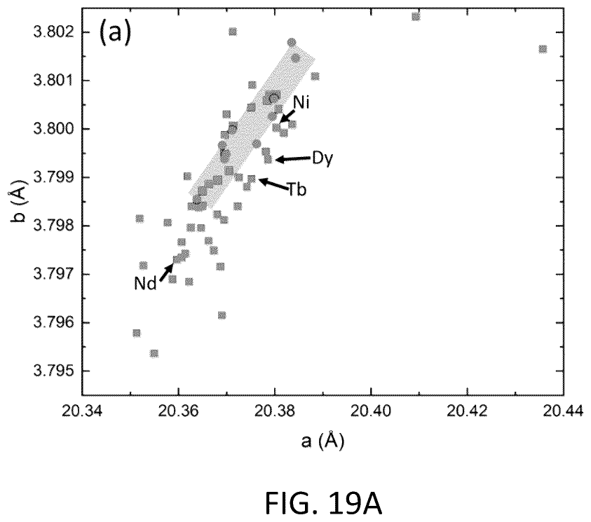

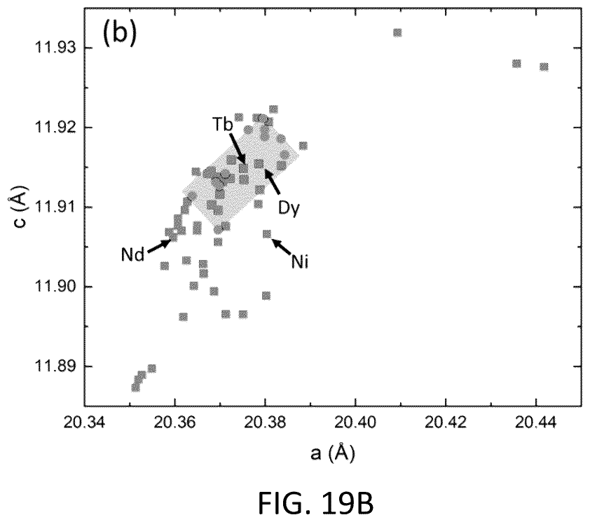

In cases where coexistence is found (i.e., the material is multiphase), especially in the systematic screening at 2 atom-% doping level, it may be beneficial to determine if some of the 2 atom-% dopant integrated into the structure. Previous work on the Nb—Ti—O pseudobinary shows that no solid solution exists around TNO. This means that changes in lattice parameters can only occur if some dopants integrate into TNO (i.e., a TNO with a different Ti:Nb content cannot be formed). Thus, to determine whether the samples were being substituted at some level below 2 atom-%, the lattice parameters of all materials were analyzed (FIGS. 19A and 19B). There is a weakly linear correlation between a against both b and c lattice parameters. Thus, it is implied that the growth of one lattice parameter is linked to the growth of others. The lattice parameters of undoped samples were also plotted in FIGS. 18A and 18B and they show some small variation with all of them contained within the marked rectangles. Data points outside of these regions cannot be achieved without dopants being integrated in the structure. Careful analysis shows that only five dopants lie outside of both undoped regions in FIGS. 18A and 18B: Pt, Eu, Re, Te, and Zn. It should also be noted that these five samples may have also substituted in some amount; however, the lattice parameter shifts were insufficient to be observed. In sum, 47 different dopants were integrated into the TNO structure.

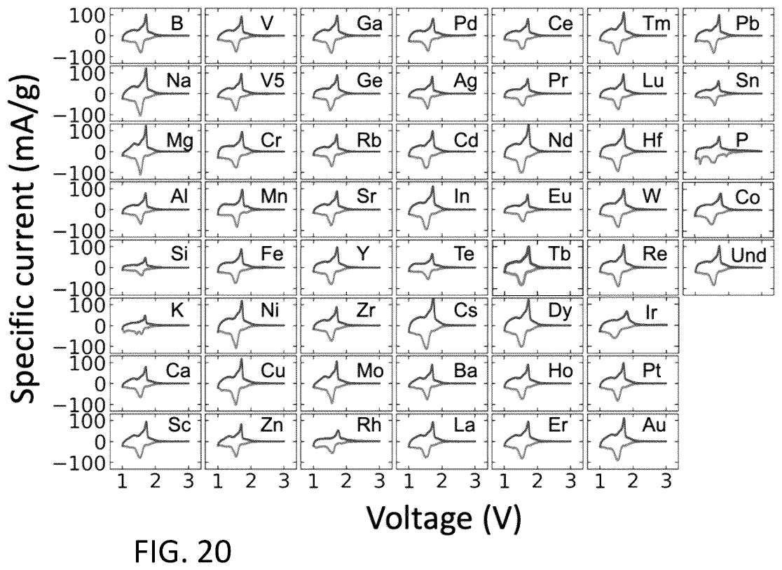

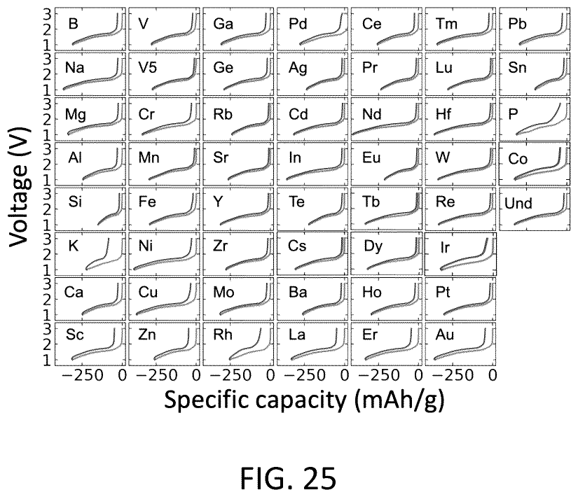

Extensive electrochemical measurements were carried out on all Example 2 samples in the form of cyclic voltammetry (CV) between the voltages of 1 V and 3 V vs Li (FIG. 20). Materials were cycled at five different rates: 0.1 V per h, 0.2 V per h, 0.5 V per h, 1 V per h and 2 V per h. After the rate testing, the materials were then cycled at 0.1 V per h for 10 full cycles (400 hours or about 17 days of cycling). Cells were run at both room temperature and 37° C. All CVs show the same general shape, characteristic of TNO (FIG. 20, Und is undoped). These CVs show a strong charge peak at about 1.55 V and a discharge peak at 1.75 V, which has previously been attributed to the Nb⁵⁺/Nb³⁺ redox couples. Very little electrochemical activity is seen between 2.2 V and 3 V. Voltage curves for all dopants were also calculated from these CVs as shown in FIG. 25 and generally show small overpotentials and high reversibility.

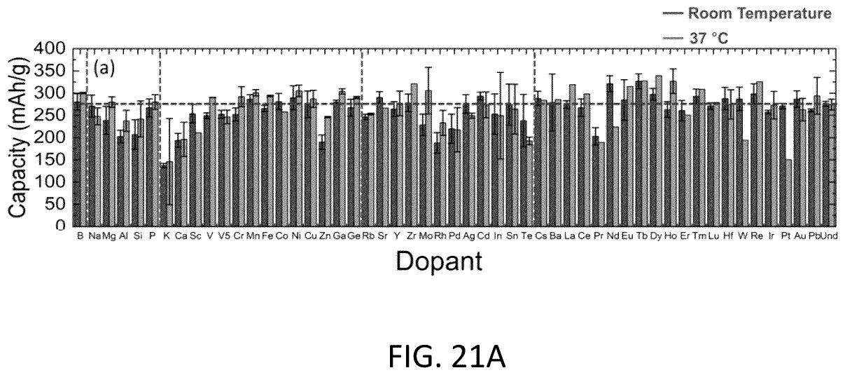

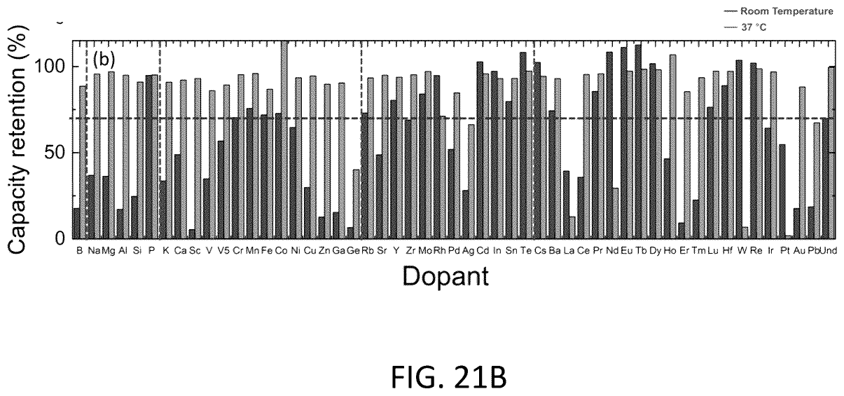

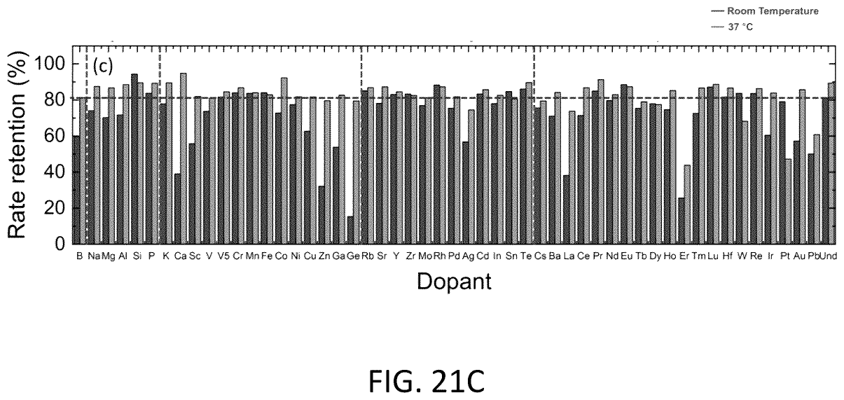

From the Example 2 CVs, a host of battery performance parameters were calculated as seen in FIG. 18A, 18B, 18C, 21A, 21B, 21C, 22, 23, and Table 5 and Table 6. Some performance parameters were calculated for the expanded set of doped TNO (Table 7). Many materials featured high discharge capacities at 0.1 V per hour at both room temperature and 37° C., with several doped TNO showing capacities above 320 mAh g⁻¹ (FIG. 21A and Table 5). In total, 19 dopants show improved capacities over the undoped (above the error on the mean shown by the error bars on FIG. 21A). Of particular interest were several lanthanide containing materials such as Nd-TNO which performed well at room temperature, Dy-TNO which performed well at 37° C., and Tb-TNO which displayed strong capacities at both temperatures (see FIG. 21A and Table 5). Additionally, several transition metal containing materials such as Mn-TNO, Ni-TNO and Mo-TNO also outperformed undoped TNO. Undoped TNO showed a strong discharge capacity of around 275 mAh g⁻¹ at both room temperature and 37° C.

Some of the high-performance doped materials of Example 2 also show strong performances in capacity retention over extended cycling periods. Capacity retention was measured by comparing the first discharge capacity to the tenth discharge capacity (FIG. 21B and Table 6). Performance over extended cycling may be advantageous in materials used in implantable devices, as their replacement can often be invasive to patients. Thus, it may be advantageous for a material used in an implantable device to have a high-capacity retention at 37° C.

Capacity retentions were found to be much more variable at room temperature, while nearly all doped TNO samples showed strong performance at 37° C. At room temperature, several of the lanthanide dopants had a capacity retention over 100%, implying a small growth in capacity over extended cycling. At this temperature, several transition metals outperformed the undoped TNO capacity retention of 70%. At 37° C. however, a majority of substituted-TNO materials had capacity retentions of over 90%. Several of the lanthanide doped materials such as Tb-TNO and Dy-TNO showed retentions of over 98% at the elevated temperature. Several transition metals also showed strong performance at this temperature. For example, Mo doped TNO had a capacity retention of 97%. Some of this perceived improvement in performance may be attributed to the combinatorial cell cycling better at higher temperatures due to a softening of the polymer seal. However, this would affect the entire cell, and theoretically affect the samples at the edges most strongly, which is not seen in the data at room temperature. Therefore, the majority of this retention performance is attributed to the materials.

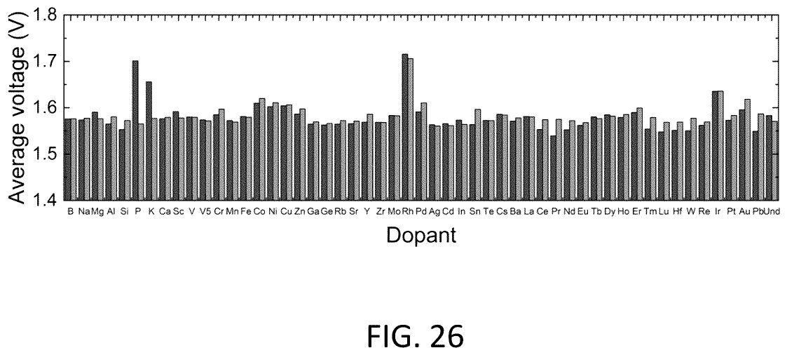

FIG. 21C and Table 6 show the rate retention of the doped-TNO samples at both room temperature and 37° C. The rate retention was calculated by dividing the discharge capacity at a rate of 2 V h⁻¹ by the initial discharge capacity at 0.1 V h⁻¹. Performance was similar between the two temperatures tested, with many materials showing rate retentions over 80% when cycled at over an order of magnitude increase in rate. For many of these doped-TNO materials, this means capacities of over 230 mAh g⁻¹ for a discharge taking only one hour (though nearly all the capacity is achieved in a 30 min window). Average voltages of all dopants were also calculated showing relatively low variation, with most materials showing average voltages around 1.56 (FIG. 26 and Table 6).

The discharge capacity (FIG. 18A), discharge capacity after ten cycles (FIG. 18B), and the discharge capacity at 2V/h (FIG. 18C), determined at 37° C. are plotted on the periodic table and show that much of the periodic table is of interest for doped TNO materials. For example, the Re doped sample showed particularly high capacities at the higher sweep rates, outperforming all other doped TNO samples at 2 V/h despite not being one of the leaders at the slower rates. This suggests opportunities to benefit from co-doping, where a small amount of Re may be beneficial for rate performance while adding another dopant, like Nd, in even smaller quantities to help with the low rate capacities. It is also of interest that Re was so beneficial despite very little changes in the lattice parameters as discussed above. In fact it was impossible to conclude whether or not any dopant was integrated based on lattice parameters.

TABLE 5
Discharge capacity and average voltage of doped and undoped TNO materials

| | | | Number | | | | |
|---|---|---|---|---|---|---|---|
| | RT | Error | of samples | 37° C. | Error for | RT | 37° C. |
| | discharge | for RT | for RT | discharge | 37° C. | average | average |
| | capacity | discharge | discharge | capacity | discharge | voltage | voltage |
| Sample | (mAh/g) | capacity | capacities | (mAh/g) | capacity | (V) | (V) |
| B_{0.06}Ti_{0.98}Nb_{1.96}O₇ | 280.2404 | 18.45288 | 5 | 300.3428 | 1.56783 | 1.57607 | 1.57628 |
| Na_{0.06}Ti_{0.98}Nb_{1.96}O₇ | 270.9676 | 24.66806 | 5 | 247.7863 | 19.06016 | 1.57388 | 1.57782 |
| Mg_{0.06}Ti_{0.98}Nb_{1.96}O₇ | 238.5905 | 31.42299 | 5 | 279.7786 | 11.92188 | 1.5906 | 1.57659 |
| Al_{0.06}Ti_{0.98}Nb_{1.96}O₇ | 202.3194 | 14.33008 | 5 | 237.727 | 23.8365 | 1.565 | 1.58093 |
| Si_{0.06}Ti_{0.98}Nb_{1.96}O₇ | 207.0021 | 33.45453 | 5 | 241.8389 | 40.53201 | 1.55307 | 1.57245 |
| P_{0.06}Ti_{0.98}Nb_{1.96}O₇ | 267.0038 | 20.18052 | 5 | 280.0783 | 16.57547 | 1.70146 | 1.56526 |
| K_{0.06}Ti_{0.98}Nb_{1.96}O₇ | 136.9643 | 4.83782 | 2 | 145.978 | 97.03003 | 1.65605 | 1.57721 |
| Ca_{0.06}Ti_{0.98}Nb_{1.96}O₇ | 193.9467 | 15.78232 | 3 | 196.1417 | 38.58135 | 1.57638 | 1.57929 |
| Sc_{0.06}Ti_{0.98}Nb_{1.96}O₇ | 253.7492 | 21.02262 | 4 | 211.2873 | | 1.59173 | 1.57803 |
| V_{0.06}Ti_{0.98}Nb_{1.96}O₇ | 249.3474 | 6.54437 | 5 | 290.4546 | 0.24726 | 1.58041 | 1.5796 |
| V_{0.06}Ti_{0.98}Nb_{1.96}O₇ | 252.601 | 9.00914 | 5 | 246.5314 | 15.32969 | 1.57401 | 1.57177 |
| Cr_{0.06}Ti_{0.98}Nb_{1.96}O₇ | 252.0901 | 14.86383 | 5 | 292.0429 | 22.37939 | 1.58505 | 1.59705 |
| Mn_{0.06}Ti_{0.98}Nb_{1.96}O₇ | 286.2781 | 10.55317 | 5 | 300.9115 | 7.10656 | 1.57252 | 1.56931 |
| Fe_{0.06}Ti_{0.98}Nb_{1.96}O₇ | 265.5972 | 6.78113 | 5 | 293.6386 | 1.47747 | 1.58133 | 1.57952 |
| Co_{0.06}Ti_{0.98}Nb_{1.96}O₇ | 281.0718 | 18.30592 | 4 | 257.992 | | 1.61 | 1.6203 |
| Ni_{0.06}Ti_{0.98}Nb_{1.96}O₇ | 289.5631 | 27.0795 | 4 | 305.2718 | 12.9579 | 1.60234 | 1.6112 |
| Cu_{0.06}Ti_{0.98}Nb_{1.96}O₇ | 274.7316 | 29.9732 | 4 | 287.1459 | 19.25553 | 1.60448 | 1.60635 |
| Zn_{0.06}Ti_{0.98}Nb_{1.96}O₇ | 189.8436 | 16.29326 | 4 | 246.1302 | 1.30491 | 1.58637 | 1.59756 |
| Ga_{0.06}Ti_{0.98}Nb_{1.96}O₇ | 279.8358 | 4.26077 | 5 | 303.9391 | 6.18791 | 1.56478 | 1.56967 |
| Ge_{0.06}Ti_{0.98}Nb_{1.96}O₇ | 267.2292 | 18.92295 | 5 | 290.1375 | 2.57973 | 1.56289 | 1.56631 |
| Rb_{0.06}Ti_{0.98}Nb_{1.96}O₇ | 246.4989 | 6.10028 | 5 | 252.8757 | 1.81681 | 1.56479 | 1.57263 |
| Sr_{0.06}Ti_{0.98}Nb_{1.96}O₇ | 290.486 | 13.0205 | 4 | 266.6094 | | 1.56576 | 1.57139 |
| Y_{0.06}Ti_{0.98}Nb_{1.96}O₇ | 264.1313 | 16.83379 | 5 | 276.8417 | 27.63605 | 1.56914 | 1.58615 |
| Zr_{0.06}Ti_{0.98}Nb_{1.96}O₇ | 278.1826 | 19.70281 | 5 | 321.468 | | 1.56876 | 1.56853 |
| Mo_{0.06}Ti_{0.98}Nb_{1.96}O₇ | 228.8099 | 24.05402 | 5 | 305.6256 | 52.57011 | 1.58364 | 1.58269 |
| Rh_{0.06}Ti_{0.98}Nb_{1.96}O₇ | 188.2154 | 22.9556 | 4 | 233.1455 | 28.18694 | 1.71571 | 1.70599 |
| Pd_{0.06}Ti_{0.98}Nb_{1.96}O₇ | 219.9917 | 32.81673 | 5 | 217.4883 | 49.75353 | 1.59113 | 1.61057 |
| Ag_{0.06}Ti_{0.98}Nb_{1.96}O₇ | 275.6222 | 21.13813 | 5 | 249.2708 | 5.84195 | 1.56352 | 1.56072 |
| Cd_{0.06}Ti_{0.98}Nb_{1.96}O₇ | 293.4921 | 8.94148 | 5 | 273.6567 | 29.03507 | 1.56597 | 1.56142 |
| In_{0.06}Ti_{0.98}Nb_{1.96}O₇ | 252.9928 | 45.43726 | 5 | 249.3233 | 97.36275 | 1.57358 | 1.56415 |
| Sn_{0.06}Ti_{0.98}Nb_{1.96}O₇ | 274.2976 | 46.2148 | 5 | 264.0196 | 56.00566 | 1.56388 | 1.59652 |
| Te_{0.06}Ti_{0.98}Nb_{1.96}O₇ | 238.261 | 59.01232 | 5 | 192.693 | 8.49996 | 1.57269 | 1.57226 |
| Cs_{0.06}Ti_{0.98}Nb_{1.96}O₇ | 288.2815 | 15.90203 | 5 | 284.139 | | 1.58619 | 1.58458 |
| Ba_{0.06}Ti_{0.98}Nb_{1.96}O₇ | 278.8228 | 64.23488 | 4 | 285.6329 | | 1.57109 | 1.57824 |
| La_{0.06}Ti_{0.98}Nb_{1.96}O₇ | 274.4057 | 8.50576 | 2 | 319.5962 | | 1.58135 | 1.58054 |
| Ce_{0.06}Ti_{0.98}Nb_{1.96}O₇ | 267.3069 | 19.85423 | 3 | 298.3674 | | 1.55309 | 1.57462 |
| Pr_{0.06}Ti_{0.98}Nb_{1.96}O₇ | 202.975 | 19.52972 | 3 | 189.3232 | | 1.53944 | 1.57512 |
| Nd_{0.06}Ti_{0.98}Nb_{1.96}O₇ | 321.1253 | 18.04584 | 4 | | | 1.55244 | 1.5722 |
| Eu_{0.06}Ti_{0.98}Nb_{1.96}O₇ | 284.5504 | 45.66673 | 3 | 315.0756 | | 1.56226 | 1.5679 |
| Tb_{0.06}Ti_{0.98}Nb_{1.96}O₇ | 326.7144 | 16.61222 | 3 | 327.9263 | | 1.58063 | 1.5766 |
| Dy_{0.06}Ti_{0.98}Nb_{1.96}O₇ | 297.3591 | 13.08209 | 4 | 339.455 | | 1.58511 | 1.58194 |
| Ho_{0.06}Ti_{0.98}Nb_{1.96}O₇ | 263.1953 | 17.7256 | 4 | 326.7811 | 27.53973 | 1.57907 | 1.58548 |
| Er_{0.06}Ti_{0.98}Nb_{1.96}O₇ | 260.8576 | 22.91518 | 3 | 250.8176 | | 1.58996 | 1.5997 |
| Tm_{0.06}Ti_{0.98}Nb_{1.96}O₇ | 292.7216 | 16.54279 | 4 | 309.0318 | | 1.55419 | 1.57904 |
| Lu_{0.06}Ti_{0.98}Nb_{1.96}O₇ | 270.977 | 7.36753 | 3 | 279.1761 | | 1.54793 | 1.56889 |
| Hf_{0.06}Ti_{0.98}Nb_{1.96}O₇ | 287.8489 | 25.16989 | 4 | 274.5345 | 32.9994 | 1.55157 | 1.56926 |
| W_{0.06}Ti_{0.98}Nb_{1.96}O₇ | 286.9162 | 26.62208 | 4 | 194.2521 | | 1.55053 | 1.5779 |
| Re_{0.06}Ti_{0.98}Nb_{1.96}O₇ | 298.3182 | 22.98189 | 4 | 325.7305 | | 1.56213 | 1.56961 |
| Ir_{0.06}Ti_{0.98}Nb_{1.96}O₇ | 257.4134 | 4.08831 | 2 | 272.8497 | 31.03213 | 1.6356 | 1.63609 |
| Pt_{0.06}Ti_{0.98}Nb_{1.96}O₇ | 270.8935 | 6.18981 | 4 | 150.8053 | | 1.57335 | 1.58352 |
| Au_{0.06}Ti_{0.98}Nb_{1.96}O₇ | 286.5714 | 18.75544 | 3 | 262.894 | 25.33081 | 1.59571 | 1.61852 |
| Pb_{0.06}Ti_{0.98}Nb_{1.96}O₇ | 261.0948 | 3.22705 | 4 | 293.9583 | 41.49275 | 1.54916 | 1.58674 |
| TiNb₂O₇ | 275.8338 | 5.84256 | 50 | 274.3644 | 12.18091 | 1.58335 | 1.57063 |

TABLE 6
Capacity retention, rate return, and irreversible capacity of doped and undoped TNO materials

| | No. of | RT | 37° C. | | 37° C. | RT | 37° C. |
|---|---|---|---|---|---|---|---|
| | samples | capacity | capacity | RT rate | rate | irreversible | irreversible |
| | for RT | retention | retention | retention | retention | capacity | capacity |
| Sample | Capacity | (%) | (%) | (%) | (%) | (%) | (%) |
| B_{0.06}Ti_{0.98}Nb_{1.96}O₇ | 2 | 17.70309 | 97.60303 | 59.7217 | 81.24928 | 9.28024 | 12.21505 |
| Na_{0.06}Ti_{0.98}Nb_{1.96}O₇ | 2 | 36.87834 | 95.73795 | 74.05181 | 87.49425 | 7.31434 | 5.51652 |
| Mg_{0.06}Ti_{0.98}Nb_{1.96}O₇ | 2 | 36.32456 | 97.00083 | 70.17859 | 86.75305 | 7.13773 | 4.21232 |
| Al_{0.06}Ti_{0.98}Nb_{1.96}O₇ | 2 | 17.11415 | 95.01605 | 71.71936 | 88.5541 | 12.23768 | 7.96558 |
| Si_{0.06}Ti_{0.98}Nb_{1.96}O₇ | 2 | 24.68174 | 91.03909 | 94.35902 | 89.50235 | 11.64879 | 10.89587 |
| P_{0.06}Ti_{0.98}Nb_{1.96}O₇ | 2 | 94.95387 | 97.22433 | 83.75412 | 89.32738 | 12.43374 | 7.37658 |
| K_{0.06}Ti_{0.98}Nb_{1.96}O₇ | 2 | 33.5583 | 90.94249 | 77.89578 | 89.53282 | 36.95381 | 10.24473 |
| Ca_{0.06}Ti_{0.98}Nb_{1.96}O₇ | 2 | 48.93313 | 92.20734 | 39.00224 | 94.8325 | 10.64319 | 1.71183 |
| Sc_{0.06}Ti_{0.98}Nb_{1.96}O₇ | 1 | 5.38041 | 93.10843 | 55.81022 | 81.91027 | 15.31661 | 10.01933 |
| V_{0.06}Ti_{0.98}Nb_{1.96}O₇ | 2 | 34.92057 | 86.04154 | 73.65517 | 81.11905 | 7.90329 | 8.93474 |
| V_{0.06}Ti_{0.98}Nb_{1.96}O₇ | 2 | 56.74377 | 89.35313 | 81.69597 | 84.56458 | 4.32835 | 7.86145 |
| Cr_{0.06}Ti_{0.98}Nb_{1.96}O₇ | 2 | 70.38805 | 99.01579 | 84.00658 | 86.81471 | 8.66091 | 6.32481 |
| Mn_{0.06}Ti_{0.98}Nb_{1.96}O₇ | 2 | 75.77562 | 97.13512 | 83.71169 | 84.09572 | 3.77161 | 0.51805 |
| Fe_{0.06}Ti_{0.98}Nb_{1.96}O₇ | 2 | 71.98671 | 96.7077 | 84.08577 | 82.87939 | 6.7899 | 9.08017 |
| Co_{0.06}Ti_{0.98}Nb_{1.96}O₇ | 1 | 72.77 | 117.69 | 72.77 | 92.26502 | 12.19 | 16.179 |
| Ni_{0.06}Ti_{0.98}Nb_{1.96}O₇ | 2 | 64.61496 | 97.29765 | 77.3537 | 81.83441 | 7.26082 | 1.85651 |
| Cu_{0.06}Ti_{0.98}Nb_{1.96}O₇ | 2 | 29.82116 | 94.53685 | 62.6913 | 81.7622 | 8.89416 | 7.2727 |
| Zn_{0.06}Ti_{0.98}Nb_{1.96}O₇ | 2 | 12.70444 | 89.80719 | 32.17498 | 79.58584 | 17.80931 | 10.03495 |
| Ga_{0.06}Ti_{0.98}Nb_{1.96}O₇ | 2 | 15.35318 | 90.53375 | 53.89231 | 82.6727 | 8.84214 | 5.51083 |
| Ge_{0.06}Ti_{0.98}Nb_{1.96}O₇ | 2 | 6.60076 | 92.95825 | 15.33287 | 79.42897 | 6.52211 | 6.12971 |
| Rb_{0.06}Ti_{0.98}Nb_{1.96}O₇ | 2 | 73.09878 | 98.11166 | 85.14228 | 86.88282 | 4.48008 | 1.64863 |
| Sr_{0.06}Ti_{0.98}Nb_{1.96}O₇ | 1 | 48.7594 | 95.01293 | 78.07673 | 87.33493 | 3.67765 | 3.81729 |
| Y_{0.06}Ti_{0.98}Nb_{1.96}O₇ | 2 | 80.43551 | 96.79107 | 83.07891 | 84.56519 | 3.86085 | 10.2936 |
| Zr_{0.06}Ti_{0.98}Nb_{1.96}O₇ | 1 | 68.89623 | 95.30816 | 83.29951 | 82.55949 | 5.59223 | 2.96621 |
| Mo_{0.06}Ti_{0.98}Nb_{1.96}O₇ | 2 | 84.1192 | 97.13527 | 76.88341 | 81.26627 | 6.86406 | 5.07563 |
| Rh_{0.06}Ti_{0.98}Nb_{1.96}O₇ | 2 | 94.86679 | 100.9481 | 88.2664 | 87.32849 | 23.28609 | 19.99235 |
| Pd_{0.06}Ti_{0.98}Nb_{1.96}O₇ | 2 | 51.91301 | 84.73175 | 75.47938 | 81.78037 | 12.28402 | 6.33178 |
| Ag_{0.06}Ti_{0.98}Nb_{1.96}O₇ | 2 | 28.11994 | 70.22061 | 56.74728 | 74.47263 | 6.10374 | 7.30303 |
| Cd_{0.06}Ti_{0.98}Nb_{1.96}O₇ | 2 | 102.765 | 95.82872 | 83.31854 | 85.73066 | 3.83945 | 3.0413 |
| In_{0.06}Ti_{0.98}Nb_{1.96}O₇ | 2 | 97.30023 | 93.06076 | 78.03559 | 82.63423 | 4.04503 | 5.31476 |
| Sn_{0.06}Ti_{0.98}Nb_{1.96}O₇ | 2 | 79.70495 | 93.19881 | 84.78229 | 80.62683 | 8.34586 | 2.1358 |
| Te_{0.06}Ti_{0.98}Nb_{1.96}O₇ | 2 | 108.3078 | 97.41045 | 86.09326 | 89.65136 | 6.2301 | 6.95574 |
| Cs_{0.06}Ti_{0.98}Nb_{1.96}O₇ | 1 | 102.4258 | 94.37241 | 75.62432 | 79.43316 | 5.49015 | 2.26299 |
| Ba_{0.06}Ti_{0.98}Nb_{1.96}O₇ | 1 | 74.39001 | 93.05512 | 71.06564 | 84.23524 | 7.25022 | 6.45486 |
| La_{0.06}Ti_{0.98}Nb_{1.96}O₇ | 1 | 39.4628 | 93.23477 | 38.22951 | 73.83206 | 14.66441 | 7.76287 |
| Ce_{0.06}Ti_{0.98}Nb_{1.96}O₇ | 1 | 35.75873 | 95.43213 | 71.39219 | 86.86091 | 9.69974 | 6.35323 |
| Pr_{0.06}Ti_{0.98}Nb_{1.96}O₇ | 1 | 85.55605 | 95.82191 | 84.97367 | 91.32363 | 5.79837 | 5.12177 |
| Nd_{0.06}Ti_{0.98}Nb_{1.96}O₇ | 1 | 108.5024 | 99.2043 | 79.63129 | 82.94632 | 3.16941 | 7.76404 |
| Eu_{0.06}Ti_{0.98}Nb_{1.96}O₇ | 1 | 111.0951 | 97.41348 | 88.55562 | 87.4167 | 4.7124 | 4.92082 |
| Tb_{0.06}Ti_{0.98}Nb_{1.96}O₇ | 1 | 112.5422 | 98.45048 | 75.3675 | 79.00666 | 3.26447 | 4.0036 |
| Dy_{0.06}Ti_{0.98}Nb_{1.96}O₇ | 1 | 101.7122 | 98.19926 | 77.93545 | 77.47655 | 5.32175 | 4.94931 |
| Ho_{0.06}Ti_{0.98}Nb_{1.96}O₇ | 2 | 46.53481 | 106.8995 | 74.644 | 85.23763 | 6.75794 | 6.37614 |
| Er_{0.06}Ti_{0.98}Nb_{1.96}O₇ | 1 | 9.28613 | 85.48202 | 25.67532 | 43.83162 | 13.55897 | 10.23317 |
| Tm_{0.06}Ti_{0.98}Nb_{1.96}O₇ | 1 | 22.53094 | 93.6024 | 72.55309 | 86.72775 | 6.46831 | 5.25702 |
| Lu_{0.06}Ti_{0.98}Nb_{1.96}O₇ | 1 | 76.43334 | 97.40029 | 87.18983 | 88.7094 | 4.26798 | 3.81862 |
| Hf_{0.06}Ti_{0.98}Nb_{1.96}O₇ | 2 | 88.97341 | 97.32263 | 81.67105 | 86.6872 | 3.56247 | 4.37928 |
| W_{0.06}Ti_{0.98}Nb_{1.96}O₇ | 1 | 103.7319 | 72.06114 | 83.75302 | 68.22367 | 3.5042 | 11.21657 |
| Re_{0.06}Ti_{0.98}Nb_{1.96}O₇ | 1 | 102.0809 | 98.78181 | 83.59771 | 86.33496 | 3.93077 | 4.22496 |
| Ir_{0.06}Ti_{0.98}Nb_{1.96}O₇ | 2 | 64.27 | 96.97372 | 60.46098 | 83.94639 | 10.27 | 5.69506 |
| Pt_{0.06}Ti_{0.98}Nb_{1.96}O₇ | 1 | 54.83227 | 50.15967 | 79.01666 | 47.22757 | 4.94777 | 17.23602 |
| Au_{0.06}Ti_{0.98}Nb_{1.96}O₇ | 2 | 17.68655 | 88.19045 | 57.23354 | 85.70913 | 12.56559 | 9.23334 |
| Pb_{0.06}Ti_{0.98}Nb_{1.96}O₇ | 2 | 18.61669 | 67.37531 | 50.06074 | 60.84844 | 9.02542 | 3.52749 |
| TiNb₂O₇ | 18 | 69.91969 | 99.53085 | 81.14297 | 89.35953 | 4.72744 | 0.77818 |

TABLE 7
Electrochemical properties of additional doped TNO samples

| | RT | Error | No. | RT | RT | RT |
|---|---|---|---|---|---|---|
| | discharge | for RT | samples | rate | irreversible | average |
| | capacity | discharge | for RT | retention | capacity | voltage |
| [symbol] e | (mAh/g) | capacity | Capacity | (%) | (%) | (V) |
| Nd_{0.012}Ti_{0.9}Nb_{2.088}O₇ | 174.7505 | 12.70994 | 2 | 35.44182 | 10.78082 | 1.782484 |
| Nd_{0.012}Ti_{0.945}Nb_{2.043}O₇ | 227.3195 | 26.56092 | 2 | 51.07941 | 9.964259 | 1.607074 |
| Nd_{0.012}Ti_{0.99}Nb_{1.998}O₇ | 203.2535 | 29.5115 | 3 | 68.97834 | | 1.564342 |
| Nd_{0.012}Ti_{1.035}N_{b1.953}O₇ | 156.8077 | 36.13549 | 3 | 31.7731 | | 1.546549 |
| Nd_{0.024}Ti_{0.9}Nb_{2.076}O₇ | 236.5895 | 28.98236 | 3 | 67.19411 | | 1.567322 |
| Nd_{0.024}Ti_{0.945}Nb_{2.031}O₇ | 244.3775 | 1.267102 | 3 | 29.40323 | | 1.559093 |
| Nd_{0.024}Ti_{0.99}Nb_{1.986}O₇ | 189.9769 | 72.44125 | 3 | 12.59101 | | 1.57661 |
| Nd_{0.024}Ti_{1.035}Nb_{1.941}O₇ | 231.5813 | 18.77935 | 3 | 8.059221 | | 1.675223 |
| Nd_{0.036}Ti_{0.9}Nb_{2.064}O₇ | 287.2803 | 49.98009 | 3 | 57.76029 | | 1.571116 |
| Nd_{0.036}Ti_{0.945}Nb_{2.019}O₇ | 285.7897 | 32.62666 | 3 | 68.27786 | | 1.563518 |
| Nd_{0.036}Ti_{0.99}Nb_{1.974}O₇ | 274.2705 | 49.88241 | 3 | 73.73162 | | 1.567535 |
| Nd_{0.036}Ti_{1.035}Nb_{1.929}O₇ | 238.4765 | 35.67862 | 3 | 72.10848 | | 1.5553 |
| Nd_{0.048}Ti_{0.9}Nb_{2.052}O₇ | 232.7644 | 23.88871 | 3 | 77.92139 | | 1.562504 |
| Nd_{0.048}Ti_{0.945}Nb_{2.007}O₇ | 4.213346 | 47.29061 | 3 | 98.55902 | | 1.51621 |
| Nd_{0.048}Ti_{0.99}Nb_{1.962}O₇ | 60207.16 | 1.558866 | 3 | 55.29635 | | 2.464154 |
| Nd_{0.048}Ti_{1.035}Nb_{1.917}O₇ | 312.5095 | 1.514219 | 3 | 4.970286 | | 1.597639 |
| Nd_{0.06}Ti_{0.9}Nb_{2.04}O₇ | 252.0743 | | 1 | 57.38382 | | 1.570034 |
| Nd_{0.06}Ti_{0.945}Nb_{1.995}O₇ | 259.5546 | | 1 | 76.31272 | | 1.563492 |
| Nd_{0.06}Ti_{0.99}Nb_{1.95}O₇ | 236.3225 | | 1 | 76.26268 | | 1.556325 |
| Nd_{0.06}Ti_{1.035}Nb_{1.905}O₇ | 306.0253 | | 1 | 71.48927 | | 1.56385 |
| Nd_{0.072}T_{i0.9}Nb_{2.028}O₇ | 235.3849 | | 1 | 78.43624 | | 1.56195 |
| Nd_{0.072}Ti_{0.945}Nb_{1.983}O₇ | 255.4379 | | 1 | 75.54855 | | 1.556274 |
| Nd_{0.072}Ti_{0.99}Nb_{1.938}O₇ | 218.3527 | | 1 | 37.9504 | | 1.557975 |
| Nd_{0.072}Ti_{1.035}Nb_{1.893}O₇ | 278.671 | | 1 | 7.799667 | | 1.580577 |
| Nd_{0.084}T_{i0.9}Nb_{2.016}O₇ | 243.8067 | | 1 | 70.05481 | | 1.568999 |
| Nd_{0.084}Ti_{0.945}Nb_{1.971}O₇ | 253.6059 | | 1 | 70.48279 | | 1.565613 |
| Nd_{0.084}Ti_{0.99}Nb_{1.926}O₇ | 257.5265 | | 1 | 54.47553 | | 1.555205 |
| Nd_{0.084}Ti_{1.035}Nb_{1.881}O₇ | 261.6524 | | 1 | 64.83187 | | 1.558036 |
| Nd_{0.096}Ti_{0.9}Nb_{2.004}O₇ | 269.9523 | | 1 | 75.96098 | | 1.562103 |
| Nd_{0.096}Ti_{0.945}Nb_{1.959}O₇ | 243.664 | | 1 | 65.57157 | | 1.55528 |
| Nd_{0.096}Ti_{0.99}Nb_{1.914}O₇ | 265.7609 | | 1 | 27.74845 | | 1.558626 |
| Nd_{0.096}Ti_{1.035}Nb_{1.869}O₇ | 291.2475 | | 1 | 11.45162 | | 1.570029 |
| Tb_{0.012}Ti_{0.9}Nb_{2.088}O₇ | 265.3087 | 25.54969 | 2 | 57.58767 | 7.720739 | 1.6111 |
| Tb_{0.012}Ti_{0.945}Nb_{2.043}O₇ | 298.6444 | 47.01448 | 2 | 55.27924 | 7.694469 | 1.601886 |
| Tb_{0.012}Ti_{0.99}Nb_{1.998}O₇ | 279.1578 | 10.7588 | 3 | 67.74288 | | 1.563771 |
| Tb_{0.012}Ti_{1.035}Nb_{1.953}O₇ | 272.8427 | 38.40609 | 3 | 51.27379 | | 1.569354 |
| Tb_{0.024}Ti_{0.9}Nb_{2.076}O₇ | 240.728 | 22.49414 | 3 | 66.01466 | | 1.558437 |
| Tb_{0.024}Ti_{0.945}Nb_{2.031}O₇ | 260.5608 | 70.10553 | 2 | 52.9401 | | 1.556812 |
| Tb_{0.024}Ti_{0.99}Nb_{1.986}O₇ | 245.5302 | 42.89345 | 2 | 48.20399 | | 1.558007 |
| Tb_{0.024}Ti_{1.035}Nb_{1.941}O₇ | 300.4796 | 91.26299 | 3 | 9.249081 | | 1.566838 |
| Tb_{0.036}Ti_{0.9}Nb_{2.064}O₇ | 261.8571 | 28.87791 | 3 | 33.68507 | | 1.591149 |
| Tb_{0.036}Ti_{0.945}Nb_{2.019}O₇ | 250.4582 | 19.61411 | 3 | 81.93501 | | 1.516253 |
| Tb_{0.036}Ti_{0.99}Nb_{1.974}O₇ | 238.0265 | 27.47896 | 3 | 45.7702 | | 1.556889 |
| Tb_{0.036}Ti_{1.035}Nb_{1.929}O₇ | 217.2128 | 41.99813 | 3 | 35.52629 | | 1.554549 |
| Tb_{0.048}Ti_{0.9}Nb_{2.052}O₇ | 228.5755 | 29.29265 | 3 | 41.11377 | | 1.553921 |
| Tb_{0.048}Ti_{0.945}Nb_{2.007}O₇ | 296.8281 | 79.23709 | 2 | 57.61933 | | 1.56756 |
| Tb_{0.048}Ti_{0.99}Nb_{1.962}O₇ | 246.3945 | 49.45178 | 2 | 59.65241 | | 1.565129 |
| Tb_{0.048}Ti_{1.035}Nb_{1.917}O₇ | 207.371 | 166.4419 | 3 | 12.47 | | 1.576273 |
| Tb_{0.06}Ti_{0.9}Nb_{2.04}O₇ | 268.7622 | | 1 | 30.19111 | | 1.584561 |
| Tb_{0.06}Ti_{0.945}Nb_{1.995}O₇ | 254.2864 | | 1 | 60.74698 | | 1.555794 |
| Tb_{0.06}Ti_{0.99}Nb_{1.95}O₇ | 296.7889 | | 1 | 55.39281 | | 1.563636 |
| Tb_{0.06}Ti_{1.035}Nb_{1.905}O₇ | 256.5906 | | 1 | 26.0202 | | 1.54716 |
| Tb_{0.072}Ti_{0.9}Nb_{2.028}O₇ | 320.8305 | | 1 | 35.24966 | | 1.555753 |
| Tb_{0.072}Ti_{0.945}Nb_{1.983}O₇ | | | 0 | 121.2197 | | 2.949867 |
| Tb_{0.072}Ti_{0.99}Nb_{1.938}O₇ | 335.9819 | | 1 | 49.86194 | | 1.564924 |
| Tb_{0.072}Ti_{1.035}Nb_{1.893}O₇ | 222.6708 | | 1 | 17.46659 | | 1.572148 |
| Tb_{0.084}Ti_{0.9}Nb_{2.016}O₇ | 295.6568 | | 1 | 25.62112 | | 1.57394 |
| Tb_{0.084}Ti_{0.945}Nb_{1.971}O₇ | 280.2031 | | 1 | 35.01415 | | 1.572079 |
| Tb_{0.084}Ti_{0.99}Nb_{1.926}O₇ | 259.4391 | | 1 | 38.19599 | | 1.564912 |
| Tb_{0.084}Ti_{1.035}Nb_{1.881}O₇ | 257.1707 | | 1 | 54.31961 | | 1.563504 |
| Tb_{0.096}Ti_{0.9}Nb_{2.004}O₇ | 235.3582 | | 1 | 50.97771 | | 1.557757 |
| Tb_{0.096}Ti_{0.945}Nb_{1.959}O₇ | 270.4716 | | 1 | 50.80975 | | 1.555293 |
| Tb_{0.096}Ti_{0.99}Nb_{1.914}O₇ | 185.7469 | | 1 | 80.00487 | | 1.553434 |
| Tb_{0.096}Ti_{1.035}Nb_{1.869}O₇ | 228.3723 | | 1 | 41.6632 | | 1.568944 |
| Cs_{0.012}Ti_{0.9}Nb_{2.088}O₇ | 265.0155 | 60.38673 | 2 | 26.91384 | 7.602947 | 1.590021 |
| Cs_{0.012}Ti_{0.945}Nb_{2.043}O₇ | 127.3624 | 22.82311 | 2 | 23.80775 | 11.66715 | 1.943154 |
| Cs_{0.012}Ti_{0.99}Nb_{1.998}O₇ | 162.8708 | 34.59751 | 2 | 35.69668 | 10.31195 | 1.760139 |
| Cs_{0.012}Ti_{1.035}Nb_{1.953}O₇ | 176.0791 | 23.30765 | 2 | 32.35151 | 7.741169 | 1.873779 |
| Cs_{0.024}Ti_{0.9}Nb_{2.076}O₇ | 154.6795 | 5.635808 | 2 | 14.34825 | 7.983675 | 2.119197 |
| Cs_{0.024}Ti_{0.945}Nb_{2.031}O₇ | 188.7853 | 10.84324 | 2 | 35.21793 | 8.449654 | 1.718063 |
| Cs_{0.024}Ti_{0.99}Nb_{1.986}O₇ | 148.245 | 73.42453 | 2 | | 91.18754 | 1.769846 |
| Cs_{0.024}Ti_{1.035}Nb_{1.941}O₇ | 346.1563 | | 1 | 40.54473 | 6.785417 | 1.612273 |
| Cs_{0.036}Ti_{0.9}Nb_{2.064}O₇ | 207.837 | 36.75516 | 2 | 34.05425 | 9.139671 | 1.704687 |
| Cs_{0.036}Ti_{0.945}Nb_{2.019}O₇ | 151.8104 | 32.21106 | 2 | 32.35125 | 9.428637 | 1.85867 |
| Cs_{0.036}Ti_{0.99}Nb_{1.974}O₇ | 136.996 | 66.76039 | 2 | 89.82893 | 64.46275 | 1.677343 |
| Cs_{0.036}Ti_{1.035}Nb_{1.929}O₇ | 164.323 | 59.15571 | 2 | 43.03269 | 10.21224 | 1.598799 |
| Cs_{0.048}Ti_{0.9}Nb_{2.052}O₇ | 265.9691 | 48.82402 | 2 | 43.73519 | 6.72242 | 1.616585 |
| Cs_{0.048}Ti_{0.945}Nb_{2.007}O₇ | 273.4729 | 83.63809 | 2 | 40.69045 | 7.831694 | 1.599206 |
| Cs_{0.048}Ti_{0.99}Nb_{1.962}O₇ | 171.7258 | 28.90264 | 2 | 51.47976 | 12.15788 | 1.660681 |
| Cs_{0.048}Ti_{1.035}Nb_{1.917}O₇ | 193.9292 | 44.75567 | 2 | 53.22877 | 11.46093 | 1.615146 |
| Dy_{0.012}Ti_{0.9}Nb_{2.088}O₇ | 214.0829 | 17.15345 | 2 | 35.53014 | 7.291068 | 1.857209 |
| Dy_{0.012}Ti_{0.945}Nb_{2.043}O₇ | 247.2166 | 34.64796 | 2 | 57.58638 | 7.520762 | 1.603457 |
| Dy_{0.012}Ti_{0.99}Nb_{1.998}O₇ | 263.8865 | 31.44368 | 2 | 46.05224 | 8.163335 | 1.671042 |
| Dy_{0.012}Ti_{1.035}Nb_{1.953}O₇ | 179.4614 | 15.18234 | 2 | 47.27672 | 9.807607 | 1.675454 |
| Dy_{0.024}Ti_{0.9}Nb_{2.076}O₇ | 194.558 | 11.40579 | 2 | 3.54543 | 6.046725 | 1.864316 |
| Dy_{0.024}Ti_{0.945}Nb_{2.031}O₇ | 194.3202 | 23.87775 | 2 | 52.01932 | 6.663942 | 1.613362 |
| Dy_{0.024}Ti_{0.99}Nb_{1.986}O₇ | 130.3407 | 5.14307 | 2 | 68.8618 | 11.29238 | 1.598343 |
| Dy_{0.024}Ti_{1.035}Nb_{1.941}O₇ | 141.4098 | 33.01331 | 2 | 23.49564 | 11.01556 | 1.882146 |
| Dy_{0.036}Ti_{0.9}Nb_{2.064}O₇ | 125.171 | 47.88995 | 2 | 19.45406 | 34.93918 | 2.311759 |
| Dy_{0.036}Ti_{0.945}Nb_{2.019}O₇ | 126.4864 | 46.81657 | 2 | 24.24586 | 34.93694 | 2.25968 |
| Dy_{0.036}Ti_{0.99}Nb_{1.974}O₇ | 194.3651 | | 1 | 50.51268 | 39.82445 | 1.691724 |
| Dy_{0.036}Ti_{1.035}Nb_{1.929}O₇ | 216.5643 | 8.298924 | 2 | 36.50913 | 7.639389 | 1.772535 |
| Dy_{0.048}Ti_{0.9}Nb_{2.052}O₇ | 236.069 | 59.38538 | 2 | 14.92055 | 9.766144 | 2.076515 |
| Dy_{0.048}Ti_{0.945}Nb_{2.007}O₇ | 261.1749 | 46.72848 | 2 | 38.45009 | 9.357226 | 1.675831 |
| Dy_{0.048}Ti_{0.99}Nb_{1.962}O₇ | 206.784 | 34.63953 | 2 | 19.16283 | 5.996859 | 1.979953 |
| Dy_{0.048}Ti_{1.035}Nb_{1.917}O₇ | 160.485 | 3.501638 | 2 | 62.02953 | 12.67659 | 1.654987 |

[symbol]

indicates data missing or illegible when filed

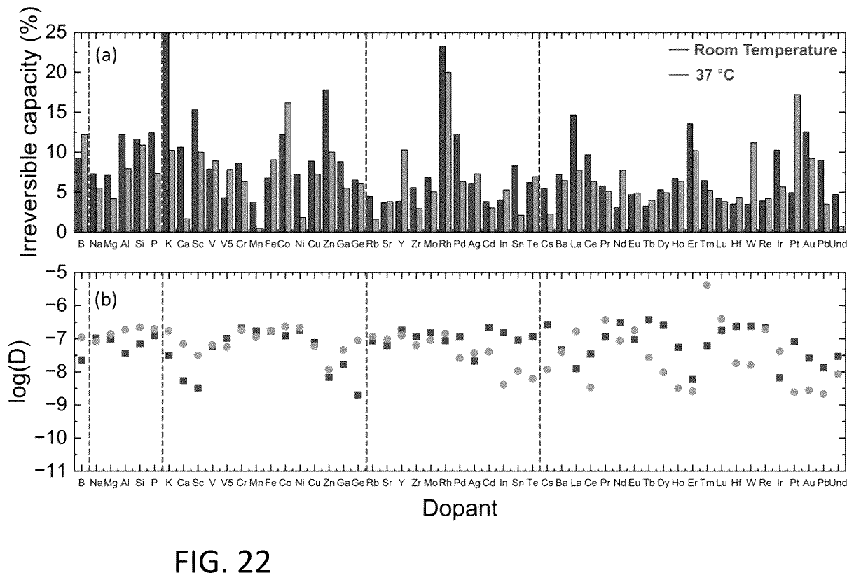

The effect of dopants on irreversible capacity was also investigated (FIG. 22 and Table 6). Most materials showed low irreversibility, under 10%, which can also be seen in the voltage curves of FIG. 25. Several strong performers such as Tb-TNO showed a very small irreversibility of 3.3%, smaller than the already small irreversibility of the undoped TNO (4.7%). Some dopants may increase the irreversibility of the cell, as seen in the K-TNO or Rh-TNO samples.

In terms of battery metrics, diffusion coefficients can be informative for a more complete understanding of material performances. Chemical diffusion coefficients were found using the Randles-Sevcik equation as described in Rehman, S.; Sieffert, J. M.; Lang, C. J.; McCalla, E. Nb_{y}W_{1-y}O_{z} and NbₓTi₁₋ₓO_{z} pseudobinaries as anodes for Li-ion batteries. Electrochimica Acta 2023, 439. DOI: 10.1016/j.electacta.2022.141665. Results are shown in FIG. 25 and Table 7.

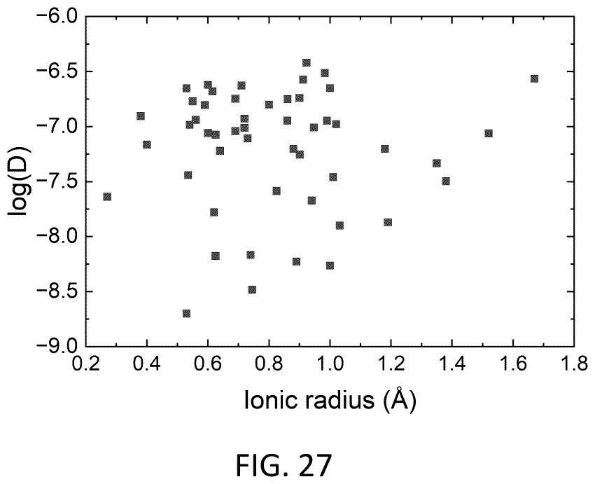

The coefficients are within the same order of magnitude of previously published Nb based anodes, indicating a high level of Li diffusivity within many of these materials (FIG. 25). A high number of dopants increase the diffusivity (D) by at least an order of magnitude over the undoped TNO, a trend especially evident at room temperature where all the high performers show improvement (e.g., Nd-TNO, Tb-TNO, and Re-TNO). This suggests that an elevated temperature and/or the appropriate dopant is required to promote Li diffusion in TNO. FIG. 27 shows the diffusion constants obtained at room temperature vs the ionic radii. The correlation is not conclusive; however, it is clear that numerous dopants are beneficial over the undoped at this lower temperature.

TABLE 8
Diffusion coefficient of doped TNO samples and undoped TNO

| | Room temperature | 37° C. |
|---|---|---|
| | Diffusion Coefficient | Diffusion Coefficient |
| Sample | (cm² s⁻¹) | (cm² s⁻¹) |
| B_{0.06}Ti_{0.98}Nb_{1.96}O₇ | 2.3E−08 | 1.09E−07 |
| Na_{0.06}Ti_{0.98}Nb_{1.96}O₇ | 1.05E−07 | 8.1E−08 |
| Mg_{0.06}Ti_{0.98}Nb_{1.96}O₇ | 9.75E−08 | 1.4E−07 |
| Al_{0.06}Ti_{0.98}Nb_{1.96}O₇ | 3.61E−08 | 1.84E−07 |
| Si_{0.06}Ti_{0.98}Nb_{1.96}O₇ | 6.85E−08 | 2.24E−07 |
| P_{0.06}Ti_{0.98}Nb_{1.96}O₇ | 1.25E−07 | 1.99E−07 |
| K_{0.06}Ti_{0.98}Nb_{1.96}O₇ | 3.18E−08 | 1.74E−07 |
| Ca_{0.06}Ti_{0.98}Nb_{1.96}O₇ | 5.44E−09 | 6.99E−08 |
| Sc_{0.06}Ti_{0.98}Nb_{1.96}O₇ | 3.28E−09 | 3.19E−08 |
| V_{0.06}Ti_{0.98}Nb_{1.96}O₇ | 6.01E−08 | 6.53E−08 |
| V_{0.06}Ti_{0.98}Nb_{1.96}O₇ | 1.04E−07 | 5.61E−08 |
| Cr_{0.06}Ti_{0.98}Nb_{1.96}O₇ | 2.09E−07 | 1.8E−07 |
| Mn_{0.06}Ti_{0.98}Nb_{1.96}O₇ | 1.71E−07 | 1.12E−07 |
| Fe_{0.06}Ti_{0.98}Nb_{1.96}O₇ | 1.69E−07 | 1.74E−07 |
| Co_{0.06}Ti_{0.98}Nb_{1.96}O₇ | 1.23E−07 | 2.36E−07 |
| Ni_{0.06}Ti_{0.98}Nb_{1.96}O₇ | 1.79E−07 | 2.19E−07 |
| Cu_{0.06}Ti_{0.98}Nb_{1.96}O₇ | 7.79E−08 | 5.9E−08 |
| Zn_{0.06}Ti_{0.98}Nb_{1.96}O₇ | 6.82E−09 | 1.2E−08 |
| Ga_{0.06}Ti_{0.98}Nb_{1.96}O₇ | 1.66E−08 | 4.63E−08 |
| Ge_{0.06}Ti_{0.98}Nb_{1.96}O₇ | 1.7E−10 | 8.95E−08 |
| Rb_{0.06}Ti_{0.98}Nb_{1.96}O₇ | 8.64E−08 | 1.17E−07 |
| Sr_{0.06}Ti_{0.98}Nb_{1.96}O₇ | 6.26E−08 | 9.75E−08 |
| Y_{0.06}Ti_{0.98}Nb_{1.96}O₇ | 1.82E−07 | 1.26E−07 |
| Zr_{0.06}Ti_{0.98}Nb_{1.96}O₇ | 1.18E−07 | 6.38E−08 |
| Mo_{0.06}Ti_{0.98}Nb_{1.96}O₇ | 1.57E−07 | 9.11E−08 |
| Rh_{0.06}Ti_{0.98}Nb_{1.96}O₇ | 8.72E−08 | 1.44E−07 |
| Pd_{0.06}Ti_{0.98}Nb_{1.96}O₇ | 1.13E−07 | 2.6E−08 |
| Ag_{0.06}Ti_{0.98}Nb_{1.96}O₇ | 2.13E−08 | 3.79E−08 |
| Cd_{0.06}Ti_{0.98}Nb_{1.96}O₇ | 2.23E−07 | 4.05E−08 |
| In_{0.06}Ti_{0.98}Nb_{1.96}O₇ | 1.58E−07 | 4.14E−09 |
| Sn_{0.06}Ti_{0.98}Nb_{1.96}O₇ | 9.07E−08 | 1.07E−08 |
| Te_{0.06}Ti_{0.98}Nb_{1.96}O₇ | 1.15E−07 | 6.17E−09 |
| Cs_{0.06}Ti_{0.98}Nb_{1.96}O₇ | 2.72E−07 | 1.18E−08 |
| Ba_{0.06}Ti_{0.98}Nb_{1.96}O₇ | 4.64E−08 | 3.94E−08 |
| La_{0.06}Ti_{0.98}Nb_{1.96}O₇ | 1.26E−08 | 1.68E−07 |
| Ce_{0.06}Ti_{0.98}Nb_{1.96}O₇ | 3.47E−08 | 3.4E−09 |
| Pr_{0.06}Ti_{0.98}Nb_{1.96}O₇ | 1.13E−07 | 3.72E−07 |
| Nd_{0.06}Ti_{0.98}Nb_{1.96}O₇ | 3.07E−07 | 8.79E−08 |
| Eu_{0.06}Ti_{0.98}Nb_{1.96}O₇ | 9.79E−08 | 1.8E−07 |
| Tb_{0.06}Ti_{0.98}Nb_{1.96}O₇ | 3.79E−07 | 2.74E−08 |
| Dy_{0.06}Ti_{0.98}Nb_{1.96}O₇ | 2.67E−07 | 9.67E−09 |
| Ho_{0.06}Ti_{0.98}Nb_{1.96}O₇ | 5.56E−08 | 3.28E−09 |
| Er_{0.06}Ti_{0.98}Nb_{1.96}O₇ | 5.92E−09 | 2.62E−09 |
| Tm_{0.06}Ti_{0.98}Nb_{1.96}O₇ | 6.26E−08 | 4.22E−06 |
| Lu_{0.06}Ti_{0.98}Nb_{1.96}O₇ | 1.78E−07 | 4E−07 |
| Hf_{0.06}Ti_{0.98}Nb_{1.96}O₇ | 2.35E−07 | 1.81E−08 |
| W_{0.06}Ti_{0.98}Nb_{1.96}O₇ | 2.39E−07 | 1.6E−08 |
| Re_{0.06}Ti_{0.98}Nb_{1.96}O₇ | 2.22E−07 | 1.87E−07 |
| Ir_{0.06}Ti_{0.98}Nb_{1.96}O₇ | 6.66E−09 | 4.16E−08 |
| Pt_{0.06}Ti_{0.98}Nb_{1.96}O₇ | 8.42E−08 | 2.44E−09 |
| Au_{0.06}Ti_{0.98}Nb_{1.96}O₇ | 2.59E−08 | 2.8E−09 |
| Pb_{0.06}Ti_{0.98}Nb_{1.96}O₇ | 1.34E−08 | 2.17E−09 |
| TiNb₂O₇ | 1.11E−07 | 6.79E−09 |

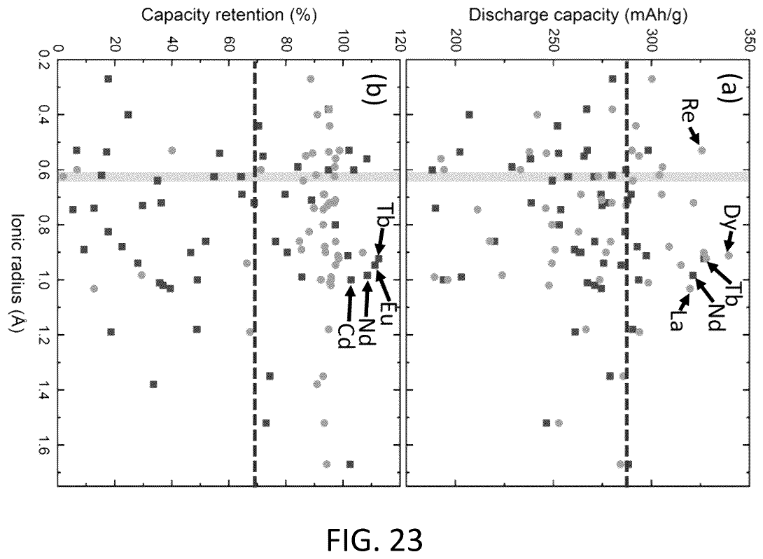

There are several approaches possible to understand the mechanisms by which the dopant atoms here might affect the performance of the TNO materials. FIG. 23 explores the relationship between the ionic radius of the substituent ions and their derived battery performance parameters. Examining the relationship between ionic radius and discharge capacity reveals that the doped materials with the largest discharge capacities contain dopants with Shannon radii between 0.9 angstroms and 1.0 angstroms, which coincides with several lanthanide dopants (FIG. 23). These radii can be compared with those of Nb and Ti ions (which have very similar radii) as seen in FIG. 23. At room temperature, substituting Nb and Ti with a dopant of a higher ionic radius may improve the discharge capacity. The presence of these dopant atoms may increase the size of the channels within the TNO crystal structure thereby allowing for easier Li diffusion leading to a larger overall discharge capacity. The 2 atom-% dopant amount in the TNO is sufficient for percolation in a next-nearest neighbor network. This suggests that the dopants disturb a volume of space around them out to the second neighbors and thereby supply strong benefits even at the low dopant levels used here. It is also worth noting that this effect is seen most strongly for several dopants in the 0.9 angstrom and 1.0 angstrom range (FIG. 27). However, there are several other dopants with a large ionic radius that do not produce materials with higher discharge capacities. Thus, it seems that the inclusion of a dopant having a larger Shannon radius in TNO may be useful to achieve high discharge capacities but may not be sufficient on its own. A similar effect can be seen for the relationship between the Shannon radius and the capacity retention of the materials. At 37° C., there is little relationship between the radii and performance, but at room temperature, several materials containing dopants having a Shannon radii the 0.9 angstrom and 1.0 angstrom range exhibit the best extended cycling performances. Again, however, it is seen that this condition alone is not sufficient for strong capacity retention. It may be possible that having subvalent ions (3+ for the lanthanides, compared to 4+, 5+ for Ti, Nb respectively) may also be beneficial. It should also be noted that Re is an outlier on the above trends; it shows improved performance despite having an ionic radius slightly smaller than Ti and Nb (though it is close enough to explain why the lattice parameter shifts were so small).

In Example 2, 52 doped-TNO samples were screened at a 2 atom-% dopant concentration. Both single and multi-phase materials were identified by PXRD. At least 47 of the dopants were integrated into the TNO structure and 24 of them were fully integrated at the tested level of 2 atom-% substitution. Several of these materials exhibited very high discharge capacities, with 19 different dopants showing significant benefit over the undoped. Specifically, several transition metal and lanthanide dopants were particularly effective, with Tb-TNO showing an initial discharge capacity of 326.7 mAh g⁻¹. At room temperature, several lanthanide-TNO samples showed high capacity retentions of over 100% after 10 cycles at 0.1 V h⁻¹, while at 37° C. the majority of materials retained their capacity above 90% over the same time frame. The inclusion of dopant atoms was shown to improve the diffusion coefficients of the materials for numerous doped samples, this in turn allows for higher discharge capacities and overall improved performance.

This application is intended to cover adaptations or variations of the present subject matter. It is to be understood that the above description is intended to be illustrative, and not restrictive, and the claims are not limited to the illustrative embodiments as set forth herein.

What is claimed is:

1. A rechargeable lithium-ion cell comprising:
a negative electrode comprising a negative electrode current collector and a negative electrode active material comprising a niobium-based oxide;
a positive electrode comprising a positive electrode current collector and a positive electrode active material comprising a lithium transition metal oxide;
a separator between the negative electrode and the positive electrode; and
an electrolyte material;
wherein the cell has an operating voltage of 1.5 V to 4 V or 1.8 V to 3.4 V.

2. The cell of claim 1, wherein the niobium-based oxide comprises Nb₂O₅, a Nb—Ti—O oxide, a Nb—W—O oxide, a Nb—Ti—W—O oxide, or a combination of two or more thereof.

3. The cell of claim 1, wherein the niobium-based oxide is represented by the formula:

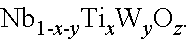

4. The cell of claim 1, wherein the niobium-based oxide is a pseudoternary composition comprising a metal portion comprising a niobium fraction, a titanium fraction, and a tungsten fraction.

5. The cell of claim 1, wherein the niobium-based oxide comprises:
a metal portion comprising:
a titanium fraction;
a niobium fraction; and
a doped fraction comprising a dopant, wherein the metal portion comprises 10 atom-% or less, 5 atom-% or less, or between 0.1 atom-% and 5 atom-% of the doped metal fraction; and
an oxygen portion.

6. The cell of claim 1, wherein the niobium-based oxide comprises:
a metal portion comprising:
a titanium fraction;
a niobium fraction; and
a doped metal fraction comprising rhenium, cerium, praseodymium, neodymium, promethium, samarium, europium, gadolinium, dysprosium, holmium, erbium, thulium, ytterbium, or a combination of two or more thereof; and
an oxygen portion.

7. The cell of claim 1, wherein the negative electrode further comprises a carbon coating, a binder, an electrically conductive additive, or a combination of two or more thereof.

8. The cell of claim 1, wherein the negative electrode active material has a lithiation potential compared to Li+/Li of between 0.5 V and 3.5 V or between 1.0 V and 3.0 V.

9. The cell of claim 1, wherein the negative electrode has a negative electrode area specific capacity of between 1 mAh/cm² and 6 mAh/cm².

10. The cell of claim 1, wherein the lithium transition metal oxide comprises LiCoO₂; LiCoMnO₄; LiCoPO₄; LiNi_{0.5}Mn_{1.5}O₄; a composition represented by the formula LiNiₓMn_{y}Co_{1-x-y}O₂; a composition represented by the formula LiNiₓCo_{y}Al_{1-x-y}O₂; a lithium-rich layered oxide represented by the formula Li₁+xTM₁−xO₂, wherein TM represents a transition metal; or a combination of two or more thereof.

11. The cell of claim 1, wherein the positive electrode active material has an operating electrode potential compared to Li+/Li of between 2.8 V and 5 V or between 3 V and 4.5 V.

12. The cell of claim 1, wherein the positive electrode has a positive electrode area specific capacity of between 1 mAh/cm² and 6 mAh/cm².

13. The cell of claim 1, wherein the negative electrode defines a negative electrode major surface and one or more negative electrode edges defining a perimeter of the negative electrode major surface;
wherein the positive electrode defines a positive electrode major surface and one or more positive electrode edges defining a perimeter of the positive electrode major surface, the one or more positive electrode edges arranged line-to-line with the one or more negative electrode edges.

14. The cell of claim 1, wherein the positive electrode has a positive electrode specific capacity and a positive electrode capacity fade rate, and the negative electrode has a negative electrode specific capacity and a negative electrode capacity fade rate;
wherein a N/P capacity ratio of the negative electrode specific capacity to the positive electrode specific capacity is less than 1; and
wherein the negative electrode capacity fade rate is less than the positive electrode capacity fade rate.

15. The cell of claim 1, wherein the positive electrode has a positive electrode specific capacity and a positive electrode capacity fade rate, and the negative electrode has a negative electrode specific capacity and a negative electrode capacity fade rate;
wherein a N/P capacity ratio of the negative electrode specific capacity to the positive electrode specific capacity is greater than 1; and
wherein the negative electrode capacity fade rate is greater than the positive electrode capacity fade rate.

16. The cell of claim 1, wherein the positive electrode has a positive electrode first-cycle irreversible capacity, and the negative electrode has a negative electrode first-cycle irreversible capacity;
wherein the negative electrode active material has an operating voltage of 3 V or greater at 95% delithiation, when the negative electrode first-cycle irreversible capacity is greater than the positive electrode first-cycle irreversible capacity; and
wherein the positive electrode active material has an operating voltage of 3 V or less at 95% lithiation, when the negative electrode first-cycle irreversible capacity is less than the positive electrode first-cycle irreversible capacity.

17. The cell of claim 1, wherein the positive electrode has a positive electrode first-cycle irreversible capacity, and the negative electrode has a negative electrode first-cycle irreversible capacity;
wherein the negative electrode active material has an operating voltage of 3 V or greater at 95% delithiation; and
wherein the negative electrode first-cycle irreversible capacity is greater than the positive electrode first-cycle irreversible capacity.

18. The cell of claim 1, wherein the positive electrode has a positive electrode first-cycle irreversible capacity, and the negative electrode has a negative electrode first-cycle irreversible capacity;
wherein the positive electrode active material has an operating voltage of 3 V or less at 95% lithiation; and
wherein the negative electrode first-cycle irreversible capacity is less than the positive electrode first-cycle irreversible capacity.

19. The cell of claim 1, wherein the positive electrode has a positive electrode first-cycle irreversible capacity, and the negative electrode has a negative electrode first-cycle irreversible capacity;
wherein the negative electrode active material has an operating voltage of 3 V or greater at the end of near full delithiation;
wherein the negative electrode first-cycle irreversible capacity is greater than the positive electrode first-cycle irreversible capacity; and
wherein the negative electrode current collector comprises aluminum.

20. The cell of claim 1, wherein the positive electrode has a positive electrode first-cycle irreversible capacity, and the negative electrode has a negative electrode first-cycle irreversible capacity;
wherein the negative electrode active material has an operating voltage of 3 V or less at the end of near full delithiation;
wherein the negative electrode first-cycle irreversible capacity is less than the positive electrode first-cycle irreversible capacity; and
wherein the negative electrode current collector comprises copper.
